SEQUENCE LISTING (37 sequences)

SEQ ID NO: 1 - DNA, 3052 bp, Bos taurus
gaattcaacaattctatttatcaagaaatctcccacaaatatactcacactgtgctccaataagtacttttgagtctattaattaacaacattatgtatctaacattatggttagattgattatggtgcatctgtgaaatgaaacatacgctgactctaaaatgattggcacaatcctgtgtattggtaagaaattgtattctagatatactaggtgaaagaagtgagattaataatggttaaaattgggcaatggtacatggacatttgtaatgccatttttccttataagtgtattgtggagatttaaatttttccaaaaaaaaaaatgtggaggcagggggcaagaacattagtgtgaataatatgacattatttaaatgcccttaaatatatattttttaattaatttatttattttttctatgctgggtcttcattgcaatgtgcaagcttctcactgtggcagcttctcctgttgtggagcataggctccaggtgcccagggactcagcggttgcaacacacaggctctagagcttgggctcgggagccgtgtcacacggcctttgttgctcccagcatgtggaatcttcctcaaccagggaccgatcccgcgtccctgcactggcaggcggattctcatccactgtaccaccaaggaaggcctgcacacttttttttttaaggaaatggatatatgaaggacagaaaaagaatatccatggaaggatacaccataaactgagaagaacaactacttctagggaaaaaaggactggggagagactgagttttcatatctttgttccttttgaattttaagaaaaataatacattattctaacacaacattgtaaagcaattatacttcaataaaaaattaaaagtaaaaatactttattatataataatatataattataatataatataaacattcagttcagttcagttcacttcagtcgctcagtcgtgtccgactcttttcgaccccatgaatcgcagcacaccaggcctccctgtccatcaccaactcccggagttcacccagactcatagtaatatataataatttattttaaaataattattaatcaacacgaaatgtaaaaaataggtaggtgatgggtagataggcagacgggcagtccacacactcacatgtggtctcaagtgctacttggtgttcaggcaataactctggtcccaatctgacctctgacccttaaaaaggtgatggtaagacaagtaacctgaggctgccagggcccctgcctatgagctaagactctgcttagaaccaagttacaaagatgttgcagacaagaaaaatttggtcgtagtggatgctactgcctctatttgaaaaacaacacaaacatttccggggggggggggaggcggagaggaggaaagattttcttcaaaatgtaatttcattgtagacacttctttaaaagaaacatttctttatttgacagttccaggccttagtttcagcaggcaggatgtttagtcgcagcatgagaactcttagctgcggcatgcgggacccagttcagttccctgaccagatatcgaacctggggcccctgcatttggaagcagggagtcttagccactggaccaccagggaagtcccctgtagatgtttttatgaaaagcagaaaagcacaaagaagagcttaaagattcctgatcctactcccaatagtgataatgtatattttggtgtgagagtgtgtgtattgattggaatgtgtgtgatcagaaaacacataccattttataatccgttctttccagctcacaaaataaagttattttcctacatcattaaatattactttacaacataatttttaatgtgtgcatattgctgctatgtgattttcaataacttactaatttcctatgctgaacatttagttgttgtccaacctttttagtggccatgtaattataaatcatggtcaatgctaacaatttctgacctcacaaacatatagtacaatatccttcctttcttcaatagataattattaaaagcaaaacaaccaggctcaaacaaagcaattataaaatatctttaaaaagacattgggtaaaattcaaatgcagactagctcatgatgttaaagaattactcttgtgtggtaatggtcttgtgatagagatagaaatgcttccttatttttcagataaacacttaagtatttaaggatgaaacgccctgatgtttgtaatttgctttagaatattttagccaaaagaattaatgatgcaaatatgcaaaaagagtacgttaaacctaaatttgcgattttcatttaaaaatatatcttaaaaatgaaaatcttcgtgcaacgcacggggctatcaatgtgggatacagatgtgaacaaaacggacccgtgtgggactcggcggagcacacagattttgcgggagcacgttcccgttaggaagtctctgatgcaatacgaccggtgcccttcaggacctgtgagactgactttccttacccctccacaccatcatcaaggcaggtgtgattttccaggccaggcctacggccgggtttccccgggggcccagagccgtcgggtcttgccgcccagcggagctggctgctccggcctcactgtcggggcgccaccgcccccagccggctcagaggaacccctcaccgccaccctgtcccaggcggcctttccccgaggcccgagggtcagatcctggggccacctcgaggatttctcacacctgcccagccacccccagcttttcaggtgataccggagggtgggcgtggggctcctggcgcatccgagtccctccctggagtccccgaccgcggccgcccggcccgacgctgccccgccgccccgcagggcgggagccggcgctgcgggtgcgccccggccagccgggcagttgcgcaagttgtgcttcggcggctataagaggggcgggcaggcatggagccccggagggatcgaggaatcgcggcgccagcagcggcgaggtaagtgcccggctctctcct

SEQ ID NO: 2 - DNA, 3052 bp, Bos taurus
gaattcaacaattctatttatcaagaaatctcccacaaatatactcacactgtgctccaataagtacttttgagtctattaattaacaacattatgtatctaacattatggttagattgattatggtgcatctgtgaaatgaaacatacgctgactctaaaatgattggcacaatcctgtgtattggtaagaaattgtattctagacatactaggtgaaagaagtgagattaataatggttaaaattgggcaatggtacatggacatttgtaatgccatttttccttataagtgtattgtggagatttaaatttttccaaaaaaaaaaatgtggaggcagggggcaagaacattagtgtgaataatatgacattatttaaatgcccttaaatatatattttttaattaatttatttattttttctatgctgggtcttcattgcaatgtgcaagcttctcactgtggcagcttctcctgttgtggagcataggctccaggtgcccagggactcagcggttgcaacacacaggctctagagcttgggctcgggagccgtgtcacacggcctttgttgctcccagcatgtggaatcttcctcaaccagggaccgatcccgcgtccctgcactggcaggcggattctcatccactgtaccaccaaggaaggcctgcacacttttttttttaaggaaatggatatatgaaggacagaaaaagaatatccatggaaggatacaccataaactgagaagaacaactacttctagggaaaaaaggactggggagagactgagttttcatatctttgttccttttgaattttaagaaaaataatacattattctaacacaacattgtaaagcaattatacttcaataaaaaattaaaagtaaaaatactttattatataataatatataattataatataatataaacattcagttcagttcagttcacttcagtcgctcagtcgtgtccgactcttttcgaccccatgaatcgcagcacaccaggcctccctgtccatcaccaactcccggagttcacccagactcatagtaatatataataatttattttaaaataattattaatcaacacgaaatgtaaaaaataggtaggtgatgggtagataggcagacgggcagtccacacactcacatgtggtctcaagtgctacttggtgttcaggcaataactctggtcccaatctgacctctgacccttaaaaaggtgatggtaagacaagtaacctgaggctgccagggcccctgcctatgagctaagactctgcttagaaccaagttacaaagatgttgcagacaagaaaaatttggtcgtagtggatgctactgcctctatttgaaaaacaacacaaacatttccggggggggggggaggcggagaggaggaaagattttcttcaaaatgtaatttcattgtagacacttctttaaaagaaacatttctttatttgacagttccaggccttagtttcagcaggcaggatgtttagtcgcagcatgagaactcttagctgcggcatgcgggacccagttcagttccctgaccagatatcgaacctggggcccctgcatttggaagcagggagtcttagccactggaccaccagggaagtcccctgtagatgtttttatgaaaagcagaaaagcacaaagaagagcttaaagattcctgatcctactcccaatagtgataatgtatattttggtgtgagagtgtgtgtattgattggaatgtgtgtgatcagaaaacacataccattttataatccgttctttccagctcacaaaataaagttattttcctacatcattaaatattactttacaacataatttttaatgtgtgcatattgctgctatgtgattttcaataacttactaatttcctatgctgaacatttagttgttgtccaacctttttagtggccatgtaattataaatcatggtcaatgctaacaatttctgacctcacaaacatatagtacaatatccttcctttcttcaatagataattattaaaagcaaaacaaccaggctcaaacaaagcaattataaaatatctttaaaaagacattgggtaaaattcaaatgcagactagctcatgatgttaaagaattactcttgtgtggtaatggtcttgtgatagagatagaaatgcttccttatttttcagataaacacttaagtatttaaggatgaaacgccctgatgtttgtaatttgctttagaatattttagccaaaagaattaatgatgcaaatatgcaaaaagagtacgttaaacctaaatttgcgattttcatttaaaaatatatcttaaaaatgaaaatcttcgtgcaacgcacggggctatcaatgtgggatacagatgtgaacaaaacggacccgtgtgggactcggcggagcacacagattttgcgggagcacgttcccgttaggaagtctctgatgcaatacgaccggtgcccttcaggacctgtgagactgactttccttacccctccacaccatcatcaaggcaggtgtgattttccaggccaggcctacggccgggtttccccgggggcccagagccgtcgggtcttgccgcccagcggagctggctgctccggcctcactgtcggggcgccaccgcccccagccggctcagaggaacccctcaccgccaccctgtcccaggcggcctttccccgaggcccgagggtcagatcctggggccacctcgaggatttctcacacctgcccagccacccccagcttttcaggtgataccggagggtgggcgtggggctcctggcgcatccgagtccctccctggagtccccgaccgcggccgcccggcccgacgctgccccgccgccccgcagggcgggagccggcgctgcgggtgcgccccggccagccgggcagttgcgcaagttgtgcttcggcggctataagaggggcgggcaggcatggagccccggagggatcgaggaatcgcggcgccagcagcggcgaggtaagtgcccggctctctcct

SEQ ID NO: 3 - DNA, 3052 bp, Bos taurus
gaattcaacaattctatttatcaagaaatctcccacaaatatactcacactgtgctccaataagtacttttgagtctattaattaacaacattatgtatctaacattatggttagattgattatggtgcatctgtgaaatgaaacatacgctgactctaaaatgattggcacaatcctgtgtattggtaagaaattgtattctagatatactaggtgaaagaagtgagattaataatggttaaaattgggcaatggtacatggacatttgtaatgccatttttccttataagtgtattgtggagatttaaatttttccaaaaaaaaaaatgtggaggcagggggcaagaacattagtgtgaataatatgacattatttaaatgcccttaaatatatattttttaattaatttatttattttttctatgctgggtcttcattgcaatgtgcaagcttctcactgtggcagcttctcctgttgtggagcataggctccaggtgcccagggactcagcggttgcaacacataggctctagagcttgggctcgggagccgtgtcacacggcctttgttgctcccagcatgtggaatcttcctcaaccagggaccgatcccgcgtccctgcactggcaggcggattctcatccactgtaccaccaaggaaggcctgcacacttttttttttaaggaaatggatatatgaaggacagaaaaagaatatccatggaaggatacaccataaactgagaagaacaactacttctagggaaaaaaggactggggagagactgagttttcatatctttgttccttttgaattttaagaaaaataatacattattctaacacaacattgtaaagcaattatacttcaataaaaaattaaaagtaaaaatactttattatataataatatataattataatataatataaacattcagttcagttcagttcacttcagtcgctcagtcgtgtccgactcttttcgaccccatgaatcgcagcacaccaggcctccctgtccatcaccaactcccggagttcacccagactcatagtaatatataataatttattttaaaataattattaatcaacacgaaatgtaaaaaataggtaggtgatgggtagataggcagacgggcagtccacacactcacatgtggtctcaagtgctacttggtgttcaggcaataactctggtcccaatctgacctctgacccttaaaaaggtgatggtaagacaagtaacctgaggctgccagggcccctgcctatgagctaagactctgcttagaaccaagttacaaagatgttgcagacaagaaaaatttggtcgtagtggatgctactgcctctatttgaaaaacaacacaaacatttccggggggggggggaggcggagaggaggaaagattttcttcaaaatgtaatttcattgtagacacttctttaaaagaaacatttctttatttgacagttccaggccttagtttcagcaggcaggatgtttagtcgcagcatgagaactcttagctgcggcatgcgggacccagttcagttccctgaccagatatcgaacctggggcccctgcatttggaagcagggagtcttagccactggaccaccagggaagtcccctgtagatgtttttatgaaaagcagaaaagcacaaagaagagcttaaagattcctgatcctactcccaatagtgataatgtatattttggtgtgagagtgtgtgtattgattggaatgtgtgtgatcagaaaacacataccattttataatccgttctttccagctcacaaaataaagttattttcctacatcattaaatattactttacaacataatttttaatgtgtgcatattgctgctatgtgattttcaataacttactaatttcctatgctgaacatttagttgttgtccaacctttttagtggccatgtaattataaatcatggtcaatgctaacaatttctgacctcacaaacatatagtacaatatccttcctttcttcaatagataattattaaaagcaaaacaaccaggctcaaacaaagcaattataaaatatctttaaaaagacattgggtaaaattcaaatgcagactagctcatgatgttaaagaattactcttgtgtggtaatggtcttgtgatagagatagaaatgcttccttatttttcagataaacacttaagtatttaaggatgaaacgccctgatgtttgtaatttgctttagaatattttagccaaaagaattaatgatgcaaatatgcaaaaagagtacgttaaacctaaatttgcgattttcatttaaaaatatatcttaaaaatgaaaatcttcgtgcaacgcacggggctatcaatgtgggatacagatgtgaacaaaacggacccgtgtgggactcggcggagcacacagattttgcgggagcacgttcccgttaggaagtctctgatgcaatacgaccggtgcccttcaggacctgtgagactgactttccttacccctccacaccatcatcaaggcaggtgtgattttccaggccaggcctacggccgggtttccccgggggcccagagccgtcgggtcttgccgcccagcggagctggctgctccggcctcactgtcggggcgccaccgcccccagccggctcagaggaacccctcaccgccaccctgtcccaggcggcctttccccgaggcccgagggtcagatcctggggccacctcgaggatttctcacacctgcccagccacccccagcttttcaggtgataccggagggtgggcgtggggctcctggcgcatccgagtccctccctggagtccccgaccgcggccgcccggcccgacgctgccccgccgccccgcagggcgggagccggcgctgcgggtgcgccccggccagccgggcagttgcgcaagttgtgcttcggcggctataagaggggcgggcaggcatggagccccggagggatcgaggaatcgcggcgccagcagcggcgaggtaagtgcccggctctctcct

SEQ ID NO: 4 - DNA, 277 bp, Bos taurus
tgttggacgagatcatttcccaaccaaagctctgagaacctgtgacaattatgccttgagatattgatcgatgtgcagtaccgccattttccatgtggacaatggcatgaaaaaagattaggataaccttatctaaagctatctaaggacrcttctagctctcaggttccatttatttcaaagagccacttcctgaaacagctttttctctgttggataggtttgtaagaaaattcttactagtgtcatattgagctgagagtctttcactggcccg

SEQ ID NO: 5 - DNA, 467 bp, Bos taurus
gattccgccgcacctctccccaggggagtgcctttcattactgtcatttctagacaatgaattgtctttgaggagatgatagccatggcagacagcaaatcttgttgttatccgcatctgaagacctggatgcgggtggtaacggagcacgtgggtgttctcggagatcgacgatgtgccacgtgtggtttcttctgttttcaggccccagaagcccatcccgggaaggaaaatgcgctgtggacccctgtttcgattcctgtggctttggccctatctgtcttacgtggaggctgtgcccatctgcaaggtccaggatgacaccaaaaccctcatcaagacaattgtcaccaggatcaatgacatctcacacacggtagggagggactgggagacgaggtagaaccgtggccatcccgtgggggaccccagaggctggcggaggaggctgtgcagccttgcacagg

SEQ ID NO: 6 - DNA, 467 bp, Bos taurus
gattccgccgcacctctccccaggggagtgcctttcattactgtcatttctagacaatgaattgtctttgaggagatgatagccatggcagacagcaaatcttgttgttatccgcatctgaagacctggatgcgggtggtaacggagcacgtgggtgttctcggagatcgacgatgtgccacgtgtggtttcttctgttttcaggccccagaagcccatcccgggaaggaaaatgcgctgtggacccctgtttcgattcctgtggctttggccctatctgtcttacgtggaggctgtgcccatccgcaaggtccaggatgacaccaaaaccctcatcaagacaattgtcaccaggatcaatgacatctcacacacggtagggagggactgggagacgaggtagaaccgtggccatcccgtgggggaccccagaggctggcggaggaggctgtgcagccttgcacagg

SEQ ID NO: 7 - DNA, 25 bp, Artificial Sequence
  Description of Artificial Sequence Synthetic primer
ggcacaatcctgtgtattggtaaga

SEQ ID NO: 8 - DNA, 23 bp, Artificial Sequence
  Description of Artificial Sequence Synthetic primer
gtccatgtaccattgcccaattt

SEQ ID NO: 9 - DNA, 17 bp, Artificial Sequence
  Description of Artificial Sequence Synthetic primer
aggtgcccagggactca

SEQ ID NO: 10 - DNA, 19 bp, Artificial Sequence
  Description of Artificial Sequence Synthetic primer
caacaaaggccgtgtgaca

SEQ ID NO: 11 - DNA, 23 bp, Artificial Sequence
  Description of Artificial Sequence Synthetic primer
ggctttggccctatctgtcttac

SEQ ID NO: 12 - DNA, 22 bp, Artificial Sequence
  Description of Artificial Sequence Synthetic primer
cttgatgagggttttggtgtca

SEQ ID NO: 13 - DNA, 20 bp, Artificial Sequence
  Description of Artificial Sequence Synthetic probe
ctttcacctagtatatctag

SEQ ID NO: 14 - DNA, 21 bp, Artificial Sequence
  Description of Artificial Sequence Synthetic probe
tctttcacctagtatgtctag

SEQ ID NO: 15 - DNA, 18 bp, Artificial Sequence
  Description of Artificial Sequence Synthetic probe
aagctctagagcctatgt

SEQ ID NO: 16 - DNA, 19 bp, Artificial Sequence
  Description of Artificial Sequence Synthetic probe
caagctctagagcctgtgt

SEQ ID NO: 17 - DNA, 13 bp, Artificial Sequence
  Description of Artificial Sequence Synthetic probe
ccttgcagatggg

SEQ ID NO: 18 - DNA, 13 bp, Artificial Sequence
  Description of Artificial Sequence Synthetic probe
ccttgcggatggg

SEQ ID NO: 19 - DNA, 197 bp, Bos taurus
ttcattgtagacacttctttaaaagaaacatttctttatttgacagttccaggccttagtttcagcaggcaggatgtttagtcgcagcatgagaactcttarctgcggcatgcgggacccagttcagttccctgaccagatatcgaacctggggcccctgcatttggaagcagggagtcttagccactggaccacca

SEQ ID NO: 20 - DNA, 301 bp, Bos taurus
cccttccattttagggctcgttgacgtcatcaaggaggcgataaatatctgttgatataattggatgtgagattcagtgttgagatagcaaaaattctgcccctcgttcccgggcagggccctatgatttatgcaggagcagaggcagcgygcaatccagctgtcaagagagcgtcagcttattaggcaaatgctgcgtggtttctgaagagggtcgacactataaaatccccttccaggctctggtgtggagaaactcagagcccacgtccgtggagagacagaagaggaagagaagagg

SEQ ID NO: 21 - DNA, 291 bp, Bos taurus
aagaaaattcttactagtgtcatattgagctgagagtctttcactggcccgttgagtgcgaccaccaagcccacttctaagtggtaagcaactatcaccacttccttttcaaaaattctcagtattgaaagggaccttttagatcatctgkccaaatctctcactctgtcatggaagaaacagggttggattttgtttgtttgattattttctgagtacacattatgtacaaggccacaaacttagataaaaaacacagccttcttttcaccgcccatgaagtctagtggc

SEQ ID NO: 22 - DNA, 30 bp, Artificial Sequence
  Description of Artificial Sequence Synthetic primer
acgttggatgtgcctaataagctgacgctc

SEQ ID NO: 23 - DNA, 30 bp, Artificial Sequence
  Description of Artificial Sequence Synthetic primer
acgttggatggcagggccctatgatttatg

SEQ ID NO: 24 - DNA, 30 bp, Artificial Sequence
  Description of Artificial Sequence Synthetic primer
acgttggatgaaaattctgcccctcgttcc

SEQ ID NO: 25 - DNA, 30 bp, Artificial Sequence; identical to SEQ ID NO: 22
  Description of Artificial Sequence Synthetic primer

SEQ ID NO: 26 - DNA, 30 bp, Artificial Sequence
  Description of Artificial Sequence Synthetic primer
acgttggatgtcagcaggcaggatgtttag

SEQ ID NO: 27 - DNA, 30 bp, Artificial Sequence
  Description of Artificial Sequence Synthetic primer
acgttggatgatctggtcagggaactgaac

SEQ ID NO: 28 - DNA, 30 bp, Artificial Sequence; identical to SEQ ID NO: 26
  Description of Artificial Sequence Synthetic primer

SEQ ID NO: 29 - DNA, 30 bp, Artificial Sequence; identical to SEQ ID NO: 27
  Description of Artificial Sequence Synthetic primer

SEQ ID NO: 30 - DNA, 28 bp, Artificial Sequence
  Description of Artificial Sequence Synthetic primer
acgttggatgtccatgacagagtgagag

SEQ ID NO: 31 - DNA, 28 bp, Artificial Sequence
  Description of Artificial Sequence Synthetic primer
acgttggatgttgaaagggaccttttag

SEQ ID NO: 32 - DNA, 30 bp, Artificial Sequence
  Description of Artificial Sequence Synthetic primer
acgttggatgcttccatgacagagtgagag

SEQ ID NO: 33 - DNA, 30 bp, Artificial Sequence
  Description of Artificial Sequence Synthetic primer
acgttggatgaagcaactatcaccacttcc

SEQ ID NO: 34 - DNA, 30 bp, Artificial Sequence
  Description of Artificial Sequence Synthetic primer
acgttggatgaataaatggaacctgagagc

SEQ ID NO: 35 - DNA, 30 bp, Artificial Sequence
  Description of Artificial Sequence Synthetic primer
acgttggatggtaccgccattttccatgtg

SEQ ID NO: 36 - DNA, 30 bp, Artificial Sequence
  Description of Artificial Sequence Synthetic primer
acgttggatgttttccatgtggacaatggc

SEQ ID NO: 37 - DNA, 30 bp, Artificial Sequence; identical to SEQ ID NO: 34
  Description of Artificial Sequence Synthetic primer

(19) United States
(12) Patent Application Publication
(10) Pub. No.: US 2008/0160523 A1
(43) Pub. Date: Jul. 3, 2008

(54) Association of Single Nucleotide Polymorphisms, Dairy Form and Productive Life

(76) Inventors: Brent Woodward, Lawrenceville, GA (US); Donald Joshua Nkrumah, Edmonton (CA)

Correspondence Address: FROMMER LAWRENCE & HAUG, 745 FIFTH AVENUE- 10TH FL., NEW YORK, NY, 10151, US

(21) Appl. No.: 11/837,193

(22) Filed: Aug. 10, 2007

Related U.S. Application Data
(60) Provisional application No. 60/836,991, filed on Aug. 10, 2006.

(51) Int. Cl. C12Q 1/68; G06F 17/30

(52) U.S. Cl. 435/6; 707/104.1; 707/E17.009

(57) ABSTRACT

The physiological regulation of intake, growth and energy partitioning in animals is under the control of multiple genes, which may be important candidates for unraveling the genetic variation in economically relevant traits in beef production. The present invention relates to the identification of a single nucleotide polymorphisms (SNPs) within the bovine genes encoding leptin, corticotropin-releasing hormone, and mitochondrial transcription factor A and their association with the economically relevant traits of dairy form and productive life. The invention further encompasses methods and systems, including network-based processes, to manage the SNP data and other data relating to specific animals and herds of animals, veterinarian care, diagnostic and quality control data and management of livestock which, based on genotyping, have predictable meat quality traits, husbandry conditions, animal welfare, food safety information, audit of existing processes and data from field locations.

### INCORPORATION BY REFERENCE

This application claims priority to U.S. Provisional Patent Application No. 60/836,991 filed Aug. 10, 2006. Reference is made to U.S. application Ser. No. 11/061,942 filed Feb. 19, 2005.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

More generally, documents or references are cited in this text, either in a Reference List before the claims, or in the text itself, and, each of these documents or references (“herein cited references”), as well as each document or reference cited in each of the herein-cited references (including any manufacturer's specifications, instructions, etc.), is hereby expressly incorporated herein by reference.

### FIELD OF THE INVENTION

The present invention relates to single nucleotide polymorphisms in the leptin or ob gene, and to the association of these SNPs with certain traits that are economically important in livestock species, including dairy form (DF) and productive life (PL). The present invention provides methods of identifying and grouping animals based on their genotype.

### BACKGROUND OF THE INVENTION

Significant improvements in animal performance, efficiency and carcass and meat quality have been made over the years through the application of standard animal breeding and selection techniques. However, such classical animal breeding techniques require several years of genetic evaluation of performance records on individual animals and their relatives and are therefore very expensive. Other efforts have been made to improve productivity and quality through the application of such management practices as the use of feed additives, animal hormonal implants and chemotherapeutics. However, there is significant political and regulatory resistance to the introduction and use of such methodologies. Such methodologies are also non-inheritable and need to be applied differently in every production system.

There is a need for methods that allow relatively easy and more efficient selection and breeding of farm animals with an advantage for an inheritable trait of circulating leptin levels, feed intake, growth rate, body weight, carcass merit and carcass composition. The economic significance of the use of genetic markers that are associated with specific economically important traits (especially traits with low heritability) in livestock through marker-assisted selection cannot therefore be over-emphasized.

The physiological regulation of intake, growth and energy partitioning in animals is under the control of multiple genes, which may be important candidates for unraveling the genetic variation in economically relevant traits (ERT) in beef production. Polymorphisms in these candidate genes that show association with specific ERT are useful quantitative trait nucleotides for marker-assisted selection. For example, SNPs (SNPs) in the bovine growth hormone receptor (GHR), bovine neuropeptide Y (NPY), leptin, ghrelin and uncoupling protein 2 (UCP2) genes are known to be associated with measures of intake, growth and carcass merit in beef cattle.

Leptin has been proposed as one of the major control factors contributing to the phenotypic and genetic variation in the performance and efficiency of cattle. Leptin is a 16-kDa adipocyte-specific polypeptide, expressed predominantly in fat tissues of those animals in which it has been detected including livestock species such as cattle, pigs, and sheep. Leptin is encoded by the ob (obese) gene and appears to be involved in the regulation of appetite, basal metabolism and fat deposition. Increased plasma concentrations of leptin in mice, cattle, pigs and sheep have been associated with decreased body fat deposition and appetite, and increased basal metabolism levels (Blache et al., Reprod Fertil Dev. 2000; 12(7-8):373-81; Blache et al., J Reprod Fertil. 2000 September; 120(1): 1-11; Blache et al., J Endocrinol. 2000 June; 165(3):625-37; Delavaud et al., J Endocrinol. 2000 May; 165(2):519-26; Ehrhardt et al., J Endocrinol. 2000 September; 166(3):519-28). Similar phenotypic characteristics have also been found to be associated with leptin mRNA levels in adipose tissue (Ramsay et al., J Anim Sci. 1998 February; 76(2):484-90; Roberts et al., Clin Endocrinol (Oxf). 1998 April; 48(4):401-6). Consistent with those observations, it has been shown that administration of exogenous leptin dramatically reduces feed intake and body mass of mice, chickens, pigs and sheep (Barb et al., Domest Anim Endocrinol. 1998 January; 15(1):77-86; Halaas et al., Science. 1995 Jul. 28; 269(5223):543-6; Henry et al., Endocrinology. 1999 March; 140(3):1175-82; and Raver et al., Protein Expr Purif. 1998 December; 14(3):403-8).

The ob gene has been mapped to chromosome 6 in mice (Friedman et al., Genomics. 1991 December; 11(4):1054-62; Friedman et al., Mamm Genome. 1991; 1(3):130-44), chromosome 7q31.3 in humans (Isse et al., J Biol Chem. 1995 Nov. 17; 270(46):27728-33) chromosome 4 in cattle (Stone et al., Mamm Genome. 1996 May; 7(5):399-400), and chromosome 18 in swine (Neuenschwander et al., Anim Genet. 1996 August; 27(4):275-8; Sasaki et al., Mamm Genome. 1996 June; 7(6):471-2). Sequences have been determined for the said gene from mice (Zhang et al., Nature. 1994 Dec. 1; 372(6505):425-32), cattle (U.S. Pat. No. 6,297,027 to Spurlock), pigs (U.S. Pat. No. 6,277,592 to Bidwell and Spurlock; Neuenschwander et al., 1996), and humans (U.S. Pat. No. 6,309,857 to Friedman et al.) and there is significant conservation among the sequences of ob DNAs and leptin polypeptides from those species (Ramsay et al. J Anim Sci. 1998 February; 76(2):484-90).

Studies in humans have shown that mutations in the CCAAT/enhancer binding protein (C/EBP-alpha) region of the leptin promoter abolished inducibility of the promoter by C/EBP-.alpha. (Miller et al., Proc Natl Acad Sci USA. 1996 May 28; 93(11):5507-11). Mason et al. (Endocrinology. 1998 March; 139(3):1013-22) have shown that mutations in the C/EBP-.alpha. and TATA motifs as well as in a consensus Sp1 site of leptin reduced promoter activity by 10, 10 and 2.5-fold, respectively, and abolished binding of these factors. Mason et al. (1998) also showed that the regulation of leptin gene expression is partly linked to a novel factor that binds to an LP1 motif in the promoter. The role of peroxizome proliferator activated receptor-.gamma. (PPAR-gamma) in adipocyte differentiation has also been linked to leptin promoter function (De Vos et al., J Clin Invest. 1996 Aug. 15; 98(4):1004-9).

It has been demonstrated that plasma leptin concentrations are significantly diminished in animals homozygous for mutant alleles of the ob gene (ob.−/ob.− animals), which alleles do not encode functional leptin, compared to wild-type (ob.+/ob.+) controls. Mutations in the coding sequences of the ob gene causing alterations in the amino acid sequence of the leptin polypeptide, have been associated with hyperphagia, hypometabolic activity, and excessive fat deposition; i.e., a phenotype characterized by larger body size; a fat phenotype (Zhang et al., Nature. 1994 Dec. 1; 372(6505):425-32).

Fitzsimmons et al. (Mamm Genome. 1998 June; 9(6):432-4) reported evidence of three alleles of a microsatellite marker located proximal to the ob gene in cattle that occurred with significant frequency in bulls of several breeds (Angus, Charolais, Hereford and Simmental) and comprising 138, 147 and 149 base pairs (bp). The 138-bp and 147-bp alleles, respectively, occurred most frequently. Further, it was determined that occurrence of the 138-bp allele was positively associated with certain carcass characteristics; increased average fat deposition, increased mean fat deposition, increased percent rib fat, and decreased percent rib lean. Thus, bulls homozygous for the 138-bp allele exhibited greater average fat deposition than heterozygous animals and such heterozygotes exhibited greater average fat deposition that bulls homozygous for the 147-bp allele.

Several SNPs have been reported in the leptin gene (Buchanan et al., Genet. Sel. Evol. (2002) 34:105-116; Lagonigro et al., Anim. Genet. (2003) 34:371-374; Nkrumah, J. D. (2004) Can. J. Anim. Sci. (2004) 84:211-219). Associations of molecular polymorphisms within exon 2 (Buchanan et al., Genet. Sel. Evol. (2002) 34:105-116; Nkrumah, J. D. (2004) Can. J. Anim. Sci. (2004) 84:211-219) or the promoter region (Crews, D. H., Can. J. Anim. Sci. (2004) 84:749-750 (Abst.); Nkrumah, J. D. et al., J. Anim. Sci. (2005) 83:20-28) of the leptin gene with carcass and meat quality traits recently were reported in beef cattle.

Polymorphisms in the coding regions of the leptin gene in cattle have been associated with milk yield and composition (see, e.g., Liefers et al., J Dairy Sci. 2002 June; 85(6):1633-8), feed intake (see, e.g., Liefers et al., J Dairy Sci. 2002 June; 85(6):1633-8; Lagonigro et al., Anim Genet. 2003 October; 34(5):371-4), and body fat (see, e.g., Buchanan et al., Genet Sel Evol. January-February; 34(1):105-16; Lagonigro et al., Anim Genet. 2003 October; 34(5):371-4). Polymorphisms in the leptin promoter have been identified, specifically the UASMS1, UASMS2, UASMS3, E2JW, and E2FB SNPs (see, e.g., Nkrumah et al., J Anim Sci. 2005 January; 83(1):20-8; Schenkel et al., J Anim Sci. 2005 September; 83(9):2009-20) and the A59V SNP (see, e.g., Liefers et al., Mamm Genome. 2003 September; 14(9):657-63), however, only the UASM2 SNP (see, e.g., Nkrumah et al., J Anim Sci. 2005 January; 83(1):20-8) has been associated with serum leptin concentration and economically relevant traits of growth, feed intake, efficiency and carcass merit in cattle.

Buchanan et al. (Genet. Sel. Evol. (2002) 34:105-116) identified a cytosine (C) to thymine (T) transition within an exon (exon 2) of the ob gene, corresponding to an arginine (ARG) to cysteine (CYS) substitution in the leptin polypeptide. Exon 2-FB polymorphism is a C/T substitution located at position 305 of exon 2 of the bovine leptin gene according to U50365. The presence of the T-containing allele in bulls was associated with fatter carcasses than those from bulls with the C-containing allele.

SNPs have also been detected in the porcine ob gene and certain of those polymorphisms have been found to be associated with feed intake and carcass traits (Kennes et al. Anim Genet. 2001 August; 32(4):215-8). Means of selective amplification of bovine gene are in U.S. Pat. No. 6,297,027 to Spurlock.

It is possible to distinguish ob genotypes by cloning and sequencing DNA fragments from individual animals, or by other methods known in the art. For example, it is possible to distinguish ob genotypes by employing synthetic oligonucleotide primed amplification of ob gene fragments followed by restriction endonuclease digestion of the amplified product using a restriction enzyme that cuts such product from different ob alleles into discrete product fragments of differing length. Such discrete product fragments could then be distinguished using electrophoresis in agarose or acrylamide, for example. The ob alleles identified by Buchanan et al. (Genet Sel Evol. 2002 January-February; 34(1): 105-16) were distinguished by such means using a mismatch PCR-RFLP strategy wherein, the C-containing allele yields DNA fragments of 75 and 19 bp following digestion of the amplimer with Kpn 21, and the T-containing allele (as above) is not cut.

Accounts on heritabilities and correlations among body condition scores, production traits, and reproductive performance, report that genetic correlation estimates between BCS and production were similar to other estimates in direction and magnitude (Gallo et al., Dairy Sci. 2001 October; 84(10):2321-6; Gallo et al., J Dairy Sci. 1996 June; 79(6):1009-15; Veerkamp et al., J Dairy Sci. 2002 April; 85(4):976-831; Veerkamp et al., J Dairy Sci. 2001 October; 84(10):2327-35 and Veerkamp, J Dairy Sci. 1998 April; 81(4):1109-19). Dechow et al. further report that “Cows that are genetically low for BCS may not maintain energy levels that are sufficient to activate ovarian function or display estrus.” Dechow et al. have also reported on heritability and correlations for body condition score and dairy form within and across lactation and age. (Dechow et al., J Dairy Sci. 2001 January; 84(1):266-75).

Published predicted transmitting abilities (PTA's) for productive life (PL) have been computed using approximate multi-trait methods since July 1994. Evaluations of PL are based on direct observations of length of productive life and also correlated traits measured earlier in life. Multi-trait PTA's have higher reliability (REL) than single-trait PTA's, which include only culling observations. New programs were introduced in August 2000 by the Animal Improvement Programs Laboratory (AIPL) to combine direct and indirect information about longevity. Evaluations of milk, fat, and protein yields; somatic cell score (SCS); and udder, feet and legs (F&L), and body size composites were included in the multi-trait predictions.

The traits and correlations included in multi-trait PL were extended to include stillbirths in August 2006 and daughter pregnancy rate (DPR), service sire calving ease (SCE), and daughter calving ease (DCE) in November 2003. The four calving ease and stillbirth traits are combined before use in PL predictions, and DPR is the most important individual trait in predicting PL. Correlation estimates were revised in November 2000, August 2002, and August 2006. In previous revisions, the influence of yield traits in PL predictions was reduced because correlations of yield traits with PL have decreased over time. In November 2000, foreign bulls received credit for correlated yield and type traits instead of just parent average (PA) or breed average for PL. In May 2001, Interbull somatic cell score evaluations became available and were used as another correlated trait to increase the reliability of estimated PL for foreign bulls.

The multi-trait PTA's for PL begin with single-trait evaluations, which have been calculated at AIPL since January 1994 for all breeds. The single-trait PL evaluations for Holstein bulls were combined with information from yield and type PTA's by Holstein Association USA beginning in July 1994 to produce approximate multi-trait PTA's for PL. Advantages of the multi-trait analysis are greatest when daughters are in first lactation because yield and type data arrive before culling information. The single-trait methods were described by VanRaden and Klaaskate (1993, Journal of Dairy Science 76:2758) and VanRaden and Wiggans (1995, Journal of Dairy Science 78:631). The Holstein Association multi-trait methods were described by Weigel (1996, International Bull Evaluation Service Bulletin 12:125) and Weigel et al. (1998, Journal of Dairy Science 81:2040).

New computer programs were introduced to combine single-trait PTA PL into approximate multi-trait PTA PL (VanRaden, 2001, Journal of Dairy Science 84:E47-E55) beginning with August 2000 evaluations. Udder, feet and legs, and body size composites (Holstein Association USA, 2000, Holstein Type-Production Sire Summaries, August, p. 12-13) were provided to AIPL before evaluation release day for use in the calculation of economic indexes for lifetime net merit. Because of the earlier availability of type information, calculation of multi-trait evaluations for PL was transferred from Holstein Association USA to AIPL. Major differences from the Holstein Association programs were that PTA SCS was included in PL predictions and that PL predictions begin with PA and only adjust the Mendelian sampling for information from correlated traits. Other differences were that the udder, F&L, and body size composites are used in PL prediction rather than all 17 individual linear type traits. Also, all three yield traits (milk, fat, and protein) were used in PL prediction instead of just two (milk and fat). Earlier studies excluded SCS and protein because data for those traits were not widely available in earlier years.

Bovine chromosome 14 (BTA14) harbors several quantitative trait loci (QTL) that affect intramuscular fat (marbling) (see, e.g., Casas et al., J Anim Sci. 2000 March; 78(3):560-9) and subcutaneous fat depth (SFD) (see, e.g., Moore et al., J Anim Sci. 2003 August; 81(8):1919-25) in beef cattle. Recent studies have implicated the corticotrophin-releasing hormone (CRH) gene product in enabling mobilization of energy to cope with stress by stimulating hepatic gluconeogenesis, thus, influencing fat metabolism. Functional studies of the CRH gene in other species (including in mouse (see, e.g., Stenzel-Poore et al., Endocrinology. 1992 June; 130(6):3378-86) and swine (see, e.g., Seasholtz et al., J. Endocrinol. 2002 October; 175(1):89-97) have suggested that CRH is highly associated with body composition (protein and lipid metabolism). The bovine CRH gene is located on BTA14 (see, e.g., Barendse et al., Mamm Genome. 1997 January; 8(1):21-8).

CRH is a growth inhibitor causing the release of glucocorticoids that in turn stimulate the production of both pro-opiomelancortin (POMC) and leptin, which are highly associated with obesity in mammals. Additionally, CRH is most known as a stress hormone. Stress stimulates hepatic gluconeogenesis that will influence fat and protein metabolism in peripheral tissue of animals. For example, a recent study on a porcine CRH gene showed that it functions as a major regulator of neuroendocrine response to stress. It mobilizes energy to cope with stress by stimulating hepatic gluconeogenesis and influencing fat metabolism. Therefore, CRH has a high impact in regulating energy homeostasis, and consequently, it affects body composition (fat deposition) and growth (see, e.g., Murani et al., Biochem Biophys Res Commun. 2006 Apr. 7; 342(2):394-405).

Mitochondrial transcription factor A (“TFAM”), a member of a high mobility group protein family and the first-identified mitochondrial transcription factor (Fisher and Clayton, Mol Cell Biol. 1988; 8:3496-509), is essential for maintenance and biogenesis of mitochondrial DNA (mtDNA). First, TFAM plays a histone-like role in mitochondria, as it is tightly associated with mtDNA as a main component of the nucleoid (Kanki et al. Mol Cell Biol. 2004; 24:9823-34). Evidence has shown that one molecule of mtDNA is packed with 900 molecules of TFAM on average (Alam et al. Nucleic Acids Res. 2003; 31:1640-5), which makes mtDNA no longer naked. Second, TFAM regulates mtDNA copy number in mammals. Investigation using a combination of mice with TFAM overexpression and TFAM knockout demonstrated that mtDNA copy number is directly proportional to the total TFAM protein level in mouse embryos (Ekstrand et al. Hum Mol Genet. 2004; 13:935-44). RNA interference of the endogenous TFAM expression in HeLa cells also indicated that the mtDNA amount is correlated in parallel with the amount of TFAM (Kanki et al. Ann N Y Acad Sci. 2004; 1011:61-8). Third, TFAM stimulates transcription of mtDNA. The TFAM protein possesses two tandem high mobility group domains, which makes TFAM bind, unwind and bend DNA without sequence specificity and thus facilitate transcription initiation of mtDNA (Gaspari et al. 2004; 1659:148-52). Evidence has shown that import of wt-TFAM into liver mitochondria from hypothyroid rats increased RNA synthesis significantly up to 4-fold (Garstka et al. Nucleic Acids Res. 2003; 31:5039-47).

It remains advantageous to provide further SNPs that may more accurately predict the meat quality phenotype of an animal and also a business method that provides for increased production efficiencies in livestock cattle, as well as providing access to various records of the animals and allows comparisons with expected or desired goals with regard to the quality and quantity of animals produced.

Citation or identification of any document in this application is not an admission that such document is available as prior art to the present invention.

### SUMMARY OF THE INVENTION

In the present invention, it has surprisingly been shown that two SNPs located in the promoter of leptin gene promoter and one SNP located in exon 2 are strongly associated with the economically important trait of dairy form in cattle. Dairy form estimates are important for a number of reasons including correlations with other traits.

The present invention relates generally to single nucleotide polymorphisms (SNPs) in the leptin or ob gene and to the association of each of these SNPs with dairy form, a trait of significant economic importance in livestock species. Advantageously, the SNPs are in the promoter of the leptin or ob gene (SEQ ID NO: 2 and SEQ ID NO: 3), or in exon 2 of ob gene (SEQ ID NO: 5). The two SNPs located in the leptin gene promoter are named UASMS1 and UASMS2. These two SNPs, in the context of the ob gene promoter sequence, are in SEQ ID NO: 2, and SEQ ID NO: 3 respectively. The SNP located in exon 2 of the leptin gene is named EXON2-FB, and is in the context of exon 2 of the ob gene in SEQ ID NO: 5.

In one aspect the present invention provides methods for grouping animals according to genotype wherein the animals of each sub-group have a similar polymorphism in the leptin gene. Such methods may comprise determining the genotype of each animal to be subgrouped by determining the presence of a SNP in the leptin gene, wherein the SNP may be selected from the group consisting of UASMS1, UASMS2, and EXON2-FB, and wherein individual animals may be placed into sub-groups where each animal in a subgroup has a similar polymorphism in the leptin gene. In a preferred embodiment the animal to be grouped is a bovine, and the leptin gene is the bovine leptin gene.

In another embodiment, the present invention provides methods for identifying animals having desirable traits relating to milk production, body condition score (BCS), reproductive performance, daughter pregnancy rate (DPR), days open, productive life (PL), and disease incidence (metabolic, mastitis, reproductive, feet and legs), as compared to the general population of animals of that species. Such methods may comprise determining the presence of a SNP in the leptin gene of the animal, wherein the polymorphism is selected from the group consisting of UASMS1, UASMS2, and EXON2-FB, and wherein the presence of either the UASMS1, UASMS2, or EXON2-FB SNP may be indicative of dairy form and thereby of a desirable trait relating to milk production, body condition score (BCS), reproductive performance, daughter pregnancy rate, days open, productive life, and disease incidence (metabolic, mastitis, reproductive, feet and legs), as compared to the general population of animals of that species. In a preferred embodiment the animal to be grouped is a bovine, and the leptin gene is the bovine leptin gene.

In a further embodiment the present invention provides isolated oligonucleotide probes that are useful in the detection of the UASMS1, UASMS2, and EXON2-FB SNPs in the ob gene. The present invention advantageously provides oligonucleotide probes for detection of the two alternative alleles of each SNP. For example, in the case of the UASMS1 polymorphism, which constitutes a C to T substitution at nucleotide position 207 of the ob gene promoter, the present invention provides oligonucleotide probes that may be used to detect and distinguish between the C-containing allele and the T-containing allele. In the case of the UASMS2 polymorphism, which constitutes a C to T substitution at nucleotide position 528 of the ob gene promoter, the present invention provides oligonucleotide probes that may be used to detect and distinguish between the C-containing allele and the T-containing allele. Similarly, in the case of the EXON2-FB polymorphism, which constitutes a C to T substitution at nucleotide position 305 of exon 2 of the ob gene, the present invention provides oligonucleotide probes that may be used to detect and distinguish between the C-containing allele and the T-containing allele. In a preferred embodiment, the oligonucleotide probes of the present invention are labeled with a detectable moiety, such as for example, digoxigenin-dUTP, biotin, fluorescent moieties, chemiluminescent moieties, electrochemiluminescent moieties and radioactive moieties.

In a further embodiment the present invention provides isolated primers and primer pairs that are useful in the amplification of fragments of the ob gene that span the UASMS1, UASMS2, and EXON2-FB SNPs. In one embodiment fragments of the ob gene that are amplified using such primers are subsequently detected using the oligonucleotide probes of the present invention.

The oligonucleotide probes and primers described herein are useful for identifying animals having SNPs associated with desirable traits relating to dairy form, as compared to the general population of animals of that species. Once individual animals possessing these SNPs have been identified, the animals can then be grouped according to genotype, wherein the animals of each sub-group have a similar polymorphism in the leptin gene. The present invention also advantageously provides compositions and kits comprising the oligonucleotide probes and primers described herein.

The present invention relates to the identification of genetic markers (single nucleotide polymorphisms (SNPs)) within a bovine gene encoding leptin, corticotropin-releasing hormone (CRH) and/or mitochondrial transcription factor A (“TFAM”) and their associations with economically relevant traits in beef cattle production, advantageously predicted transmitting abilities (PTA's) for productive life (PL).

The invention encompasses a method for sub-grouping animals according to genotype wherein the animals of each sub-group have similar polymorphisms in a leptin, CRH and/or TFAM gene which may comprise determining the genotype of each animal to be sub-grouped by determining the presence of SNP's in a leptin, CRH and/or TFAM gene, and segregating individual animals into sub-groups wherein each animal in a sub-group has similar polymorphisms in a leptin, CRH and/or TFAM gene.

The invention also encompasses a method for sub-grouping animals according to genotype wherein the animals of each sub-group have a similar genotype in a leptin, CRH and/or TFAM gene which may comprise determining the genotype of each animal to be sub-grouped by determining the presence of a single nucleotide polymorphism(s) of interest in a leptin, CRH and/or TFAM gene, and segregating individual animals into sub-groups depending on whether the animals have, or do not have, the single nucleotide polymorphism(s) of interest in a leptin, CRH and/or TFAM gene.

The leptin genetic polymorphism of interest may be A1457G (position 1540 of SEQ ID NO. 1). The CRH genetic polymorphism(s) of interest may be selected from the group consisting of AAFC03076794.1:g.9657C>T, c. 10718G>C, c. 10841G>A, c. 10893A>C and c. 10936G>C. The TFAM genetic nucleotide polymorphism(s) of interest may be selected from the group consisting of an A to C substitution at the −1220 nucleotide position in the promoter of the TFAM gene, a T to C substitution at position −1212 in the promoter of the TFAM gene and a T to C substitution at position −995 in the promoter of the TFAM gene.

The invention further relates to a method for sub-grouping animals according to genotype wherein the animals of each sub-group have a similar genotype in a leptin, CRH and/or TFAM gene which may comprise determining the genotype of each animal to be sub-grouped by determining the presence of one or more of the above SNPs, and segregating individual animals into sub-groups depending on whether the animals have, or do not have, the above SNPs in a leptin, CRH and/or TFAM gene.

The invention also relates to method for identifying an animal having a desirable phenotype as compared to the general population of animals of that species, which may comprise determining the presence of a single nucleotide polymorphism in a leptin, CRH and/or TFAM gene of the animal, wherein the presence of the SNP is indicative of a desirable phenotype.

In an advantageous embodiment, the animal may be a bovine. In another advantageous embodiment, a leptin, CRH and/or TFAM gene may be a bovine leptin, CRH and/or TFAM gene.

The invention also encompasses computer-assisted methods and systems for improving the production efficiency for livestock having desirable PTA for productive life and in particular the genotype of the animals as it relates to leptin, CRH and/or TFAM SNPs. Methods of the invention encompass obtaining a genetic sample from each animal in a herd of livestock, determining the genotype of each animal with respect to specific quality traits as defined by a panel of at least two, advantageously three, more advantageously four single polynucleotide polymorphisms (SNPs), grouping animals with like genotypes, and optionally, further sub-grouping animals based on like phenotypes. Methods of the invention may also encompass obtaining and maintaining data relating to the animals or to herds, their husbandry conditions, health and veterinary care and condition, genetic history or parentage, and providing this data to others through systems that are web-based, contained in a database, or attached to the animal itself such as by an implanted microchip. An advantageous aspect of the present invention, therefore, is directed to a computer system and computer-assisted methods for tracking quality traits for livestock possessing specific genetic predispositions.

The present invention advantageously encompasses computer-assisted methods and systems for acquiring genetic data, particularly genetic data as defined by the absence or presence of a SNP within a leptin, CRH and/or TFAM gene related to subcutaneous fat traits of the breed of animal and associating those data with other data about the animal or its herd, and maintaining those data in ways that are accessible. Another aspect of the invention encompasses a computer-assisted method for predicting which livestock animals possess a biological difference in PTA for productive life, and which may include the steps of using a computer system, e.g., a programmed computer comprising a processor, a data storage system, an input device and an output device, the steps of: (a) inputting into the programmed computer through the input device data that includes a genotype of an animal as it relates to any one of the leptin, CRH and/or TFAM SNPs described herein, (b) correlating PTA for productive life predicted by the leptin, CRH and/or TFAM genotype using the processor and the data storage system and (c) outputting to the output device the PTA for productive life correlated to the leptin, CRH and/or TFAM genotype, thereby predicting which livestock animals possess a particular PTA for productive life.

Yet another aspect of the invention relates to a method of doing business for managing livestock comprising providing to a user computer system for managing livestock comprising physical characteristics and genotypes corresponding to one or more animals or a computer readable media for managing livestock comprising physical characteristics and genotypes corresponding to one or more animals or physical characteristics and genotypes corresponding to one or more animals, wherein a physical characteristic intake, growth or carcass merit in beef cattle and the genotype is a leptin, CRH and/or TFAM genotype.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1 illustrates the nucleotide sequence of the 5′ flanking promoter region and exon 1 of the “wild type” bovine ob gene. This “wild type” sequence has GenBank accession number AB070368 (Taniguchi et al. IUBMB Life Vol 53, p 131-135 (2002)), and is designated herein as SEQ ID NO. 1.

FIG. 2 illustrates the nucleotide sequence the UASMS1 single nucleotide polymorphism in the bovine ob gene promoter (SEQ ID NO. 2). This polymophic sequence differs from that of the “wild type” bovine ob gene sequence (SEQ ID NO. 1) in that nucleotide position 207 has a cytosine to thymine substitution.

FIG. 3 illustrates the nucleotide sequence the UASMS2 single nucleotide polymorphism of the bovine ob gene (SEQ ID NO. 3). This polymophic sequence differs from that of the “wild type” bovine ob gene sequence (SEQ ID NO. 1) in that nucleotide position 528 has a cytosine to thymine substitution.

FIG. 4 illustrates the nucleotide sequence the exon 2 of the “wild type” bovine ob gene (SEQ ID NO. 5). This “wild type” exon 2 sequence has GenBank accession number AY138588.

FIG. 5 illustrates the nucleotide sequence the EXON2-FB single nucleotide polymorphism of the bovine ob gene (SEQ ID NO. 6). This polymophic sequence differs from that of the “wild type” bovine ob gene sequence (SEQ ID NO. 5) in that nucleotide position 305 has a cytosine to thymine substitution.

FIG. 6 depicts the leptin haplotype combinations for 300 leading AI sires and their associations with the trait of productive life.

FIG. 7 depicts the leptin haplotype combinations for 300 leading AI sires and their associations with the trait of productive life.

FIG. 8 depicts the leptin haplotype combinations for 300 leading AI sires and their associations with the trait of dairy form.

FIG. 9 depicts the leptin haplotype combinations for 300 leading AI sires and their associations with the trait of dairy form.

FIG. 10 depicts the leptin haplotype combinations for 300 leading AI sires and their associations with the trait of dairy form.

FIG. 11 depicts the leptin haplotype combinations for 300 leading AI sires and their associations with the trait of productive life.

FIG. 12 illustrates a flowchart of the input of data and the output of results from the analysis and correlation of the data pertaining to the breeding, veterinarian histories and performance requirements of a group of animals such as from a herd of cows and the interactive flow of data from the computer-assisted device to a body of students learning the use of the method of the invention.

FIG. 13 illustrates potential relationships between the data elements to be entered into the system. Unidirectional arrows indicate, for example, that a house or shed is typically owned by only one farm, whereas a farm may own several houses or sheds. Similarly, a prescription may include have several veterinarian products.

FIG. 14A illustrates the flow of events in the use of the portable computer-based system for data entry on the breeding and rearing of a herd of cows.

FIG. 14B illustrates the flow of events through the sub-routines related to data entry concerning farm management.

FIG. 14C illustrates the flow of events through the sub-routines related to data entry concerning data specific to a company.

FIG. 15 illustrates a flow chart of the input of data and the output of results from the analysis and the correlation of the data pertaining to the breeding, veterinarian histories, and performance requirements of a group of animals.

FIG. 16 illustrates the nucleotide sequence of the forward primer used for UASMS1 polymorphism amplification

FIG. 17 illustrates the nucleotide sequence of the reverse primer used for UASMS1 polymorphism amplification.

FIG. 18 illustrates the nucleotide sequence of the forward primer used for UASMS2 polymorphism amplification.

FIG. 19 illustrates the nucleotide sequence of the reverse primer used for UASMS2 polymorphism amplification.

FIG. 20 illustrates the nucleotide sequence of the forward primer used for EXON2-FB polymorphism amplification.

FIG. 21 illustrates the nucleotide sequence of the reverse primer used for EXON2-FB polymorphism amplification.

FIG. 22 illustrates the nucleotide sequence of the T-containing allele-specific probe used for UASMS1 ob polymorphism detection.

FIG. 23 illustrates the nucleotide sequence of the C-containing allele-specific probe used for UASMS1 ob polymorphism detection.

FIG. 24 illustrates the nucleotide sequence of the T-containing allele-specific probe used for UASMS2 ob polymorphism detection.

FIG. 25 illustrates the nucleotide sequence of the C-containing allele-specific probe used for UASMS2 ob polymorphism detection.

FIG. 26 illustrates the nucleotide sequence of the T-containing allele-specific probe used for EXON2-FB ob polymorphism detection.

FIG. 27 illustrates the nucleotide sequence of the C-containing allele-specific probe used for EXON2-FB ob polymorphism detection.

FIG. 28 depicts the different in PTA productive life between bulls with different scores.

FIG. 29 illustrates the nucleotide sequences of the forward and reverse primers used for CRH, leptin A1457G and TFAM polymorphism amplification.

### DETAILED DESCRIPTION

The term “animal” is used herein to include all vertebrate animals, including humans. It also includes an individual animal in all stages of development, including embryonic and fetal stages. As used herein, the term “production animals” is used interchangeably with “livestock animals” and refers generally to animals raised primarily for food. For example, such animals include, but are not limited to, cattle (bovine), sheep (ovine), pigs (porcine or swine), poultry (avian), and the like. As used herein, the term “cow” or “cattle” is used generally to refer to an animal of bovine origin of any age. Interchangeable terms include “bovine”, “calf”, “steer”, “bull”, “heifer”, “cow” and the like. As used herein, the term “pig” is used generally to refer to an animal of porcine origin of any age. Interchangeable terms include “piglet”, “sow” and the like.

By the term “complementarity” or “complementary” is meant, for the purposes of the specification or claims, a sufficient number in the oligonucleotide of complementary base pairs in its sequence to interact specifically (hybridize) with the target nucleic acid sequence of the ob gene polymorphism to be amplified or detected. As known to those skilled in the art, a very high degree of complementarity is needed for specificity and sensitivity involving hybridization, although it need not be 100%. Thus, for example, an oligonucleotide that is identical in nucleotide sequence to an oligonucleotide disclosed herein, except for one base change or substitution, may function equivalently to the disclosed oligonucleotides. A “complementary DNA” or “cDNA” gene includes recombinant genes synthesized by reverse transcription of messenger RNA (“mRNA”).

A “cyclic polymerase-mediated reaction” refers to a biochemical reaction in which a template molecule or a population of template molecules is periodically and repeatedly copied to create a complementary template molecule or complementary template molecules, thereby increasing the number of the template molecules over time.

“Denaturation” of a template molecule refers to the unfolding or other alteration of the structure of a template so as to make the template accessible to duplication. In the case of DNA, “denaturation” refers to the separation of the two complementary strands of the double helix, thereby creating two complementary, single stranded template molecules. “Denaturation” can be accomplished in any of a variety of ways, including by heat or by treatment of the DNA with a base or other denaturant.

A “detectable amount of product” refers to an amount of amplified nucleic acid that can be detected using standard laboratory tools. A “detectable marker” refers to a nucleotide analog that allows detection using visual or other means. For example, fluorescently labeled nucleotides can be incorporated into a nucleic acid during one or more steps of a cyclic polymerase-mediated reaction, thereby allowing the detection of the product of the reaction using, e.g. fluorescence microscopy or other fluorescence-detection instrumentation.

By the term “detectable moiety” is meant, for the purposes of the specification or claims, a label molecule (isotopic or non-isotopic) which is incorporated indirectly or directly into an oligonucleotide, wherein the label molecule facilitates the detection of the oligonucleotide in which it is incorporated, for example when the oligonucleotide is hybridized to amplified ob gene polymorphisms sequences. Thus, “detectable moiety” is used synonymously with “label molecule”. Synthesis of oligonucleotides can be accomplished by any one of several methods known to those skilled in the art. Label molecules, known to those skilled in the art as being useful for detection, include chemiluminescent or fluorescent molecules. Various fluorescent molecules are known in the art which are suitable for use to label a nucleic acid for the method of the present invention. The protocol for such incorporation may vary depending upon the fluorescent molecule used. Such protocols are known in the art for the respective fluorescent molecule.

By “detectably labeled” is meant that a fragment or an oligonucleotide contains a nucleotide that is radioactive, or that is substituted with a fluorophore, or that is substituted with some other molecular species that elicits a physical or chemical response that can be observed or detected by the naked eye or by means of instrumentation such as, without limitation, scintillation counters, calorimeters, UV spectrophotometers and the like. As used herein, a “label” or “tag” refers to a molecule that, when appended by, for example, without limitation, covalent bonding or hybridization, to another molecule, for example, also without limitation, a polynucleotide or polynucleotide fragment, provides or enhances a means of detecting the other molecule. A fluorescence or fluorescent label or tag emits detectable light at a particular wavelength when excited at a different wavelength. A radiolabel or radioactive tag emits radioactive particles detectable with an instrument such as, without limitation, a scintillation counter. Other signal generation detection methods include: chemiluminescence, electrochemiluminescence, raman, calorimetric, hybridization protection assay, and mass spectrometry

“DNA amplification” as used herein refers to any process that increases the number of copies of a specific DNA sequence by enzymatically amplifying the nucleic acid sequence. A variety of processes are known. One of the most commonly used is the polymerase chain reaction (PCR), which is defined and described in later sections below. The PCR process of Mullis is described in U.S. Pat. Nos. 4,683,195 and 4,683,202. PCR involves the use of a thermostable DNA polymerase, known sequences as primers, and heating cycles, which separate the replicating deoxyribonucleic acid (DNA), strands and exponentially amplify a gene of interest. Any type of PCR, such as quantitative PCR, RT-PCR, hot start PCR, LAPCR, multiplex PCR, touchdown PCR, etc., may be used. Advantageously, real-time PCR is used. In general, the PCR amplification process involves an enzymatic chain reaction for preparing exponential quantities of a specific nucleic acid sequence. It requires a small amount of a sequence to initiate the chain reaction and oligonucleotide primers that will hybridize to the sequence. In PCR the primers are annealed to denatured nucleic acid followed by extension with an inducing agent (enzyme) and nucleotides. This results in newly synthesized extension products. Since these newly synthesized sequences become templates for the primers, repeated cycles of denaturing, primer annealing, and extension results in exponential accumulation of the specific sequence being amplified. The extension product of the chain reaction will be a discrete nucleic acid duplex with a termini corresponding to the ends of the specific primers employed. “DNA” refers to the polymeric form of deoxyribonucleotides (adenine, guanine, thymine, or cytosine) in either single stranded form, or as a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

By the terms “enzymatically amplify” or “amplify” is meant, for the purposes of the specification or claims, DNA amplification, i.e., a process by which nucleic acid sequences are amplified in number. There are several means for enzymatically amplifying nucleic acid sequences. Currently the most commonly used method is the polymerase chain reaction (PCR). Other amplification methods include LCR (ligase chain reaction) which utilizes DNA ligase, and a probe consisting of two halves of a DNA segment that is complementary to the sequence of the DNA to be amplified, enzyme QB replicase and a ribonucleic acid (RNA) sequence template attached to a probe complementary to the DNA to be copied which is used to make a DNA template for exponential production of complementary RNA; strand displacement amplification (SDA); Q.beta. replicase amplification (Q.beta.RA); self-sustained replication (3SR); and NASBA (nucleic acid sequence-based amplification), which can be performed on RNA or DNA as the nucleic acid sequence to be amplified.

A “fragment” of a molecule such as a protein or nucleic acid is meant to refer to any portion of the amino acid or nucleotide genetic sequence.

As used herein, the term “genome” refers to all the genetic material in the chromosomes of a particular organism. Its size is generally given as its total number of base pairs. Within the genome, the term “gene” refers to an ordered sequence of nucleotides located in a particular position on a particular chromosome that encodes a specific functional product (e.g., a protein or RNA molecule). For example, it is known that the protein leptin is encoded by the ob (obese) gene and appears to be involved in the regulation of appetite, basal metabolism and fat deposition. In general, an animal's genetic characteristics, as defined by the nucleotide sequence of its genome, are known as its “genotype,” while the animal's physical traits are described as its “phenotype.”

By “heterozygous” or “heterozygous polymorphism” is meant that the two alleles of a diploid cell or organism at a given locus are different, that is, that they have a different nucleotide exchanged for the same nucleotide at the same place in their sequences.

By “homozygous” or “homozygous polymorphism” is meant that the two alleles of a diploid cell or organism at a given locus are identical, that is, that they have the same nucleotide for nucleotide exchange at the same place in their sequences.

By “hybridization” or “hybridizing,” as used herein, is meant the formation of A-T and C-G base pairs between the nucleotide sequence of a fragment of a segment of a polynucleotide and a complementary nucleotide sequence of an oligonucleotide. By complementary is meant that at the locus of each A, C, G or T (or U in a ribonucleotide) in the fragment sequence, the oligonucleotide sequenced has a T, G, C or A, respectively. The hybridized fragment/oligonucleotide is called a “duplex.”

A “hybridization complex”, such as in a sandwich assay, means a complex of nucleic acid molecules including at least the target nucleic acid and a sensor probe. It may also include an anchor probe.

By “immobilized on a solid support” is meant that a fragment, primer or oligonucleotide is attached to a substance at a particular location in such a manner that the system containing the immobilized fragment, primer or oligonucleotide may be subjected to washing or other physical or chemical manipulation without being dislodged from that location. A number of solid supports and means of immobilizing nucleotide-containing molecules to them are known in the art; any of these supports and means may be used in the methods of this invention.

As used herein, the term “increased weight gain” means a biologically significant increase in weight gain above the mean of a given population.

As used herein, the term “locus” or “loci” refers to the site of a gene on a chromosome. A single allele from each locus is inherited from each parent. Each animal's particular combination of alleles is referred to as its “genotype”. Where both alleles are identical, the individual is said to be homozygous for the trait controlled by that pair of alleles; where the alleles are different, the individual is said to be heterozygous for the trait.

A “melting temperature” is meant the temperature at which hybridized duplexes dehybridize and return to their single-stranded state. Likewise, hybridization will not occur in the first place between two oligonucleotides, or, herein, an oligonucleotide and a fragment, at temperatures above the melting temperature of the resulting duplex. It is presently advantageous that the difference in melting point temperatures of oligonucleotide-fragment duplexes of this invention be from about 1 C to about 10 C so as to be readily detectable.

As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but advantageously is double-stranded DNA. An “isolated” nucleic acid molecule is one that is separated from other nucleic acid molecules that are present in the natural source of the nucleic acid. A “nucleoside” refers to a base linked to a sugar. The base may be adenine (A), guanine (G) (or its substitute, inosine (I)), cytosine (C), or thymine (T) (or its substitute, uracil (U)). The sugar may be ribose (the sugar of a natural nucleotide in RNA) or 2-deoxyribose (the sugar of a natural nucleotide in DNA). A “nucleotide” refers to a nucleoside linked to a single phosphate group.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides may be chemically synthesized and may be used as primers or probes. Oligonucleotide means any nucleotide of more than 3 bases in length used to facilitate detection or identification of a target nucleic acid, including probes and primers.

“Polymerase chain reaction” or “PCR” refers to a thermocyclic, polymerase-mediated, DNA amplification reaction. A PCR typically includes template molecules, oligonucleotide primers complementary to each strand of the template molecules, a thermostable DNA polymerase, and deoxyribonucleotides, and involves three distinct processes that are multiply repeated to effect the amplification of the original nucleic acid. The three processes (denaturation, hybridization, and primer extension) are often performed at distinct temperatures, and in distinct temporal steps. In many embodiments, however, the hybridization and primer extension processes can be performed concurrently. The nucleotide sample to be analyzed may be PCR amplification products provided using the rapid cycling techniques described in U.S. Pat. Nos. 6,569,672; 6,569,627; 6,562,298; 6,556,940; 6,569,672; 6,569,627; 6,562,298; 6,556,940; 6,489,112; 6,482,615; 6,472,156; 6,413,766; 6,387,621; 6,300,124; 6,270,723; 6,245,514; 6,232,079; 6,228,634; 6,218,193; 6,210,882; 6,197,520; 6,174,670; 6,132,996; 6,126,899; 6,124,138; 6,074,868; 6,036,923; 5,985,651; 5,958,763; 5,942,432; 5,935,522; 5,897,842; 5,882,918; 5,840,573; 5,795,784; 5,795,547; 5,785,926; 5,783,439; 5,736,106; 5,720,923; 5,720,406; 5,675,700; 5,616,301; 5,576,218 and 5,455,175, the disclosures of which are incorporated by reference in their entireties. Other methods of amplification include, without limitation, NASBR, SDA, 3SR, TSA and rolling circle replication. It is understood that, in any method for producing a polynucleotide containing given modified nucleotides, one or several polymerases or amplification methods may be used. The selection of optimal polymerization conditions depends on the application.

A “polymerase” is an enzyme that catalyzes the sequential addition of monomeric units to a polymeric chain, or links two or more monomeric units to initiate a polymeric chain. In advantageous embodiments of this invention, the “polymerase” will work by adding monomeric units whose identity is determined by and which is complementary to a template molecule of a specific sequence. For example, DNA polymerases such as DNA pol 1 and Taq polymerase add deoxyribonucleotides to the 3′ end of a polynucleotide chain in a template-dependent manner, thereby synthesizing a nucleic acid that is complementary to the template molecule. Polymerases may be used either to extend a primer once or repetitively or to amplify a polynucleotide by repetitive priming of two complementary strands using two primers.

A “polynucleotide” refers to a linear chain of nucleotides connected by a phosphodiester linkage between the 3′-hydroxyl group of one nucleoside and the 5′-hydroxyl group of a second nucleoside which in turn is linked through its 3′-hydroxyl group to the 5′-hydroxyl group of a third nucleoside and so on to form a polymer comprised of nucleosides liked by a phosphodiester backbone. A “modified polynucleotide” refers to a polynucleotide in which one or more natural nucleotides have been partially or substantially replaced with modified nucleotides.

A “primer” is an oligonucleotide, the sequence of at least a portion of which is complementary to a segment of a template DNA which to be amplified or replicated. Typically primers are used in performing the polymerase chain reaction (PCR). A primer hybridizes with (or “anneals” to) the template DNA and is used by the polymerase enzyme as the starting point for the replication/amplification process. By “complementary” is meant that the nucleotide sequence of a primer is such that the primer can form a stable hydrogen bond complex with the template; i.e., the primer can hybridize or anneal to the template by virtue of the formation of base-pairs over a length of at least ten consecutive base pairs.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

“Probes” refer to oligonucleotides nucleic acid sequences of variable length, used in the detection of identical, similar, or complementary nucleic acid sequences by hybridization. An oligonucleotide sequence used as a detection probe may be labeled with a detectable moiety. Various labeling moieties are known in the art. Said moiety may, for example, either be a radioactive compound, a detectable enzyme (e.g. horse radish peroxidase (HRP)) or any other moiety capable of generating a detectable signal such as a calorimetric, fluorescent, chemiluminescent or electrochemiluminescent signal. The detectable moiety may be detected using known methods.

As used herein, the term “protein” refers to a large molecule composed of one or more chains of amino acids in a specific order. The order is determined by the base sequence of nucleotides in the gene coding for the protein. Proteins are required for the structure, function, and regulation of the body's cells, tissues, and organs. Each protein has a unique function.

As used herein, the terms “quality traits,” “traits,” or “physical characteristics” refer to advantageous properties of the animal resulting from genetics. Quality traits include, but are not limited to, the animal's genetic ability to metabolize energy, produce milk, put on intramuscular fat, lay eggs, produce offspring, produce particular proteins in meat or milk, or retain protein in milk. Physical characteristics include marbled or lean meats. The terms are used interchangeably.

A “restriction enzyme” refers to an endonuclease (an enzyme that cleaves phosphodiester bonds within a polynucleotide chain) that cleaves DNA in response to a recognition site on the DNA. The recognition site (restriction site) consists of a specific sequence of nucleotides typically about 4-8 nucleotides long.

A “single nucleotide polymorphism” or “SNP” refers to polynucleotide that differs from another polynucleotide by a single nucleotide exchange. For example, without limitation, exchanging one A for one C, G, or T in the entire sequence of polynucleotide constitutes a SNP. Of course, it is possible to have more than one SNP in a particular polynucleotide. For example, at one locus in a polynucleotide, a C may be exchanged for a T, at another locus a G may be exchanged for an A, and so on. When referring to SNPs, the polynucleotide is most often DNA.

As used herein, a “template” refers to a target polynucleotide strand, for example, without limitation, an unmodified naturally-occurring DNA strand, which a polymerase uses as a means of recognizing which nucleotide it should next incorporate into a growing strand to polymerize the complement of the naturally-occurring strand. Such DNA strand may be single-stranded or it may be part of a double-stranded DNA template. In applications of the present invention requiring repeated cycles of polymerization, e.g., the polymerase chain reaction (PCR), the template strand itself may become modified by incorporation of modified nucleotides, yet still serve as a template for a polymerase to synthesize additional polynucleotides.

A “thermocyclic reaction” is a multi-step reaction wherein at least two steps are accomplished by changing the temperature of the reaction.

A “thermostable polymerase” refers to a DNA or RNA polymerase enzyme that can withstand extremely high temperatures, such as those approaching 100 C. Often, thermostable polymerases are derived from organisms that live in extreme temperatures, such as Thermus aquaticus. Examples of thermostable polymerases include Taq, Tth, Pfu, Vent, deep vent, UlTma, and variations and derivatives thereof.

A “variance” is a difference in the nucleotide sequence among related polynucleotides. The difference may be the deletion of one or more nucleotides from the sequence of one polynucleotide compared to the sequence of a related polynucleotide, the addition of one or more nucleotides or the substitution of one nucleotide for another. The terms “mutation,” “polymorphism” and “variance” are used interchangeably herein. As used herein, the term “variance” in the singular is to be construed to include multiple variances; i.e., two or more nucleotide additions, deletions and/or substitutions in the same polynucleotide. A “point mutation” refers to a single substitution of one nucleotide for another.

A “computer system” refers to the hardware means, software means and data storage means used to compile the data of the present invention. The minimum hardware means of computer-based systems of the invention may comprise a central processing unit (CPU), input means, output means, and data storage means. Desirably, a monitor is provided to visualize structure data. The data storage means may be RAM or other means for accessing computer readable media of the invention. Examples of such systems are microcomputer workstations available from Silicon Graphics Incorporated and Sun Microsystems running Unix based, Linux, Windows NT, XP or IBM OS/2 operating systems.

“Computer readable media” refers to any media which can be read and accessed directly by a computer, and includes, but is not limited to: magnetic storage media such as floppy discs, hard storage medium and magnetic tape; optical storage media such as optical discs or CD-ROM; electrical storage media such as RAM and ROM; and hybrids of these categories, such as magnetic/optical media. By providing such computer readable media, the data compiled on a particular animal can be routinely accessed by a user, e.g., a feedlot operator.

The term “data analysis module” is defined herein to include any person or machine, individually or working together, which analyzes the sample and determines the genetic information contained therein. The term may include a person or machine within a laboratory setting.

As used herein, the term “data collection module” refers to any person, object or system obtaining a tissue sample from an animal or embryo. By example and without limitation, the term may define, individually or collectively, the person or machine in physical contact with the animal as the sample is taken, the containers holding the tissue samples, the packaging used for transporting the samples, and the like. Advantageously, the data collector is a person. More advantageously, the data collector is a livestock farmer, a breeder or a veterinarian

The term “network interface” is defined herein to include any person or computer system capable of accessing data, depositing data, combining data, analyzing data, searching data, transmitting data or storing data. The term is broadly defined to be a person analyzing the data, the electronic hardware and software systems used in the analysis, the databases storing the data analysis, and any storage media capable of storing the data. Non-limiting examples of network interfaces include people, automated laboratory equipment, computers and computer networks, data storage devices such as, but not limited to, disks, hard drives or memory chips.

The term “breeding history” as used herein refers to a record of the life of an animal or group of animals including, but not limited to, the location, breed, period of housing, as well as a genetic history of the animals, including parentage and descent therefrom, genotype, phenotype, transgenic history if relevant and the like.

The term “husbandry conditions” as used herein refers to parameters relating to the maintenance of animals including, but not limited to, shed or housing temperature, weekly mortality of a herd, water consumption, feed consumption, ventilation rate and quality, litter condition and the like.

The term “veterinary history” as used herein refers to vaccination data of an animal or group of animals, including, but not limited to, vaccine type(s), vaccine batch serial number(s), administered dose, target antigen, method of administering of the vaccine to the recipient animal(s), number of vaccinated animals, age of the animals and the vaccinator. Data relating to a serological or immunological response induced by the vaccine may also be included. “Veterinary history” as used herein is also intended to include the medication histories of the target animal(s) including, but not limited to drug and/or antibiotics administered to the animals including type of administered medication, quantity and dose rates, by whom and when administered, by what route, e.g., oral, subcutaneously and the like, and the response to the medication including desired and undesirable effects thereof.

The term “diagnostic data” as used herein refers to data relating to the health of the animal(s) other than data detailing the vaccination or medication history of the animal(s). For example, the diagnostic data may be a record of the infections experienced by the animal(s) and the response thereof to medications provided to treat such medications. Serological data including antibody or protein composition of the serum or other biofluids may also be diagnostic data useful to input in the methods of the invention. Surgical data pertaining to the animal(s) may be included, such as the type of surgical manipulation, outcome of the surgery and complications arising from the surgical procedure. “Diagnostic data” may also include measurements of such parameters as weight, morbidity, and other characteristics noted by a veterinary service such as the condition of the skin, feet etc.

The term “welfare data” as used herein refers to the collective accumulation of data pertaining to an animal or group of animals including, but not limited to, a breeding history, a veterinary history, a welfare profile, diagnostic data, quality control data, or any combination thereof.

The term “welfare profile” as used herein refers to parameters such as weight, meat density, crowding levels in breeding or rearing enclosures, psychological behavior of the animal, growth rate and quality and the like.

The term “quality control” as used herein refers to the desired characteristics of the animal(s). For non-poultry animals such as cattle and sheep for example, such parameters include muscle quantity and density, fat content, meat tenderness, milk yield and quality, breeding ability, and the like.

The term “performance parameters” as used herein refers to such factors as meat yield, breeding yield, dairy form, meat quality and yield, productive life and the like that may be the desired goals from the breeding and rearing of the animal(s). Performance parameters may be either generated from the animals themselves, or those parameters desired by a customer or the market.

The term “nutritional data” as used herein refers to the composition, quantity and frequency of delivery of feed, including water, provided to the animal(s).

The term “food safety” as used herein refers to the quality and quantity of the meat from a livestock animal, including, but not limited to, preparation time, place and manner, storage of the food product, transportation route, inspection records, texture, color, taste, odor, bacterial content, parasitic content and the like.

It will be apparent to those of skill in the art that the data relating to the health and maintenance of the animals may be variously grouped depending upon the source or intention of the data collector and any one grouping herein is not therefore intended to be limiting.

Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein.

The present invention provides methods for the identification and selection of animals based on the presence of SNPs in the ob (obese) gene—a gene that encodes the protein leptin. Leptin is a 16-kDa adipocyte-specific polypeptide involved in the regulation of appetite, basal metabolism, fat deposition and milk production. The ob gene has been mapped to specific chromosomes in several different animals, allowing the gene to be sequenced in several different species. It has been found that there is significant conservation of ob DNAs and leptin polypeptides between species. SNPs having the same or similar phenotypic effects to those of the present invention may occur in many different animal species. The methods of the present invention can be used to determine whether an individual animal from a species of interest possesses the SNPs described herein. In a preferred embodiment, the ob gene of a bovine animal is screened for the presence of the SNPs of the present invention.

In one aspect, the present invention relates to the identification of single nucleotide polymorphisms (SNPs) in the leptin promoter, and to methods for the identification of animals carrying specific alleles of these SNPs that are associated with dairy form. In a further aspect, the present invention relates the association of a previously reported SNP in exon 2 of the leptin gene, with dairy form. The present invention also provides oligonucleotides that can be used as primers to amplify specific nucleic acid sequences of the ob gene, and oligonucleotides that can be used as probes in the detection of nucleic acid sequences of the ob gene.

Dairy form is a type trait that is determined by U.S. Holstein Association classifiers. Classifiers evaluate each cow and assign them a score of 1 to 50; 1 being low dairy form (thick, round-boned, tight ribbed) and 50 being high dairy form (angular, flat-boned, open ribbed). The average dairy form score in today's Holsteins is 32.4 and the standard deviation is 1.2. As an example, for a bull with a 0.7 STA for dairy form, you would expect his daughters to average 0.7 standard units (0.7*1.2) higher in dairy form when he is bred to average cows in average herds. So, his daughters would score 0.8 point higher than average on dairy form, or 33.2.

The importance of dairy form can be summarized by looking at what has happened in the dairy industry as a result of heavy selection emphasis on high milk yield and, to some extent, high dairy form: high dairy form correlates with high milk production, low body condition score (which correlates with low reproductive performance), low daughter pregnancy rate, high days open, low productive life, and high disease incidence (metabolic, mastitis, reproductive, feet & legs).

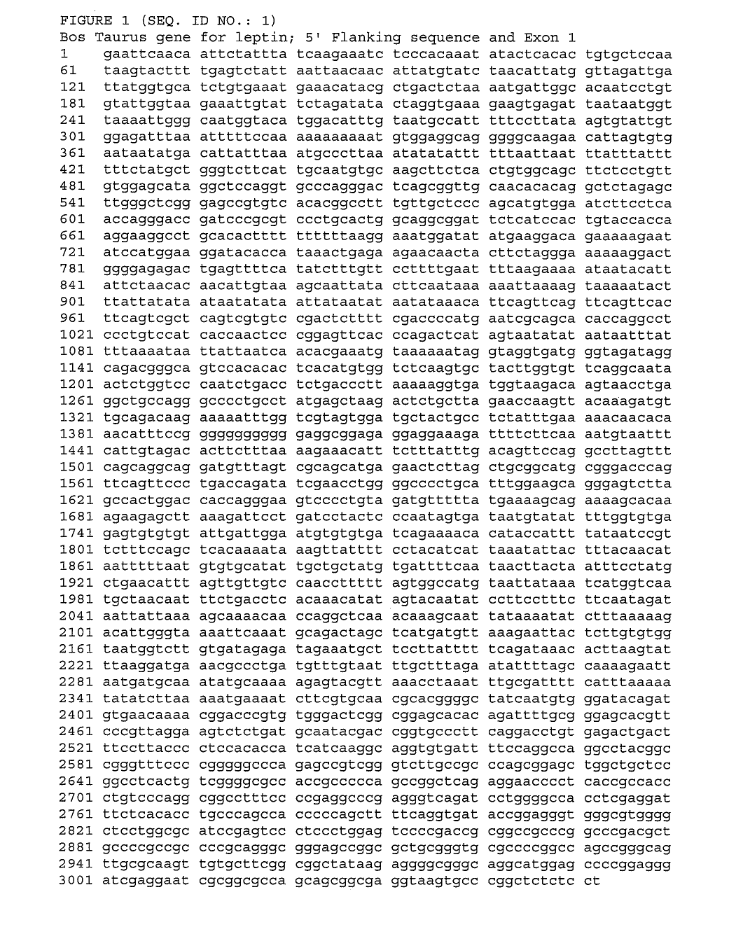

In the present invention it has surprisingly been shown that two SNPs (namely UASMS1 and UASMS2) located in the promoter region of the ob gene, and one SNP in exon 2 of the gene are associated with dairy form in animals, in particular in bovine livestock. FIG. 1 illustrates the nucleotide sequence for the 5′ flanking promoter region and exon 1 of the “wild type” bovine ob gene. This “wild type” sequence has GenBank accession number AB070368, (Taniguchi et al. IUBMB Life Vol 53, p 131-135 (2002)), and is designated herein as SEQ ID NO. 1.

The SNP termed UASMS1 constitutes a cytosine (C) to thymine (T) substitution (C/T) at position 207 of the bovine leptin gene promoter. The SNP termed UASMS2, constitutes a cytosine (C) to thymine (T) substitution (C/T substitution) at position 528 of bovine leptin gene promoter. The nucleotide numbering system used herein for the identification of the leptin promoter SNPs UASMS1 and UASMS2 is that used for the “wild type” bovine leptin promoter sequence SEQ ID NO. 1.

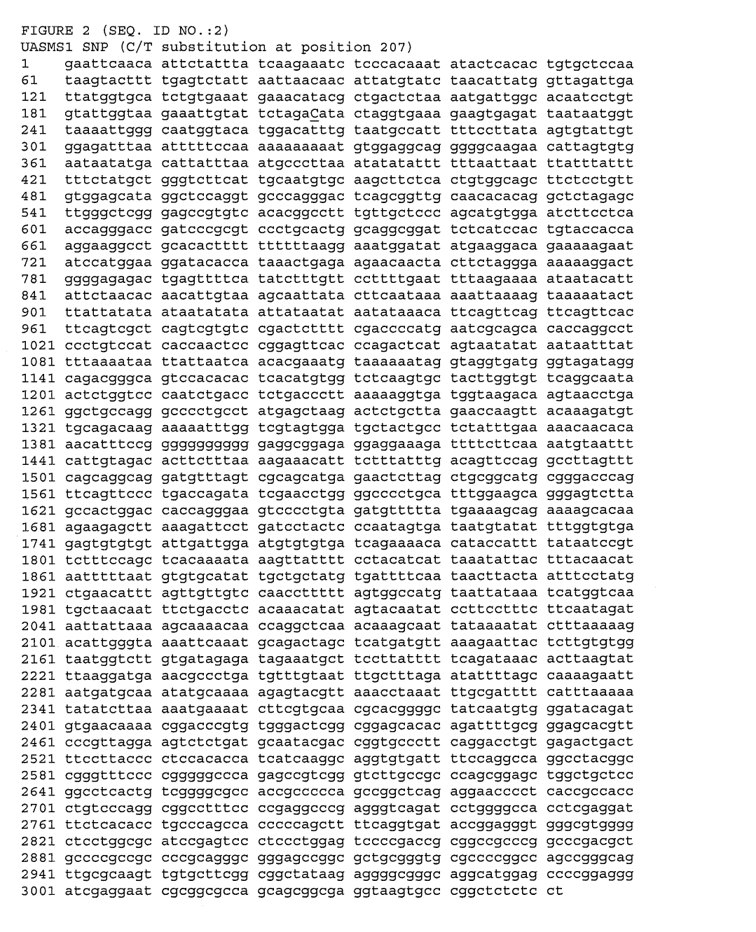

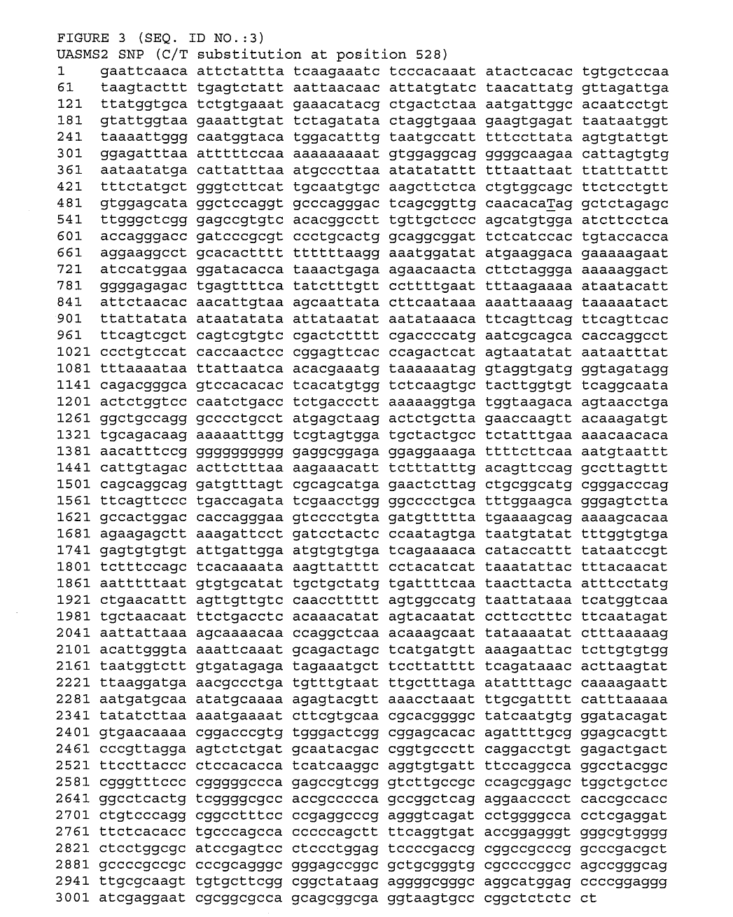

The UASMS1 and UASMS2 polymorphisms are located in the 5′ regulatory sequence of the leptin gene, not the coding region of the gene, and thus do not result in any amino acid substitution in the leptin gene product. FIG. 2 illustrates the nucleotide sequence the UASMS1 single nucleotide polymorphism in the bovine ob gene promoter (SEQ ID NO. 2). This polymophic sequence differs from that of the “wild type” bovine ob gene sequence (SEQ ID NO. 1) in that nucleotide position 207 has a cytosine to thymine substitution. FIG. 3 illustrates the nucleotide sequence the UASMS2 single nucleotide polymorphism of the bovine ob gene (SEQ ID NO. 3). This polymophic sequence differs from that of the “wild type” bovine ob gene sequence (SEQ ID NO. 1) in that nucleotide position 528 has a cytosine to thymine substitution.

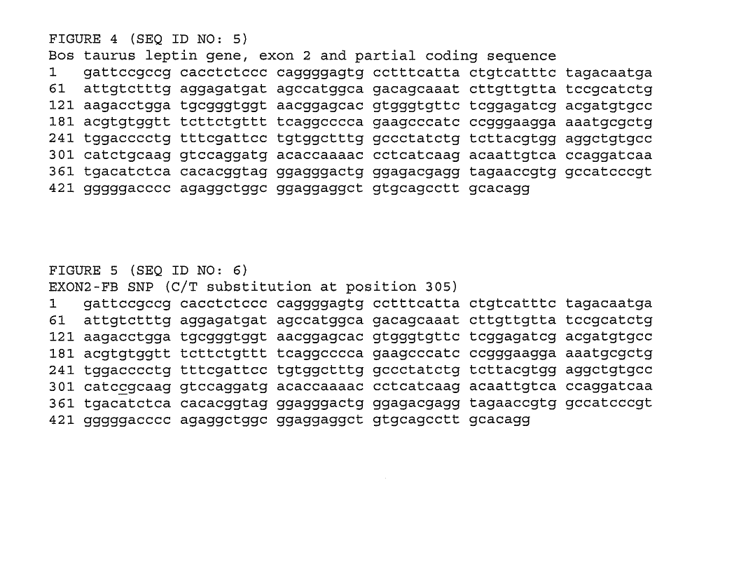

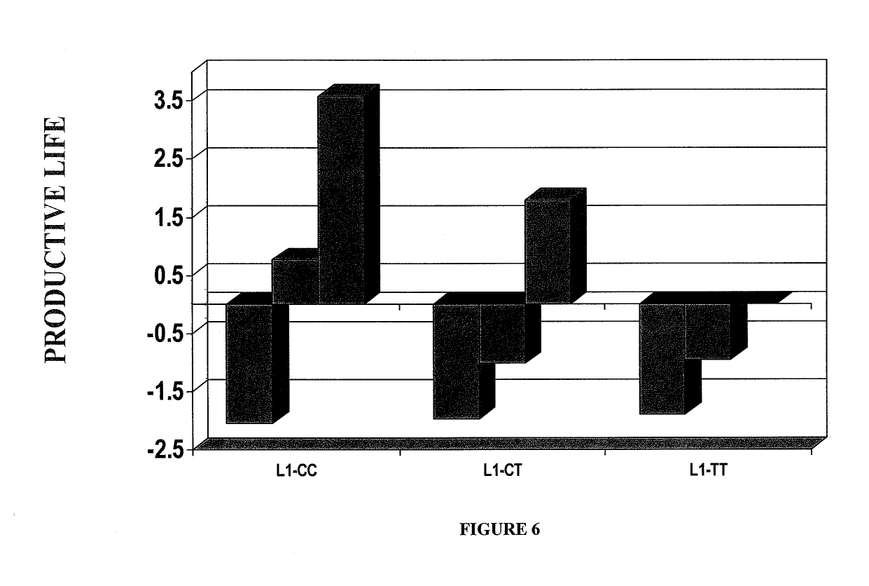

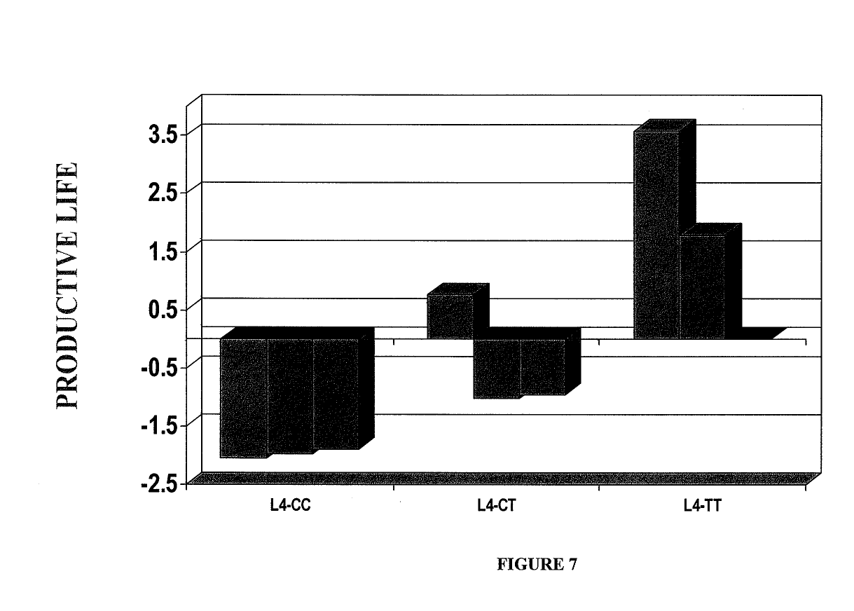

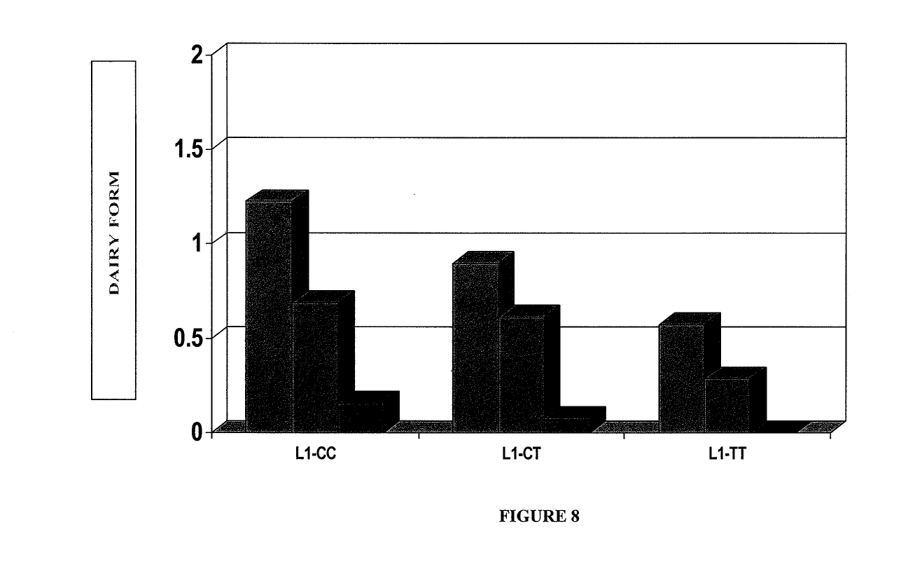

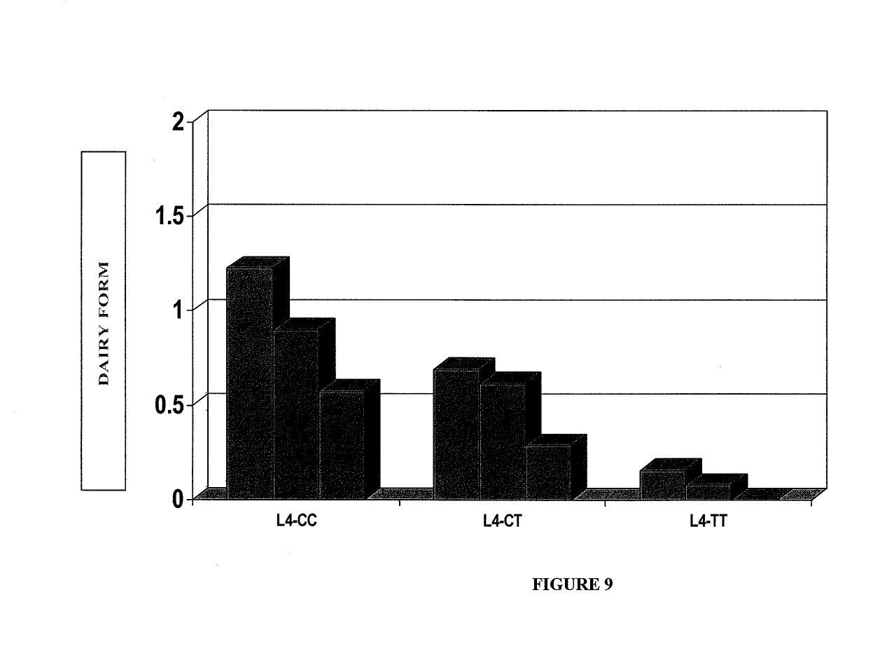

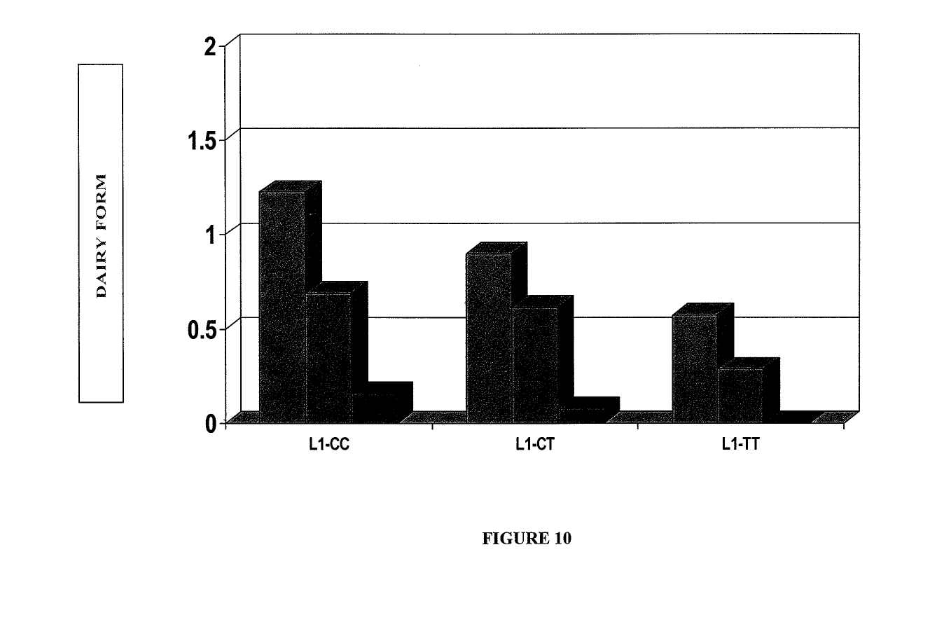

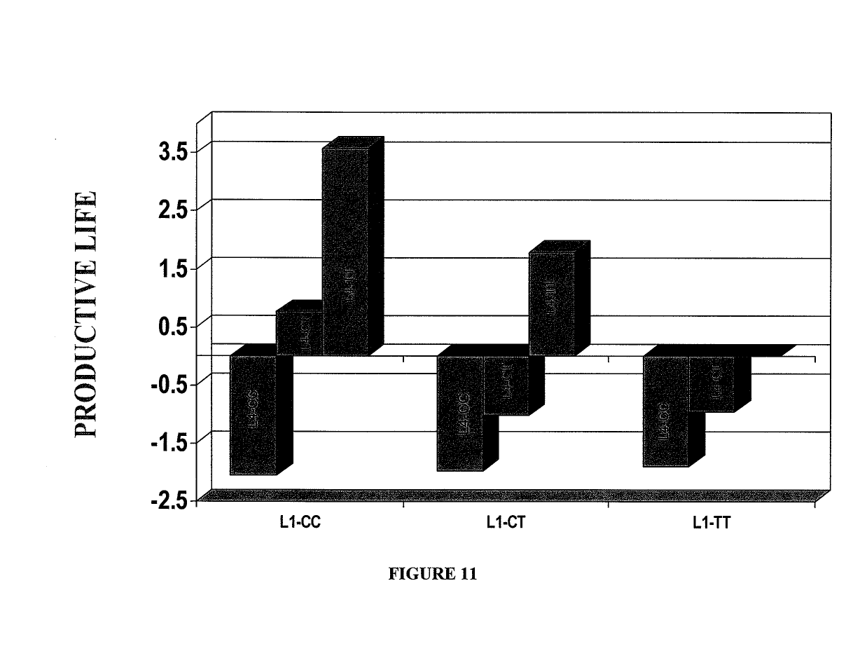

The SNP termed EXON2-FB described herein was identified previously by Buchanan et al. (2002), and constitutes a cytosine (C) to thymine (T) missense mutation at position 1759 in exon 2 of the coding region of the “wild type” bovine leptin gene. FIG. 4 illustrates the nucleotide sequence the exon 2 of the “wild type” bovine ob gene (SEQ ID NO. 4). This “wild type” exon 2 sequence has GenBank accession number AY138588. FIG. 5 illustrates the nucleotide sequence the EXON2-FB single nucleotide polymorphism of the bovine ob gene (SEQ ID NO. 5). This polymophic sequence differs from that of the “wild type” bovine ob gene sequence (SEQ ID NO. 1) in that nucleotide position 305 has a cytosine to thymine substitution. The nucleotide numbering system used herein for the identification of the EXON2-FB SNP is that used for the “wild type” bovine leptin exon 2 sequence SEQ ID NO. 4.

Productive life is time in the milking herd before removal by voluntary culling, involuntary culling, or death. Credits for each month in milk are obtained from standard lactation curves and are then summed across all lactations. Diminishing credits within lactation give cows more credit for beginning a new lactation than for continuing to milk in the previous lactation. Cows get 8 months credit for 305-d first lactation records, 10 months credit for second lactations, 10.2 months credit for third and later lactations, partial credits for shorter records, and extra credits for longer records. See, e.g., VanRaden, 2001. J. Dairy Sci. et al. 1993. J. Dairy Sci. 76:2758 and VanRaden et al. 2007. J. Dairy Sci. 90:2434.

The present invention also relates to the identification of genetic markers (single nucleotide polymorphisms (SNPs)) within a bovine gene encoding leptin, corticotropin-releasing hormone (CRH) and/or mitochondrial transcription factor A (“TFAM”) and their associations with economically relevant traits in beef cattle production, advantageously predicted transmitting abilities (PTA's) for productive life (PL).

The leptin genetic polymorphism of interest may be A1457G (see, e.g., Liefers et al., Anim Genet. 2005 April; 36(2):111-8 and Taniguchi et al., IUBMB Life. 2002 February; 53(2):131-5). Advantageously, the sequence of the leptin A1457G marker is TTCATTGTAGACACTTCTTTAAAAGAAACATTTCTTTATTTGACAGTTCCAGGCCT TAGTTTCAGCAGGCAGGATGTTTAGTCGCAGCATGAGAACTCTTA[A/G]CTGCGG CATGCGGGACCCAGTTCAGTTCCCTGACCAGATATCGAACCTGGGGCCCCTGCAT TTGGAAGCAGGGAGTCTTAGCCACTGGACCACCA (SEQ ID NO: 19) wherein the SNP is bracketed.

The CRH genetic polymorphism(s) of interest may be selected from the group consisting of AAFC03076794.1:g.9657C>T, c. 10718G>C, c. 10841G>A, c. 10893A>C and c.10936G>C. See, e.g., U.S. patent application Ser. No. 11,688,988 filed Mar. 21, 2007, the disclosure of which is incorporated by reference. Advantageously, the sequence of the CRH marker is CCCTTCCATTTTAGGGCTCGTTGACGTCATCAAGGAGGCGATAAATATCTGTTGA TATAATTGGATGTGAGATTCAGTGTTGAGATAGCAAAAATTCTGCCCCTCGTTCC CGGGCAGGGCCCTATGATTTATGCAGGAGCAGAGGCAGCG[C/T]GCAATCCAGCT GTCAAGAGAGCGTCAGCTTATTAGGCAAATGCTGCGTGGTTTCTGAAGAGGGTC GACACTATAAAATCCCCTTCCAGGCTCTGGTGTGGAGAAACTCAGAGCCCACGTC CGTGGAGAGACAGAAGAGGAAGAGAAGAGG (SEQ ID NO: 20) wherein the SNP is bracketed.

The TFAM genetic nucleotide polymorphism(s) of interest may be selected from the group consisting of an A to C substitution at the −1220 nucleotide position in the promoter of the TFAM gene, a T to C substitution at position −1212 in the promoter of the TFAM gene and a T to C substitution at position −995 in the promoter of the TFAM gene. See, e.g., U.S. patent application Ser. Nos. 11/441,928 and 11/441,935 filed May 26, 2006, the disclosures of which are incorporated by reference. Advantageously, the sequence of the TFAM markers are AAGAAAATTCTTACTAGTGTCATATTGAGCTGAGAGTCTTTCACTGGCCCGTTGA GTGCGACCACCAAGCCCACTTCTAAGTGGTAAGCAACTATCACCACTTCCTTTTC AAAAATTCTCAGTATTGAAAGGGACCTTTTAGATCATCTG[G/T]CCAAATCTCTCA CTCTGTCATGGAAGAAACAGGGTTGGATTTTGTTTGTTTGATTATTTTCTGAGTAC ACATTATGTACAAGGCCACAAACTTAGATAAAAAACACAGCCTTCTTTTCACCGC CCATGAAGTCTAGTGGC (SEQ ID NO: 21) and TGTTGGACGAGATCATTTCCCAACCAAAGCTCTGAGAACCTGTGACAATTATGCC TTGAGATATTGATCGATGTGCAGTACCGCCATTTTCCATGTGGACAATGGCATGA AAAAAGATTAGGATAACCTTATCTAAAGCTATCTAAGGAC [A/G] CTTCTAGCTCT CAGGTTCCATTTATTTCAAAGAGCCACTTCCTGAAACAGCTTTTTCTCTGTTGGAT AGGTTTGTAAGAAAATTCTTACTAGTGTCATATTGAGCTGAGAGTCTTTCACTGG CCCG (SEQ ID NO: 22) wherein the SNPs are bracketed.

The invention further relates to a method for sub-grouping animals according to genotype wherein the animals of each sub-group have a similar genotype in a leptin, CRH and/or TFAM gene which may comprise determining the genotype of each animal to be sub-grouped by determining the presence of one or more of the above SNPs, and segregating individual animals into sub-groups depending on whether the animals have, or do not have, the above SNPs in a leptin, CRH and/or TFAM gene.

To determine the genotype of a given animal according to the methods of the present invention, it is necessary to obtain a sample of genomic DNA from that animal. Typically, that sample of genomic DNA will be obtained from a sample of tissue or cells taken from that animal.

A tissue or cell sample may be taken from an animal at any time in the lifetime of an animal but before the carcass identity is lost. The tissue sample can comprise hair (including roots), hide, bone, buccal swabs, blood, saliva, milk, semen, embryos, muscle or any internal organs. In the method of the present invention, the source of the tissue sample, and thus also the source of the test nucleic acid sample, is not critical. For example, the test nucleic acid can be obtained from cells within a body fluid of the animal, or from cells constituting a body tissue of the animal. The particular body fluid from which cells are obtained is also not critical to the present invention. For example, the body fluid may be selected from the group consisting of blood, ascites, pleural fluid and spinal fluid. Furthermore, the particular body tissue from which cells are obtained is also not critical to the present invention. For example, the body tissue may be selected from the group consisting of skin, endometrial, uterine and cervical tissue. Both normal and tumor tissues can be used.

Typically, the tissue sample is marked with an identifying number or other indicia that relates the sample to the individual animal from which the sample was taken. The identity of the sample advantageously remains constant throughout the methods of the invention thereby guaranteeing the integrity and continuity of the sample during extraction and analysis. Alternatively, the indicia may be changed in a regular fashion that ensures that the data, and any other associated data, can be related back to the animal from which the data was obtained.

The amount/size of sample required is known to those skilled in the art. Ideally, the size/volume of the tissue sample retrieved should be as consistent as possible within the type of sample and the species of animal. For example, for cattle, non-limiting examples of sample sizes/methods include non-fatty meat: 0.0002 g to 0.0010 g; hide: 0.0004 g to 0.0010 g; hair roots: greater than five and less than twenty; buccal swabs: 15 to 20 seconds of rubbing with modest pressure in the area between outer lip and gum using one Cytosoft™ cytology brush; bone: 0.0020 g to 0.0040 g; and blood: 30 to 70 μL.

Generally, the tissue sample is placed in a container that is labeled using a numbering system bearing a code corresponding to the animal, for example, to the animal's ear tag. Accordingly, the genotype of a particular animal is easily traceable at all times.

In one embodiment of the invention, a sampling device and/or container may be supplied to the farmer, a slaughterhouse or retailer. The sampling device advantageously takes a consistent and reproducible sample from individual animals while simultaneously avoiding any cross-contamination of tissue. Accordingly, the size and volume of sample tissues derived from individual animals would be consistent.

According to the present invention, a sample of genomic DNA is obtained from the tissue sample of the livestock animal of interest. Whatever source of cells or tissue is used, a sufficient amount of cells must be obtained to provide a sufficient amount of DNA for analysis. This amount will be known or readily determinable by those skilled in the art.

DNA is isolated from the tissue/cells by techniques known to those skilled in the art (see, e.g., U.S. Pat. Nos. 6,548,256 and 5,989,431, Hirota et al., Jinrui Idengaku Zasshi. September 1989; 34(3):217-23 and John et al., Nucleic Acids Res. Jan. 25, 1991; 19(2):408; the disclosures of which are incorporated by reference in their entireties). For example, high molecular weight DNA may be purified from cells or tissue using proteinase K extraction and ethanol precipitation. DNA may be extracted from an animal specimen using any other suitable methods known in the art.

It is an object of the present invention to determine the genotype of a given animal of interest, in order to identify animals carrying specific alleles of the SNPs of the invention that are associated with circulating leptin levels, feed intake, growth rate, body weight, carcass merit and composition, and milk yield.

There are many methods known in the art for determining the genotype of an animal and for identifying whether a given DNA sample contains a particular SNP. Any method for determining genotype can be used for determining the ob genotype in the present invention. Such methods include, but are not limited to, amplimer sequencing, DNA sequencing, fluorescence spectroscopy, fluorescence resonance energy transfer (or “FRET”)-based hybridization analysis, high throughput screening, mass spectroscopy, nucleic acid hybridization, polymerase chain reaction (PCR), RFLP analysis and size chromatography (e.g., capillary or gel chromatography), all of which are well known to one of skill in the art. In particular, methods for determining nucleotide polymorphisms, particularly single nucleotide polymorphisms, are described in U.S. Pat. Nos. 6,514,700; 6,503,710; 6,468,742; 6,448,407; 6,410,231; 6,383,756; 6,358,679; 6,322,980; 6,316,230; and 6,287,766 and reviewed by Chen and Sullivan, Pharmacogenomics J 2003; 3(2):77-96, the disclosures of which are incorporated by reference in their entireties.

In one embodiment, the presence or absence of the SNPs of the present invention is determined by sequencing the region of the genomic DNA sample that spans the polymorphic locus. Many methods of sequencing genomic DNA are known in the art, and any such method can be used, see for example Sambrook et al., Molecular Cloning; A Laboratory Manual 2d ed. (1989). For example, as described below, a DNA fragment spanning the location of the SNP of interest can amplified using the polymerase chain reaction or some other cyclic polymerase mediated amplification reaction. The amplified region of DNA can then be sequenced using any method known in the art. Advantageously, the nucleic acid sequencing is by automated methods (reviewed by Meldrum, Genome Res. September 2000; 10(9):1288-303, the disclosure of which is incorporated by reference in its entirety), for example using a Beckman CEQ 8000 Genetic Analysis System (Beckman Coulter Instruments, Inc.). Methods for sequencing nucleic acids include, but are not limited to, automated fluorescent DNA sequencing (see, e.g., Watts & MacBeath, Methods Mol Biol. 2001; 167:153-70 and MacBeath et al., Methods Mol Biol. 2001; 167:119-52), capillary electrophoresis (see, e.g., Bosserhoff et al., Comb Chem High Throughput Screen. December 2000; 3(6):455-66), DNA sequencing chips (see, e.g., Jain, Pharmacogenomics. August 2000; 1(3):289-307), mass spectrometry (see, e.g., Yates, Trends Genet. January 2000; 16(1):5-8), pyrosequencing (see, e.g., Ronaghi, Genome Res. January 2001; 11(1):3-11), and ultrathin-layer gel electrophoresis (see, e.g., Guttman & Ronai, Electrophoresis. December 2000; 21 (18):3952-64), the disclosures of which are hereby incorporated by reference in their entireties. The sequencing can also be done by any commercial company. Examples of such companies include, but are not limited to, the University of Georgia Molecular Genetics Instrumentation Facility (Athens, Ga.) or SeqWright DNA Technologies Services (Houston, Tex.).

### vi) Determining the Genotype Using Cyclic Polymerase Mediated Amplification

In certain embodiments of the present invention, the detection of a given SNP can be performed using cyclic polymerase-mediated amplification methods. Any one of the methods known in the art for amplification of DNA may be used, such as for example, the polymerase chain reaction (PCR), the ligase chain reaction (LCR) (Barany, F., Proc. Natl. Acad. Sci. (U.S.A.) 88:189-193 (1991)), the strand displacement assay (SDA), or the oligonucleotide ligation assay (“OLA”) (Landegren, U. et al., Science 241:1077-1080 (1988)). Nickerson, D. A. et al. have described a nucleic acid detection assay that combines attributes of PCR and OLA (Nickerson, D. A. et al., Proc. Natl. Acad. Sci. (U.S.A.) 87:8923-8927 (1990)). Other known nucleic acid amplification procedures, such as transcription-based amplification systems (Malek, L. T. et al., U.S. Pat. No. 5,130,238; Davey, C. et al., European Patent Application 329,822; Schuster et al., U.S. Pat. No. 5,169,766; Miller, H. I. et al., PCT Application WO89/06700; Kwoh, D. et al., Proc. Natl. Acad. Sci. (U.S.A.) 86:1173 (1989); Gingeras, T. R. et al., PCT Application WO88/10315)), or isothermal amplification methods (Walker, G. T. et al., Proc. Natl. Acad. Sci. (U.S.A.) 89:392-396 (1992)) may also be used.

The most advantageous method of amplifying DNA fragments containing the SNPs of the invention employs PCR (see e.g., U.S. Pat. Nos. 4,965,188; 5,066,584; 5,338,671; 5,348,853; 5,364,790; 5,374,553; 5,403,707; 5,405,774; 5,418,149; 5,451,512; 5,470,724; 5,487,993; 5,523,225; 5,527,510; 5,567,583; 5,567,809; 5,587,287; 5,597,910; 5,602,011; 5,622,820; 5,658,764; 5,674,679; 5,674,738; 5,681,741; 5,702,901; 5,710,381; 5,733,751; 5,741,640; 5,741,676; 5,753,467; 5,756,285; 5,776,686; 5,811,295; 5,817,797; 5,827,657; 5,869,249; 5,935,522; 6,001,645; 6,015,534; 6,015,666; 6,033,854; 6,043,028; 6,077,664; 6,090,553; 6,168,918; 6,174,668; 6,174,670; 6,200,747; 6,225,093; 6,232,079; 6,261,431; 6,287,769; 6,306,593; 6,440,668; 6,468,743; 6,485,909; 6,511,805; 6,544,782; 6,566,067; 6,569,627; 6,613,560; 6,613,560 and 6,632,645; the disclosures of which are incorporated by reference in their entireties), using primer pairs that are capable of hybridizing to the proximal sequences that define or flank a polymorphic site in its double-stranded form.

To perform a cyclic polymerase mediated amplification reaction according to the present invention, the primers are hybridized or annealed to opposite strands of the target DNA, the temperature is then raised to permit the thermostable DNA polymerase to extend the primers and thus replicate the specific segment of DNA spanning the region between the two primers. Then the reaction is thermocycled so that at each cycle the amount of DNA representing the sequences between the two primers is doubled, and specific amplification of the ob gene DNA sequences, if present, results.

Any of a variety of polymerases can be used in the present invention. For thermocyclic reactions, the polymerases are thermostable polymerases such as Taq, KlenTaq, Stoffel Fragment, Deep Vent, Tth, Pfu, Vent, and UlTma, each of which are readily available from commercial sources. For non-thermocyclic reactions, and in certain thermocyclic reactions, the polymerase will often be one of many polymerases commonly used in the field, and commercially available, such as DNA pol 1, Klenow fragment, T7 DNA polymerase, and T4 DNA polymerase. Guidance for the use of such polymerases can readily be found in product literature and in general molecular biology guides.

Typically, the annealing of the primers to the target DNA sequence is carried out for about 2 minutes at about 37-55 C, extension of the primer sequence by the polymerase enzyme (such as Taq polymerase) in the presence of nucleoside triphosphates is carried out for about 3 minutes at about 70-75 C, and the denaturing step to release the extended primer is carried out for about 1 minute at about 90-95 C. However, these parameters can be varied, and one of skill in the art would readily know how to adjust the temperature and time parameters of the reaction to achieve the desired results. For example, cycles may be as short as 10, 8, 6, 5, 4.5, 4, 2, 1, 0.5 minutes or less.

Also, “two temperature” techniques can be used where the annealing and extension steps may both be carried out at the same temperature, typically between about 60-65 C, thus reducing the length of each amplification cycle and resulting in a shorter assay time.

Typically, the reactions described herein are repeated until a detectable amount of product is generated. Often, such detectable amounts of product are between about 10 ng and about 100 ng, although larger quantities, e.g. 200 ng, 500 ng, 1 mg or more can also, of course, be detected. In terms of concentration, the amount of detectable product can be from about 0.01 pmol, 0.1 pmol, 1 pmol, 10 pmol, or more. Thus, the number of cycles of the reaction that are performed can be varied, the more cycles are performed, the more amplified product is produced. In certain embodiments, the reaction comprises 2, 5, 10, 15, 20, 30, 40, 50, or more cycles.

For example, the PCR reaction may be carried out using about 25-50 mu.l samples containing about 0.01 to 1.0 ng of template amplification sequence, about 10 to 100 pmol of each generic primer, about 1.5 units of Taq DNA polymerase (Promega Corp.), about 0.2 mM dDATP, about 0.2 mM dCTP, about 0.2 mM dGTP, about 0.2 mM dTTP, about 15 mM MgCl.sub.2, about 10 mM Tris-HCl (pH 9.0), about 50 mM KCl, about 1 .mu.g/ml gelatin, and about 10 mu.l/ml Triton X-100 (Saiki, 1988).

Those of skill in the art are aware of the variety of nucleotides available for use in the cyclic polymerase mediated reactions. Typically, the nucleotides will consist at least in part of deoxynucleotide triphosphates (dNTPs), which are readily commercially available. Parameters for optimal use of dNTPs are also known to those of skill, and are described in the literature. In addition, a large number of nucleotide derivatives are known to those of skill and can be used in the present reaction. Such derivatives include fluorescently labeled nucleotides, allowing the detection of the product including such labeled nucleotides, as described below. Also included in this group are nucleotides that allow the sequencing of nucleic acids including such nucleotides, such as chain-terminating nucleotides, dideoxynucleotides and boronated nuclease-resistant nucleotides. Commercial kits containing the reagents most typically used for these methods of DNA sequencing are available and widely used. Other nucleotide analogs include nucleotides with bromo-, iodo-, or other modifying groups, which affect numerous properties of resulting nucleic acids including their antigenicity, their replicatability, their melting temperatures, their binding properties, etc. In addition, certain nucleotides include reactive side groups, such as sulfhydryl groups, amino groups, N-hydroxysuccinimidyl groups, that allow the further modification of nucleic acids comprising them.

The present invention provides oligonucleotides that can be used as primers to amplify specific nucleic acid sequences of the ob gene in cyclic polymerase-mediated amplification reactions, such as PCR reactions. These primers are useful in detecting the UASMS1 or UASMS2 SNPs in the leptin promoter, and the exon2-FB SNP in exon 2 of the leptin gene. In certain embodiments, these primers consist of oligonucleotide fragments. Such fragments should be of sufficient length to enable specific annealing or hybridization to the nucleic acid sample. The sequences typically will be about 8 to about 44 nucleotides in length, but may be longer. Longer sequences, e.g., from about 14 to about 50, are advantageous for certain embodiments.

In embodiments where it is desired to amplify a fragment of DNA comprising the UASMS1 or UASMS2 SNPs, primers having contiguous stretches of about 8, 9, 10, 11, 12, 13, 14, 15, 16, 17, 18, 19, 20, 21, 22, 23, or 24 nucleotides from SEQ ID NO: 1 (the leptin promoter sequence) are contemplated. In embodiments where it is desired to amplify a fragment of DNA comprising the EXON2-FB SNP, primers having contiguous stretches of about 8, 9, 10, 11, 12, 13, 14, 15, 16, 17, 18, 19, 20, 21, 22, 23, or 24 nucleotides from SEQ ID NO: 4 (exon 2 of the leptin gene) are contemplated.

Although various different lengths of primers can be used, and the exact location of the stretch of contiguous nucleotides in leptin gene used to make the primer can vary, it is important that the sequences to which the forward and reverse primers anneal are located on either side of the particular nucleotide position that is substituted in the SNP to be amplified. For example, when designing primers for amplification of the UASMS1 polymorphism, one primer must be located upstream of (not overlapping with) nucleotide position 207 of the leptin promoter (SEQ ID NO: 1, or 2), and the other primer must be located downstream of (not overlapping with) nucleotide position 207 of the leptin promoter (SEQ ID NO: 1, or 2). When designing primers for amplification of the UASMS2 polymorphism, one primer must be located upstream of (not overlapping with) nucleotide position 528 of the leptin promoter (SEQ ID NO: 1, or 3), and the other primer must be located downstream of (not overlapping with) nucleotide position 528 of the leptin promoter (SEQ ID NO: 1, or 3). Finally, when designing primers for amplification of the EXON2-FB polymorphism one primer must be located upstream of (not overlapping with) nucleotide position 304 of exon 2 (SEQ ID NO: 4), and the other primer must be located downstream of (not overlapping with) nucleotide position 304 of exon 2.

In a preferred embodiment, a fragment of DNA spanning and containing the location of the UASMS1 polymorphism is amplified from a nucleic acid sample using a forward primer having the sequence 5′-GGCACAATCCTGTGTATTGGTAAGA-3′ (SEQ ID NO: 7), and a reverse primer having the sequence 5′-GTCCATGTACCATTGCCCAATTT-3′ (SEQ ID NO: 8).

Similarly, in a preferred embodiment, a fragment of DNA spanning the location of the UASMS2 polymorphism is amplified from a nucleic acid sample using a forward primer having the sequence 5′-AGGTGCCCAGGGACTCA-3′- (SEQ ID NO: 9), and a reverse primer having the sequence 5′-CAACAAAGGCCGTGTGACA-3′ (SEQ ID NO: 10).

Likewise, for amplification of a fragment of DNA spanning the location of the EXON2-FB polymorphism, it is preferred that a forward primer having the sequence 5′-GGCTTTGGCCCTATCTGTCTTAC-3′ (SEQ ID NO: 11), and a reverse primer having the sequence 5′-CTTGATGAGGGTTTTGGTGTCA-3′ (SEQ ID NO: 12), is used.

The above methods employ primers located on either side of, and not overlapping with, the SNP in order to amplify a fragment of DNA that includes the nucleotide position at which the SNP is located. Such methods require additional steps, such as sequencing of the fragment, or hybridization of allele specific probes to the fragment, in order to determine the genotype at the polymorphic site. However, in some embodiments of the present invention, the amplification method is itself a method for determining the genotype of the polymorphic site, as for example, in “allele-specific PCR”. In allele-specific PCR, primer pairs are chosen such that amplification itself is dependent upon the input template nucleic acid containing the polymorphism of interest. In such embodiments, primer pairs are chosen such that at least one primer spans the actual nucleotide position of the SNP and is therefore an allele-specific oligonucleotide primer. Typically, the primers contain a single allele-specific nucleotide at the 3′ terminus preceded by bases that are complementary to the gene of interest. The PCR reaction conditions are adjusted such that amplification by a DNA polymerase proceeds from matched 3′-primer termini, but does not proceed where a mismatch occurs. Allele specific PCR can be performed in the presence of two different allele-specific primers, one specific for each allele, where each primer is labeled with a different dye, for example one allele specific primer may be labeled with a green dye (e.g. fluorescein) and the other allele specific primer labeled with a red dye (e.g. sulforhodamine). Following amplification, the products are analyzed for green and red fluorescence. The aim is for one homozygous genotype to yield green fluorescence only, the other homozygous genotype to give red fluorescence only, and the heterozygous genotype to give mixed red and green fluorescence.

Thus, to perform allele specific PCR to detect the UASMS1 polymorphism, one primer must overlap nucleotide position 207 of SEQ ID NO: 1 or SEQ ID NO: 2 such that nucleotide position 207 is at the 3′ terminus of the primer. Similarly, to perform allele specific PCR to detect the UASMS2 polymorphism, one primer must overlap nucleotide position 528 of SEQ ID NO: 1 or SEQ ID NO: 3 such that nucleotide position 528 is at the 3′ terminus of the primer. Finally, when designing allele specific primers for detection of the EXON2-FB polymorphism, one primer must overlap nucleotide position 304 of SEQ ID NO: 4 or SEQ ID NO: 5 such that nucleotide position 304 is at the 3′ terminus of the primer.

Methods for performing allele specific PCR are well known in the art, and any such methods may be used. For example suitable methods are taught in Myakishev et al. Genome Research, vol 1, p 163-169 (2001), Alexander et al. Mol Biotechnol. vol 28(3), p 171-174 (2004), and Ruano et al. Nucleic Acids Res. vol 17(20), p 8392 (1989), the contents of which are incorporated by reference. In some embodiments of the present invention, allele-specific primers are chosen so that amplification creates a restriction site, facilitating identification of a polymorphic site. To perform, allele specific PCR the reaction conditions must be carefully adjusted such that the allele specific primer will only bind to one allele and not the alternative allele, for example, in some embodiments the conditions are adjusted so that the primers will only bind where there is a 100% match between the primer sequence and the DNA, and will not bind if there is a single nucleotide mismatch.

In certain embodiments of the present invention, the detection of a given SNP can be performed using oligonucleotide probes that bind or hybridize to the DNA. The present invention provides oligonucleotide probes to detect the UASMS1, UASMS2 or SNPs in the bovine leptin promoter, or the EXON2-FB SNP in exon 2 of the bovine leptin gene.

In certain embodiments, these probes consist of oligonucleotide fragments. Such fragments should be of sufficient length to provide specific hybridization to the nucleic acid sample. The sequences typically will be about 8 to about 50 nucleotides, but may be longer. Nucleic acid probes having contiguous stretches of about 8, 9, 10, 11, 12, 13, 14, 15, 16, 17, 18, 19, 20, 21, 22, 23, or 24 nucleotides from a sequence selected from SEQ ID NO: 1 (wild-type bovine leptin promoter), SEQ ID NO:2 (bovine leptin promoter with UASMS1 polymorphism), SEQ ID NO:3 (bovine leptin promoter with UASMS2 polymorphism), SEQ ID NO:5 (wild-type bovine leptin exon 2) or SEQ ID NO:6 (leptin exon 2 with EXON2-FB polymorphism) are contemplated.

Although various different lengths of probes can be used, and the precise location of the stretch of contiguous nucleotides in the leptin gene from which the probe sequence is derived can vary, the probe sequence must span the particular nucleotide position that is substituted in the particular SNP to be detected. For example, probes designed for detection of the bovine UASMS1 polymorphism must span nucleotide position 207 of the bovine leptin promoter (SEQ ID NO: 2). Probes designed for detection of the bovine UASMS2 polymorphism must span nucleotide position 528 of the bovine leptin promoter (SEQ ID NO: 3). Finally, probes designed for detection of the bovine exon2-FB polymorphism must span nucleotide position 304 of exon 2 of the bovine leptin gene (SEQ ID NO: 4).

These probes will be useful in a variety of hybridization embodiments, such as Southern blotting, Northern blotting, and hybridization disruption analysis. Also the probes of the invention can be used to detect SNPs in amplified sequences, such as amplified PCR products generated using the primers described above. For example, in one embodiment a target nucleic acid is first amplified, such as by PCR or strand displacement amplification (SDA), and the amplified double stranded DNA product is then denatured and hybridized with a probe.

In other embodiments double stranded DNA (amplified or not) is denatured and hybridized with a probe of the present invention and then the hybridization complex is subjected to destabilizing or disrupting conditions. By determining the level of disruption energy required wherein the probe has different disruption energy for one allele as compared to another allele, the genotype of a gene at a polymorphic locus can be determined. In one example, there can be lower disruption energy, e.g., melting temperature, for an allele that harbors a cytosine residue at a polymorphic locus, and a higher required energy for an allele with a thymine residue at that polymorphic locus. This can be achieved where the probe has 100% homology with one allele (a perfectly matched probe), but has a single mismatch with the alternative allele. Since the perfectly matched probe is bound more tightly to the target DNA than the mismatched probe, it requires more energy to cause the hybridized probe to dissociate.

In one embodiment the destabilizing conditions comprise an elevation of temperature. The higher the temperature, the greater the degree of destabilization. In another embodiment, the destabilizing conditions comprise subjecting the hybridization complex to a temperature gradient, whereby, as the temperature is increased, the degree of destabilization increases. In an alternative embodiment, the destabilizing conditions comprise treatment with a destabilizing compound, or a gradient comprising increasing amounts of such a compound. Suitable destabilizing compounds include, but are not limited to, salts and urea. Methods of destabilizing or denaturing hybridization complexes are well known in the art, and any such method may be used in accordance with the present invention. For example, methods of destabilizing or denaturing hybridization complexes are taught by Sambrook et al., Molecular Cloning; A Laboratory Manual 2d ed. (1989).

For optimal detection of single-base pair mismatches, it is preferable that there is about a 1° C. to about a 10° C. difference in melting temperature of the probe DNA complex when bound to one allele as opposed to the alternative allele at the polymorphic site. Thus, when the temperature is raised above the melting temperature of a probe:DNA duplex corresponding to one of the alleles, that probe will disassociate.

In one embodiment of the above method, a second (“anchor”) probe can be used. Generally, the anchor probe is not specific to either allele, but hybridizes regardless of what nucleotide is present at the polymorphic locus. The anchor probe does not affect the disruption energy required to disassociate the hybridization complex but, instead, contains a complementary label for using with the first (“sensor”) probe, for example for use in fluorescence resonance energy transfer or “FRET.” A sensor probe acquires energy from the anchor probe once conditions are adequate for hybridization between the target DNA and the anchor and sensor probes. Once hybridization occurs, the anchor probe transfers its florescence energy to the sensor probe, which only will emit a specific wavelength after it has acquired the energy from the anchor probe. Detection of the SNP occurs as the temperature is raised at a predetermined rate, and a reading is acquired from the florescent light emitted. If there is a single base mismatch of the probe and target DNA caused by the presence of the alternative polymorphic nucleotide (i.e. the SNP) the sensor probe will dissociate sooner, or at a lower temperature, since the homology between the genomic DNA and the sensor probe will be less than that of genomic DNA that does not harbor the altered nucleotide or SNP. Thus, there will be a loss of fluorescence that can be detected. Where the probe is designed to bind to the wild-type sequence, the dissociation of the probe from the DNA (i.e. the “melting”) will occur at a lower temperature if the SNP is present, since the stability of the binding of the probe to the SNP is slightly less than for the wild-type sequence. This occurs, obviously, on both chromosomes at the same time, thus yielding either a reading of two identical melting temperatures for a homozygote, or a reading of two different melting temperatures for the heterozygote. For example, where a probe is designed to have the sequence of the C-containing allele of the UASMS1 polymorphism, the probe will dissociate or melt at a lower temperature in DNA samples from individuals that harbor two copies of the polymorphic T-containing allele, than in individuals that harbor two copies of the C-containing allele.

In other embodiments, two different “allele-specific probes” can be used for analysis of a SNP, a first allele-specific probe for detection of one allele, and a second allele-specific probe for the detection of the alternative allele. For example, in one embodiment the different alleles of the UASMS1 ob polymorphism can be detected using two different allele-specific probes, one for detecting the T-containing allele at nucleotide position 207 of the ob gene promoter, and another for detecting the C-containing allele at nucleotide position 207 of the ob gene promoter. In a preferred embodiment an oligonucleotide probe having the sequence of 5′-CTTTCACCTAGTATATCTAG-3′ (SEQ ID NO: 13) is used to detect the T-containing allele, and an oligonucleotide probe having the sequence of 5′-TCTTTCACCTAGTATGTCTAG-3′ (SEQ ID NO: 14) is used to detect the C-containing allele.

In another embodiment the different alleles of the UASMS2 ob polymorphism can be detected using two different allele-specific probes, one for detecting the T-containing allele at nucleotide position 528 of the ob gene promoter, and another for detecting the C-containing allele at nucleotide position 528 of the ob gene promoter. In a preferred embodiment an oligonucleotide probe having the sequence of 5′-AAGCTCTAGAGCCTATGT-3′ (SEQ ID NO: 15) is used to detect the T-containing allele, and an oligonucleotide probe having the sequence of 5′-CAAGCTCTAGAGCCTGTGT-3′ (SEQ ID NO: 16) is used to detect the C-containing allele.

In a further embodiment the different alleles of the EXON2-FB ob polymorphism can be detected using two different allele-specific probes, one for detecting the T-containing allele at nucleotide position 305 of exon 2 of the ob gene, and another for detecting the C-containing allele at nucleotide position 305 of exon 2 of the ob gene. In a preferred embodiment an oligonucleotide probe having the sequence of 5′-CCTTGCAGATGGG-3′ (SEQ ID NO: 17) is used to detect the T-containing allele, and an oligonucleotide probe having the sequence of 5′-CCTTGCGGATGGG-3′ (SEQ ID NO: 18) is used to detect the C-containing allele.

Whichever probe sequences and hybridization methods are used, one skilled in the art can readily determine suitable hybridization conditions, such as temperature and chemical conditions. Such hybridization methods are well known in the art. For example, for applications requiring high selectivity, one will typically desire to employ relatively stringent conditions for the hybridization reactions, e.g., one will select relatively low salt and/or high temperature conditions, such as provided by about 0.02 M to about 0.10 M NaCl at temperatures of about 50 C to about 70 C. Such high stringency conditions tolerate little, if any, mismatch between the probe and the template or target strand, and are particularly suitable for detecting specific SNPs according to the present invention. It is generally appreciated that conditions can be rendered more stringent by the addition of increasing amounts of formamide. Other variations in hybridization reaction conditions are well known in the art (see for example, Sambrook et al., Molecular Cloning; A Laboratory Manual 2d ed. (1989)).

In addition to the SNPs described above, it will be appreciated by those skilled in the art that other DNA sequence polymorphisms of the ob gene may exist within a population. Such natural allelic variations can typically result in about 1-5% variance in the nucleotide sequence of the gene. For example, SEQ ID NO 2 provides a sequence of a region of the ob gene promoter containing a polymorphism at nucleotide position 207 (i.e. the UASMS1 SNP). It is possible that other polymorphic loci may also exist within this fragment. In addition to naturally-occurring allelic variants of the nucleotide sequence, the skilled artisan will further appreciate that changes can be introduced by mutation into the nucleotide sequence of the nucleotide sequences described herein. Any and all such additional nucleotide variations are intended to be within the scope of the invention. Thus, for example a probe according to the present invention may be designed to bind to a sequence of the ob gene containing not only the UASMS1 polymorphism, but also other SNPs that may occur within the same region.

Moreover, nucleic acid molecules that differ from the sequences of the primers and probes disclosed herein, are intended to be within the scope of the invention. Nucleic acid sequences that are complementary to these sequences, or that are hybridizable to the sequences described herein under conditions of standard or stringent hybridization, and also analogs and derivatives are also intended to be within the scope of the invention. Advantageously, such variations will differ from the sequences described herein by only a small number of nucleotides, for example by 1, 2, or 3 nucleotides.

Nucleic acid molecules corresponding to natural allelic variants, homologues (i.e., nucleic acids derived from other species), or other related sequences (e.g., paralogs) of the sequences described herein can be isolated based on their homology to the nucleic acids disclosed herein, for example by performing standard or stringent hybridization reactions using all or a portion of the sequences of the invention as probes. Such methods for nucleic acid hybridization and cloning are well known in the art.

Similarly, a nucleic acid molecule of the invention may include only a fragment of the specific sequences described. Fragments provided herein are defined as sequences of at least 6 (contiguous) nucleic acids, a length sufficient to allow for specific hybridization of nucleic acid primers or probes, and are at most some portion less than a full-length sequence. Fragments may be derived from any contiguous portion of a nucleic acid sequence of choice. Derivatives and analogs may be full length or other than full length, if the derivative or analog contains a modified nucleic acid or amino acid, as described below.

Derivatives, analogs, homologues, and variants of the nucleic acids of the invention include, but are not limited to, molecules comprising regions that are substantially homologous to the nucleic acids of the invention, in various embodiments, by at least about 70%, 80%, 85%, 90%, 95%, 96%, 97%, 98%, or even 99% identity (with an advantageous identity of 80-99%) over a nucleic acid sequence of identical size or when compared to an aligned sequence in which the alignment is done by a computer homology program known in the art.

The primers and probes described herein may be readily prepared by, for example, directly synthesizing the fragment by chemical means or by introducing selected sequences into recombinant vectors for recombinant production. Methods for making a vector or recombinants or plasmid for amplification of the fragment either in vivo or in vitro can be any desired method, e.g., a method which is by or analogous to the methods disclosed in, or disclosed in documents cited in: U.S. Pat. Nos. 4,603,112; 4,769,330; 4,394,448; 4,722,848; 4,745,051; 4,769,331; 4,945,050; 5,494,807; 5,514,375; 5,744,140; 5,744,141; 5,756,103; 5,762,938; 5,766,599; 5,990,091; 5,174,993; 5,505,941; 5,338,683; 5,494,807; 5,591,639; 5,589,466; 5,677,178; 5,591,439; 5,552,143; 5,580,859; 6,130,066; 6,004,777; 6,130,066; 6,497,883; 6,464,984; 6,451,770; 6,391,314; 6,387,376; 6,376,473; 6,368,603; 6,348,196; 6,306,400; 6,228,846; 6,221,362; 6,217,883; 6,207,166; 6,207,165; 6,159,477; 6,153,199; 6,090,393; 6,074,649; 6,045,803; 6,033,670; 6,485,729; 6,103,526; 6,224,882; 6,312,682; 6,348,450 and 6,312,683; U.S. patent application Ser. No. 920,197, filed Oct. 16, 1986; WO 90/01543; WO91/11525; WO 94/16716; WO 96/39491; WO 98/33510; EP 265785; EP 0 370 573; Andreansky et al., Proc. Natl. Acad. Sci. USA 1996; 93:11313-11318; Ballay et al., EMBO J. 1993; 4:3861-65; Felgner et al., J. Biol. Chem. 1994; 269:2550-2561; Frolov et al., Proc. Natl. Acad. Sci. USA 1996; 93:11371-11377; Graham, Tibtech 1990; 8:85-87; Grunhaus et al., Sem. Virol. 1992; 3:237-52; Ju et al., Diabetologia 1998; 41:736-739; Kitson et al., J. Virol. 1991; 65:3068-3075; McClements et al., Proc. Natl. Acad. Sci. USA 1996; 93:11414-11420; Moss, Proc. Natl. Acad. Sci. USA 1996; 93:11341-11348; Paoletti, Proc. Natl. Acad. Sci. USA 1996; 93:11349-11353; Pennock et al., Mol. Cell. Biol. 1984; 4:399-406; Richardson (Ed), Methods in Molecular Biology 1995; 39, “Baculovirus Expression Protocols,” Humana Press Inc.; Smith et al. (1983) Mol. Cell. Biol. 1983; 3:2156-2165; Robertson et al., Proc. Natl. Acad. Sci. USA 1996; 93:11334-11340; Robinson et al., Sem. Immunol. 1997; 9:271; and Roizman, Proc. Natl. Acad. Sci. USA 1996; 93:11307-11312.

Oligonucleotide sequences used as primers or probes according to the present invention may be labeled with a detectable moiety. As used herein the term “sensors” refers to such primers or probes labeled with a detectable moiety. Various labeling moieties are known in the art. Said moiety may be, for example, a radiolabel (e.g., .sup.3H, .sup.125I, sup.35S, .sup. 14C, .sup.32P, etc.), detectable enzyme (e.g. horse radish peroxidase (HRP), alkaline phosphatase etc.), a fluorescent dye (e.g., fluorescein isothiocyanate, Texas red, rhodamine, Cy3, Cy5, Bodipy, Bodipy Far Red, Lucifer Yellow, Bodipy 630/650-X, Bodipy R6G-X and 5-CR 6G, and the like), a calorimetric label such as colloidal gold or colored glass or plastic (e.g. polystyrene, polypropylene, latex, etc.), beads, or any other moiety capable of generating a detectable signal such as a calorimetric, fluorescent, chemiluminescent or electrochemiluminescent (ECL) signal.

Primers or probes may be labeled directly or indirectly with a detectable moiety, or synthesized to incorporate the detectable moiety. In one embodiment, a detectable label is incorporated into a nucleic acid during at least one cycle of a cyclic polymerase-mediated amplification reaction. For example, polymerases can be used to incorporate fluorescent nucleotides during the course of polymerase-mediated amplification reactions. Alternatively, fluorescent nucleotides may be incorporated during synthesis of nucleic acid primers or probes. To label an oligonucleotide with the fluorescent dye, one of conventionally-known labeling methods can be used (Nature Biotechnology, 14, 303-308, 1996; Applied and Environmental Microbiology, 63, 1143-1147, 1997; Nucleic Acids Research, 24, 4532-4535, 1996). An advantageous probe is one labeled with a fluorescent dye at the 3′ or 5′ end and containing G or C as the base at the labeled end. If the 5′ end is labeled and the 3′ end is not labeled, the OH group on the C atom at the 3′-position of the 3′ end ribose or deoxyribose may be modified with a phosphate group or the like although no limitation is imposed in this respect.

Spectroscopic, photochemical, biochemical, immunochemical, electrical, optical or chemical means can be used to detect such labels. The detection device and method may include, but is not limited to, optical imaging, electronic imaging, imaging with a CCD camera, integrated optical imaging, and mass spectrometry. Further, the amount of labeled or unlabeled probe bound to the target may be quantified. Such quantification may include statistical analysis. In other embodiments the detection may be via conductivity differences between concordant and discordant sites, by quenching, by fluorescence perturbation analysis, or by electron transport between donor and acceptor molecules.

In yet another embodiment, detection may be via energy transfer between molecules in the hybridization complexes in PCR or hybridization reactions, such as by fluorescence energy transfer (FET) or fluorescence resonance energy transfer (FRET). In FET and FRET methods, one or more nucleic acid probes are labeled with fluorescent molecules, one of which is able to act as an energy donor and the other of which is an energy acceptor molecule. These are sometimes known as a reporter molecule and a quencher molecule respectively. The donor molecule is excited with a specific wavelength of light for which it will normally exhibit a fluorescence emission wavelength. The acceptor molecule is also excited at this wavelength such that it can accept the emission energy of the donor molecule by a variety of distance-dependent energy transfer mechanisms. Generally the acceptor molecule accepts the emission energy of the donor molecule when they are in close proximity (e.g. on the same, or a neighboring molecule). FET and FRET techniques are well known in the art, and can be readily used to detect the SNPs of the present invention. See for example U.S. Pat. Nos. 5,668,648, 5,707,804, 5,728,528, 5,853,992, and 5,869,255 (for a description of FRET dyes), Tyagi et al. Nature Biotech. vol. 14, p 303-8 (1996), and Tyagi et al., Nature Biotech. vol 16, p 49-53 (1998) (for a description of molecular beacons for FET), and Mergny et al. Nucleic Acid Res. vol 22, p 920-928, (1994) and Wolf et al. PNAS vol 85, p 8790-94 (1988) (for general descriptions and methods fir FET and FRET), each of which is hereby incorporated by reference.

The oligonucleotide primers and probes of the present invention have commercial applications in diagnostic kits for the detection of the UASMS1, UASMS2, and EXON2-FB ob gene SNPs in livestock specimens. A test kit according to the invention may comprise any of the oligonucleotide primers or probes according to the invention. Such a test kit may additionally comprise one or more reagents for use in cyclic polymerase mediated amplification reactions, such as DNA polymerases, nucleotides (dNTPs), buffers, and the like. An SNP detection kit may also include, a lysing buffer for lysing cells contained in the specimen.

A test kit according to the invention may comprise a pair of oligonucleotide primers according to the invention and a probe comprising an oligonucleotide according to the invention. In some embodiments such a kit will contain two allele specific oligonucleotide probes. Advantageously, the kit further comprises additional means, such as reagents, for detecting or measuring the binding or the primers and probes of the present invention, and also ideally a positive and negative control.

The present invention further encompasses probes according to the present invention that are immobilized on a solid or flexible support, such as paper, nylon or other type of membrane, filter, chip, glass slide, microchips, microbeads, or any other such matrix, all of which are within the scope of this invention. The probe of this form is now called a “DNA chip”. These DNA chips can be used for analyzing the SNPs of the present invention. The present invention further encompasses arrays or microarrays of nucleic acid molecules that are based on one or more of the sequences described herein. As used herein “arrays” or “microarrays” refers to an array of distinct polynucleotides or oligonucleotides synthesized on a solid or flexible support, such as paper, nylon or other type of membrane, filter, chip, glass slide, or any other suitable solid support. In one embodiment, the microarray is prepared and used according to the methods and devices described in U.S. Pat. Nos. 5,446,603; 5,545,531; 5,807,522; 5,837,832; 5,874,219; 6,114,122; 6,238,910; 6,365,418; 6,410,229; 6,420,114; 6,432,696; 6,475,808 and 6,489,159 and PCT Publication No. WO 01/45843 A2, the disclosures of which are incorporated by reference in their entireties.

As described in detail above, the present invention provides reagents and methods for the detection of the UASMS1, UASMS2, and EXON2-FB SNPs in DNA samples obtained from individual animals. For example, using the methods of the present invention, one can determine whether a given animal has a cytosine or a thymine at the polymorphic UASMS1 locus (located at nucleotide position 207 of the ob gene promoter). Having used the methods of the invention to determine the genotype of an animal of interest at either the UASMS1, UASMS2, and/or EXON2-FB a polymorphic loci, it is a further object of the present invention to utilize this genotype information to select and/or group animals according to their genotype.

As described in the Examples, certain alleles of the UASMS1, UASMS2, and EXON2-FB SNPs are associated with the economically important trait of dairy form. For example, the present invention demonstrates that UASMS1 had the most significant association with dairy form. The C allele of the UASMS1 locus is more significantly associated with lower dairy form and the T allele is more significantly associated with higher diary form. Thus in one embodiment, where it is desirable to group animals according to dairy form, animals can be selected and grouped according to their genotype at the polymorphic UASMS1 locus. Associations between the genotypes of each of the UASMS1, UASMS2, and EXON2-FB polymorphic loci and dairy form are described in the Examples. Thus, for each of these traits, animals can be grouped according to genotype.

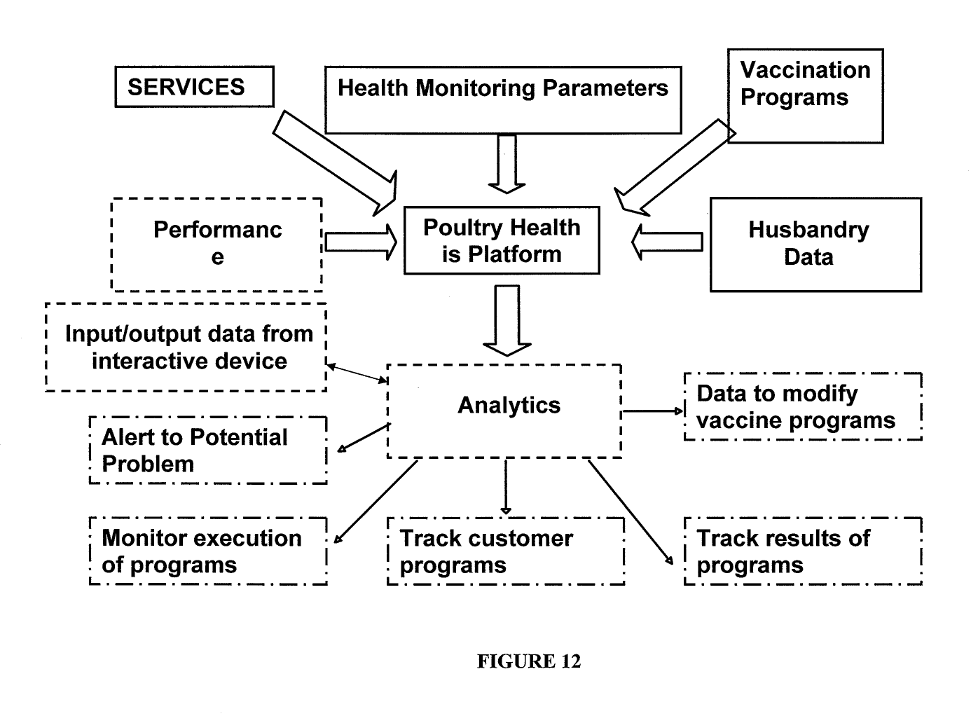

FIGS. 12, 13, 14, and 15 illustrate using flow charts how the animals may be screened for the UASMS1, UASMS2, and EXON2-FB SNPs respectively, and illustrate how the genotype information may be used to select animals to breed from and/or use for food production. The methods outlined in these flow charts are not intended to be limiting, and those skilled in the art would recognize that various aspects of these methods could be altered without affecting the overall result. FIG. 12-15 illustrate some of the phenotypic characteristics that are associated with each genotype. Other phenotypes that show some level of correlation to each genotype are shown in the Examples section.

Thus, in one embodiment, the present invention provides methods for grouping animals and methods for managing livestock production comprising grouping livestock animals, such as cattle, according to genotype of the UASMS1, UASMS2, and/or EXON2-FB polymorphic loci. The genetic selection and grouping methods of the present invention can be used in conjunction with other conventional phenotypical grouping methods such as grouping animals by visible characteristics such as weight, frame size, breed traits, and the like.

The methods of the present invention provide for selecting cattle having improved heritable traits, and can be used to optimize the performance of livestock herds in areas such as dairy form. The present invention provides methods of screening livestock to determine those more likely to develop a desired body condition by identifying the presence or absence of a polymorphism in the ob genes that is correlated with that body condition.

As described above, and in the Examples, there are various phenotypic traits with which the SNPs of the present invention are associated. Each of the phenotypic traits can be tested using the methods described in the Examples, or using any suitable methods known in the art. Using the methods of the invention, a farmer, or feed lot operator, or the like, can group cattle according to each animal's genetic propensity for a desired trait such as dairy form. as determined by SNP genotype, in addition to the present criteria he would ordinarily use for grouping. The cattle are tested to determine homozygosity or heterozygosity with respect to UASMS1, UASMS2, and EXON2-FB alleles of the ob gene so that they can be grouped such that each pen contains cattle with like genotypes.

Each pen of animals may then be fed and otherwise maintained in a manner and for a time determined by the feed lot operator to be ideal for meat production prior to slaughter, or to maximize milk production. Thus the farmer or feedlot operator is presented with opportunities for considerable efficiencies. At present, the feeder feeds all his cattle the same, incurring the same costs for each animal, and typically, with excellent management practices, perhaps 40% will grade AAA and receive the premium price for the palatability grade (depending on several other factors, such as age of animal, as we know cattle between 17-24 months of age have increased marbling compared to their younger counterparts. Approximately 55% of cattle are slaughtered at an age under 16 months, and 45% would be slaughtered at an age over 17 months). Of these, a significant number will have excess fat and will thus receive a reduced yield grade. The balance of the cattle, 60%, will grade less than AAA, and thus receive a reduced price, although the feed lot costs incurred by the operator are the same. Grouping and feeding the cattle by genotype allows the farmer to treat each group differently with a view to increasing profit.

It is contemplated that, regardless of the desirability and premium paid for any particular meat quality at any given time, providing the farmer with a more uniform group that has a predictable meat quality will provide the farmer with the opportunity to demand and receive a premium, relative to the less uniform groups of cattle presently available.

The methods of the invention are also useful in breeding programs to select for those animals having desirable phenotypes for various economically important traits, such as circulating leptin levels, feed intake, growth rate, body weight, carcass merit and composition, and milk yield. Continuous selection and breeding of animals, such as livestock, that are at least heterozygous and advantageously homozygous for a desirable polymorphism associated with, for example, improved carcass merit, would lead to a breed, line, or population having higher numbers of offspring with improved carcass merit. Thus, farmers can increase the value of their calves by using the methods of the present invention to increase the occurrence of the specific alleles in calves that are associated with economically important traits. Thus, the SNPs of the present invention can be used as selection tools in breeding programs.

For the purposes of the present invention, sequence identity or homology is determined by comparing the sequences when aligned so as to maximize overlap and identity while minimizing sequence gaps. In particular, sequence identity may be determined using any of a number of mathematical algorithms. A nonlimiting example of a mathematical algorithm used for comparison of two sequences is the algorithm of Karlin & Altschul, Proc. Natl. Acad. Sci. USA 1990; 87: 2264-2268, modified as in Karlin & Altschul, Proc. Natl. Acad. Sci. USA 1993; 90: 5873-5877.

Another example of a mathematical algorithm used for comparison of sequences is the algorithm of Myers & Miller, CABIOS 1988; 4: 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman, Proc. Natl. Acad. Sci. USA 1988; 85: 2444-2448.

Advantageous for use according to the present invention is the WU-BLAST (Washington University BLAST) version 2.0 software. WU-BLAST version 2.0 executable programs for several UNIX platforms can be downloaded from ftp://blast.wustl.edu/blast/executables. This program is based on WU-BLAST version 1.4, which in turn is based on the public domain NCBI-BLAST version 1.4 (Altschul & Gish, 1996, Local alignment statistics, Doolittle ed., Methods in Enzymology 266: 460-480; Altschul et al., Journal of Molecular Biology 1990; 215: 403-410; Gish & States, 1993; Nature Genetics 3: 266-272; Karlin & Altschul, 1993; Proc. Natl. Acad. Sci. USA 90: 5873-5877; all of which are incorporated by reference herein).

In all search programs in the suite the gapped alignment routines are integral to the database search itself. Gapping can be turned off if desired. The default penalty (Q) for a gap of length one is Q=9 for proteins and BLASTP, and Q=10 for BLASTN, but may be changed to any integer. The default per-residue penalty for extending a gap (R) is R=2 for proteins and BLASTP, and R=10 for BLASTN, but may be changed to any integer. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

Alternatively or additionally, the term “homology” or “identity”, for instance, with respect to a nucleotide or amino acid sequence, can indicate a quantitative measure of homology between two sequences. The percent sequence homology can be calculated as (N.sub.ref−N.sub.dif)*100/−N.sub.ref, wherein N.sub.dif is the total number of non-identical residues in the two sequences when aligned and wherein N.sub.ref is the number of residues in one of the sequences. Hence, the DNA sequence AGTCAGTC will have a sequence identity of 75% with the sequence AATCAATC (N_{ref}=8; N_{dif}=2). “Homology” or “identity” can refer to the number of positions with identical nucleotides or amino acids divided by the number of nucleotides or amino acids in the shorter of the two sequences wherein alignment of the two sequences can be determined in accordance with the Wilbur and Lipman algorithm (Wilbur & Lipman, Proc Natl Acad Sci USA 1983; 80:726, incorporated herein by reference), for instance, using a window size of 20 nucleotides, a word length of 4 nucleotides, and a gap penalty of 4, and computer-assisted analysis and interpretation of the sequence data including alignment can be conveniently performed using commercially available programs (e.g., Intelligenetics™. Suite, Intelligenetics Inc. CA). When RNA sequences are said to be similar, or have a degree of sequence identity or homology with DNA sequences, thymidine (T) in the DNA sequence is considered equal to uracil (U) in the RNA sequence. Thus, RNA sequences are within the scope of the invention and can be derived from DNA sequences, by thymidine (T) in the DNA sequence being considered equal to uracil (U) in RNA sequences. Without undue experimentation, the skilled artisan can consult with many other programs or references for determining percent homology.

The individual genotypic data derived from a panel or panels of SNPs of each animal or a herd or flock of animals can be recorded and associated with various other data of the animal, e.g. health information, parentage, husbandry conditions, vaccination history, herd or flock records, subsequent food safety data and the like. Such information can be forwarded to a government agency to provide traceability of an animal or meat product, or it may serve as the basis for breeding, feeding and marketing information. Once the data has or has not been associated with other data, the data is stored in an accessible database, such as, but not limited to, a computer database or a microchip implanted in the animal. The methods of the invention may provide an analysis of the input data that may be compared with parameters desired by the operator. These parameters include, but are not limited to, such as breeding goals, egg laying targets, vaccination levels of a flock or herd. If the performance or properties of the animals deviates from the desired goals, the computer-based methods may trigger an alert to allow the operator to adjust vaccination doses, medications, feed etc accordingly.

The results of the analysis provide data that is associated with the individual animal or to the herd in whole or in part from which the sample was taken. The data is then kept in an accessible database, and may or may not be associated with other data from that particular individual or from other animals.

Data obtained from individual animals may be stored in a database that can be integrated or associated with and/or cross-matched to other databases. The database along with the associated data allows information about the individual animal to be known through every stage of the animal's life, i.e., from conception to consumption of the animal product.

The accumulated data and the combination of the genetic data with other types of data of the animal provides access to information about parentage, identification of herd or flock, health information including vaccinations, exposure to diseases, feed lot location, diet and ownership changes. Information such as dates and results of diagnostic or routine tests are easily stored and attainable. Such information would be especially valuable to companies, particularly those who seek superior breeding lines.

Each animal may be provided with a unique identifier. The animal can be tagged, as in traditional tracing programs or have implant computer chips providing stored and readable data or provided with any other identification method which associates the animal with its unique identifier.

The database containing the SNP-based genotype results for each animal or the data for each animal can be associated or linked to other databases containing data, for example, which may be helpful in selecting traits for grouping or sub-grouping of an animal. For example, and not for limitation, data pertaining to animals having particular vaccination or medication protocols, can optionally be further linked with data pertaining to animals having food from certain food sources. The ability to refine a group of animals is limited only by the traits sought and the databases containing information related to those traits.

Databases that can usefully be associated with the methods of the invention include, but are not limited to, specific or general scientific data. Specific data includes, but is not limited to, breeding lines, sires, dames, and the like, other animals' genotypes, including whether or not other specific animals possess specific genes, including transgenic genetic elements, location of animals which share similar or identical genetic characteristics, and the like. General data includes, but is not limited to, scientific data such as which genes encode for specific quality characteristics, breed association data, feed data, breeding trends, and the like.

One method of the present invention includes providing the animal owner or customer with sample collection equipment, such as swabs and vials useful for collecting samples from which genetic data may be obtained. The vials are packaged in a container that is encoded with identifying indicia. Advantageously, the packaging is encoded with a bar code label. The vials are encoded with the same identifying indicia, advantageously with a matching bar code label. Optionally, the packaging contains means for sending the vials to a laboratory for analysis. The optional packaging is also encoded with identifying indicia, advantageously with a bar code label.

The method optionally includes a system wherein a database account is established upon ordering the sampling equipment. The database account identifier corresponds to the identifying indicia of the vials and the packaging. Upon shipment of the sampling equipment in fulfillment of the order, the identifying indicia are recorded in a database. Advantageously, the identifier is a bar code label which is scanned when the vials are sent. When the vials are returned to the testing facility, the identifier is again recorded and matched to the information previously recorded in the database upon shipment of the vial to the customer. Once the genotyping is completed, the information is recorded in the database and coded with the unique identifier. Test results are also provided to the customer or animal owner.

The data stored in the genotype database can be integrated with or compared to other data or databases for the purpose of identifying animals based on genetic propensities. Other data or databases include, but are not limited to, those containing information related to SNP-based DNA testing, vaccination, SUREBRED pre-conditioning program, estrus and pregnancy results, hormone levels, food safety/contamination, somatic cell counts, mastitis occurrence, diagnostic test results, milk protein levels, milk fat, vaccine status, health records, mineral levels, trace mineral levels, herd performance, and the like.

The present invention, therefore, encompasses computer-assisted methods for tracking the breeding and veterinary histories of livestock animals encompassing using a computer-based system comprising a programmed computer comprising a processor, a data storage system, an input device and an output device, and comprising the steps of generating a profile of a livestock animal by inputting into the programmed computer through the input device genotype data of the animal, wherein the genotype may be defined by a panel of at least two single nucleotide polymorphisms that predict at least one physical trait of the animal, inputting into the programmed computer through the input device welfare data of the animal, correlating the inputted welfare data with the phenotypic profile of the animal using the processor and the data storage system, and outputting a profile of the animal or group of animals to the output device.

The databases and the analysis thereof will be accessible to those to whom access has been provided. Access can be provided through rights to access or by subscription to specific portions of the data. For example, the database can be accessed by owners of the animal, the test site, the entity providing the sample to the test site, feedlot personnel, and veterinarians. The data can be provided in any form such as by accessing a website, fax, email, mailed correspondence, automated telephone, or other methods for communication. This data can also be encoded on a portable storage device, such as a microchip, that can be implanted in the animal. Advantageously, information can be read and new information added without removing the microchip from the animal.

The present invention comprises systems for performing the methods disclosed herein. Such systems comprise devices, such as computers, internet connections, servers, and storage devices for data. The present invention also provides for a method of transmitting data comprising transmission of information from such methods herein discussed or steps thereof, e.g., via telecommunication, telephone, video conference, mass communication, e.g., presentation such as a computer presentation (e.g. POWERPOINT), internet, email, documentary communication such as computer programs (e.g. WORD) and the like.

Systems of the present invention may comprise a data collection module, which includes a data collector to collect data from an animal or embryo and transmit the data to a data analysis module, a network interface for receiving data from the data analysis module, and optionally further adapted to combine multiple data from one or more individual animals, and to transmit the data via a network to other sites, or to a storage device.

More particularly, systems of the present invention comprise a data collection module, a data analysis module, a network interface for receiving data from the data analysis module, and optionally further adapted to combine multiple data from one or more individual animals, and to transmit the data via a network to other sites, and/or a storage device. For example, the data collected by the data collection module leads to a determination of the absence or presence of a SNP of a gene in the animal or embryo, and for example, such data is transmitted to a feedstock site when the feeding regimen of the animal is planned.

In one embodiment where the data is implanted on a microchip on a particular animal, the farmer can optimize the efficiency of managing the herd because the farmer is able to identify the genetic predispositions of an individual animal as well as past, present and future treatments (e.g., vaccinations and veterinarian visits). The invention, therefore also provides for accessing other databases, e.g., herd or flock data relating to genetic tests and data performed by others, by datalinks to other sites. Therefore, data from other databases can be transmitted to the central database of the present invention via a network interface for receiving data from the data analysis module of the other databases.

The invention relates to a computer system and a computer readable media for compiling data on an animal, the system containing inputted data on that animal, such as but not limited to, vaccination and medication histories, DNA testing, thyroglobulin testing, leptin, MMI (Meta Morphix Inc.), Bovine spongiform encephalopathy (BSE) diagnosis, brucellosis vaccination, FMD (foot and mouth disease) vaccination, BVD (bovine viral diarrhea) vaccination, SUREBRED pre-conditioning program, estrus and pregnancy results, tuberculosis, hormone levels, food safety/contamination, somatic cell counts, mastitis occurrence, diagnostic test results, milk protein levels, milk fat, vaccine status, health records, mineral levels, trace mineral levels, herd performance, and the like. The data of the animal can also include prior treatments as well as suggested tailored treatment depending on the genetic predisposition of that animal toward a particular disease.

The invention also provides for a computer-assisted method for improving animal production comprising using a computer system, e.g., a programmed computer comprising a processor, a data storage system, an input device and an output device, the steps of inputting into the programmed computer through the input device data comprising a breeding, veterinary, medication, diagnostic data and the like of an animal, correlating a physical characteristic predicted by the genotype using the processor and the data storage system, outputting to the output device the physical characteristic correlated to the genotype and feeding the animal a diet based upon the physical characteristic, thereby improving livestock production.

The invention further provides for a computer-assisted method for optimizing efficiency of feed lots for livestock comprising using a computer system, e.g., a programmed computer comprising a processor, a data storage system, an input device and an output device, and the steps of inputting into the programmed computer through the input device data comprising a breeding, veterinary etc history of an animal, correlating the breeding, veterinary etc histories using the processor and the data storage system, outputting to the output device the physical characteristic correlated to the genotype and feeding the animal a diet based upon the physical characteristic, thereby optimizing efficiency of feed lots for livestock.

The invention further comprehends methods of doing business by providing access to such computer readable media and/or computer systems and/or data collected from animals to users; e.g., the media and/or sequence data can be accessible to a user, for instance on a subscription basis, via the Internet or a global communication/computer network; or, the computer system can be available to a user, on a subscription basis.

In one embodiment, the invention provides for a computer system for managing livestock comprising physical characteristics and databases corresponding to one or more animals. In another embodiment, the invention provides for computer readable media for managing livestock comprising physical characteristics and veterinary histories corresponding to one or more animals. The invention further provides methods of doing business for managing livestock comprising providing to a user the computer system and media described above or physical characteristics and veterinary histories corresponding to one or more animals. The invention further encompasses methods of transmitting information obtained in any method or step thereof described herein or any information described herein, e.g., via telecommunications, telephone, mass communications, mass media, presentations, internet, email, etc.

The invention further encompasses kits useful for screening nucleic acid isolated from one or more bovine individuals for allelic variation of any one of the leptin genes, and in particular for any of the SNPs described herein, wherein the kits may comprise at least one oligonucleotide selectively hybridizing to a nucleic acid comprising any one of the one or more of which are leptin sequences described herein and instructions for using the oligonucleotide to detect variation in the nucleotide corresponding to the SNP of the isolated nucleic acid.

One embodiment of this aspect of the invention provides an oligonucleotide that specifically hybridizes to the isolated nucleic acid molecule of this aspect of the invention, and wherein the oligonucleotide hybridizes to a portion of the isolated nucleic acid molecule comprising any one of the polymorphic sites in the leptin sequences described herein.

Another embodiment of the invention is an oligonucleotide that specifically hybridizes under high stringency conditions to any one of the polymorphic sites of the leptin genes, wherein the oligonucleotide is between about 18 nucleotides and about 50 nucleotides.

In another embodiment of the invention, the oligonucleotide comprises a central nucleotide specifically hybridizing with a leptin gene polymorphic site of the portion of the nucleic acid molecule.

Another aspect of the invention is a method of identifying a leptin polymorphism in a nucleic acid sample comprising isolating a nucleic acid molecule encoding leptin or a fragment thereof and determining the nucleotide at the polymorphic site.

Another aspect of the invention is a method of screening cattle to determine those bovines more likely to exhibit a biological difference in meat quality comprising the steps of obtaining a sample of genetic material from a bovine; and assaying for the presence of a genotype in the bovine which is associated with meat quality, the genotype characterized by a polymorphism in any one of the leptin genes.

In other embodiments of this aspect of the invention, the step of assaying is selected from the group consisting of: restriction fragment length polymorphism (RFLP) analysis, minisequencing, MALD-TOF, SINE, heteroduplex analysis, single strand conformational polymorphism (SSCP), denaturing gradient gel electrophoresis (DGGE) and temperature gradient gel electrophoresis (TGGE).

In various embodiments of the invention, the method may further comprise the step of amplifying a region of the leptin gene or a portion thereof that contains the polymorphism. In other embodiments of the invention, the amplification may include the step of selecting a forward and a reverse sequence primer capable of amplifying a region of the leptin gene.

Another aspect of the invention is a computer-assisted method for predicting which livestock animals possess a biological difference in meat quality comprising: using a computer system, e.g., a programmed computer comprising a processor, a data storage system, an input device and an output device, the steps of: (a) inputting into the programmed computer through the input device data comprising a leptin genotype of an animal, (b) correlating a growth, feed intake, efficiency or carcass merit quality predicted by the leptin genotype using the processor and the data storage system and (c) outputting to the output device the meat quality correlated to the leptin genotype, thereby predicting which livestock animals possess a particular growth, feed intake, efficiency or carcass merit quality.

Yet another aspect of the invention is a method of doing business for managing livestock comprising providing to a user computer system for managing livestock comprising physical characteristics and genotypes corresponding to one or more animals or a computer readable media for managing livestock comprising physical characteristics and genotypes corresponding to one or more animals or physical characteristics and genotypes corresponding to one or more animals.

The invention will now be further described by way of the following non-limiting examples.

### EXAMPLES

### Example 1

### Leptin Marker Associations with Dairy Form

The Merial-Select Sires Dairy DNA repository was built with the following acquisitions: (1) 304 Select Sires bulls from 2004 sample (2) 278 Select Sires “large-family” or within family bulls (3) 359 Select Sires progeny test bulls—1997 to 2001; considered an unselected population (4) 28 Select Sires “random” or “across-family” bulls (5) 261 “broker” bulls for building the large/within- and across-family populations.

These 1,230 bulls were genotyped for the following markers in the leptin gene to evaluate their associations with production and type traits: (1) C207T SNP in the leptin gene promoter, denoted as UASMS1 (2) C528T SNP in the leptin gene promoter, denoted as UASMS2 (3) C1180T SNP in the leptin gene, denoted as Exon2-FB from University of Saskatchewan.

Three statistical models were used to analyze the data (as well as a goodness of fit test): (1) fixed genotype (2) allele substitution (3) haplotype.

Three different values were used to represent the phenotypes or trait measurements: (1) Standardized Transmitting Ability (STA) for dairy form (PTAs for other traits) (2) Mendelian sampling term (PTA−PA=½ Mend sampling) (3) Daughter type deviations (DTDs).

Different sample populations were utilized for the analyses based on the genotype information available for bulls and traits to be analyzed. Software was also used to check for inaccurate genotypes and to predict the most likely haplotypes.

Sample populations for the analyses involving PTAs and Mendelian sampling terms: (1) Individual marker analyses (a) 1,023—All bulls (b) 911—Young progeny test bulls born after 1995 (2) Haplotypes—844 bulls.

Sample populations for the DTD analyses included: (1) Individual marker analyses (a) 1,142—All bulls (b) 1,025—Young progeny test bulls born after 1995 (2) Haplotypes—846 bulls

All three leptin markers have significant associations with dairy form when analyzed with the “phenotypes” and populations described above.

UASMS1 had the highest statistical significance (p-values) levels, the best fit for the additive (allele substitution) model, as well as the largest effect. The C allele is associated with lower dairy form and thus, the T allele is associated with higher dairy form. Table 1 shows the results for all three “phenotypes” from UASMS1 analyses only. The bulls born after 1995 consist of four years of bulls from Select Sires that entered progeny test. This group could be considered an unselected group of bulls and if we assume they are a random sample of all bulls entering progeny test during those years for all AI companies, the results obtained from them should be relatively free of selection bias.

Genotype values presented in Table 1 would be appropriate for AI bulls, much like PTAs. These values can be doubled (see Table 2) to reflect the effect to consider in making selection decisions on individual animals (like breeding values).

TABLE 1
Association of leptin promoter marker UASMS1 with dairy form in AI bulls

| | DTD | | STA | | Mendelian sampling | |
|---|---|---|---|---|---|---|
| | All Bulls | Born > ‘95 | All Bulls | Born > ‘95 | All Bulls | Born > ‘95 |
| N | 1,142 | 1,025 | 1,023 | 1,017 | 911 | 911 |
| Allele subs. | 0.415 | 0.484 | 0.292 | 0.259 | 0.343 | 0.295 |
| Prob. value | 1.09E−09 | 5.48E−11 | 1.25E−09 | 3.78E−04 | 2.81E−11 | 9.79E−05 |
| TT* | 0.673 | 0.704 | 0.686 | −0.457 | 0.724 | −0.473 |
| CT | 0.258 | 0.219 | 0.394 | −0.716 | 0.381 | −0.768 |
| CC | −0.158 | −0.265 | 0.102 | −0.975 | 0.037 | −1.063 |
| TT** | 0.831 | 0.968 | 0.584 | 0.518 | 0.687 | 0.590 |
| CT | 0.415 | 0.484 | 0.292 | 0.259 | 0.343 | 0.295 |
| CC | 0 | 0 | 0 | 0 | 0 | 0 |

*Additive genotype estimates
**TT & CT genotypes deviated from CC

TABLE 2
Effect of UASMS1 on dairy form doubled for selection on individual animals

| | DTD | | STA | | Mendelian sampling | |
|---|---|---|---|---|---|---|
| | All Bulls | Born > ‘95 | All Bulls | Born > ‘95 | All Bulls | Born > ‘95 |
| N | 1,142 | 1,025 | 1,023 | 1,017 | 911 | 911 |
| Allele subs. | 0.831 | 0.968 | 0.584 | 0.518 | 0.687 | 0.590 |
| Prob. value | 1.09E−09 | 5.48E−11 | 1.25E−09 | 3.78E−04 | 2.81E−11 | 9.79E−05 |
| TT* | 1.346 | 1.407 | 1.371 | −0.915 | 1.449 | −0.945 |
| CT | 0.515 | 0.439 | 0.787 | −1.432 | 0.762 | −1.536 |
| CC | −0.315 | −0.530 | 0.203 | −1.950 | 0.075 | −2.126 |
| TT** | 1.661 | 1.937 | 1.168 | 1.035 | 1.374 | 1.180 |
| CT | 0.831 | 0.968 | 0.584 | 0.518 | 0.687 | 0.590 |
| CC | 0 | 0 | 0 | 0 | 0 | 0 |

*Additive genotype estimates
**TT & CT Genotypes deviated from CC

TABLE 3
Genotype and allele frequencies for UASMS1 in the
“Bulls > ‘95” sample population

| Genotype | | | Allele | |
|---|---|---|---|---|
| | N | Frequency | | Frequency |
| TT | 398 | 0.388 | T | 0.615 |
| CT | 465 | 0.454 | C | 0.385 |
| CC | 162 | 0.158 | | |
| Total | 1,025 | | | |

TABLE 4
Genotype and allele frequencies for UASMS1

| Genotype | | | Allele | |
|---|---|---|---|---|
| | N | Frequency | | Frequency |
| TT | 407 | 0.397 | T | 0.623 |
| CT | 463 | 0.452 | C | 0.377 |
| CC | 155 | 0.151 | | |
| Total | 1,025 | | | |

There is a very strong association (P<10⁻¹¹) between leptin promoter marker UASMS1 and dairy form. All analyses clearly show this strong association between UASMS1 and dairy form—recall that analyses included multiple sub-samples of the original sample population, multiple statistical models, and multiple “phenotypes.”

The difference between homozygous dairy form genotypes (CC-TT) for individual animals is as high as −1.937 STA, or 2.32 points (−1.937 STA*1.2 SD) lower for dairy form based on information in the example from Chuck Sattler. Note that most animals in the population would have a STA between ±3, and essentially all between ±4.

DuraMAX scoring system: CC=10, CT=5, and TT=1.

Dairy form/character has a high genetic correlation with BCS; however, dairy form is a better indicator trait for resistance to diseases and reproductive problems.

U.S. dairy form has a high genetic correlation with similar traits used in other countries, e.g., Canadian (0.86) and Denmark/Finland/Sweden (0.85) dairy character and UK angularity (0.88).

Genetic correlations from numerous studies indicate that cows with higher dairy form also have increased milk production, days open, and disease incidence and decreased BCS, DPR, and PL.

Most of these correlations are only slightly lower after adjusting for milk production.

Dairy form now has a negative weight in the new (2005) U.S. Holstein TPI (Type and Production Index) formula. It was not included previously (since 1976).

The preliminary study conducted in 2004 indicated that a haplotype involving UASMS1 had a significant association with PL. While we did not find that significant association in the current analyses, the trend for the effect of UASMS1 on PL and DPR are consistent with what we would expect based on the genetic correlations between dairy form and these traits.

This test should be positioned as an additional tool for producers to use in their high producing herds to assess which females are more likely to have increased durability, better reproduction, and worse disease incidence, realizing there may be some decrease in production if too much selection pressure is put just on decreasing dairy form. The U.S. Holtein Association (see below) and numerous scientists are advocating the use of dairy form in concert with production to select cows that produce at an economic level longer than average cows based on milk production, productive life, disease resistance, and reproduction. This message is supported by Dr. Tom Lawlor according to the following message (personal communication) he sent after reading about DuraMAX: “We don't want to tell people to select for lower dairy form; we do want to tell breeders to select for high producing cows that look like they do it easily, i.e., don't have extreme dairy form. It may be beneficial to give the TPI formula as an example of having positive emphasis on production along with a negative weight on dairy form.” The February 2005 TPI formula:

$$[ \frac{\text{?}}{19.4} \frac{\text{?}}{23.6} \frac{\text{?}}{.7} \frac{\text{?}}{1.8} \frac{\text{?}}{.8} \frac{\text{?}}{.85} \frac{\text{?}}{.9} \frac{\text{?}}{.13} \frac{\text{?}}{1.0} \frac{\text{?}}{1.0} ] 25 + 1548$$

$$\text{?} \text{indicates text missing or illegible when filed}$$

TABLE 5
Genotype and allele frequencies for UASMS2

| Genotype | | | Allele | |
|---|---|---|---|---|
| | N | Frequency | | Frequency |
| TT | 31 | 0.029 | T | 0.154 |
| CT | 262 | 0.249 | C | 0.846 |
| CC | 761 | 0.722 | | |
| Total | 1,054 | | | |

TABLE 6
Exon2-FB
Genotype and allele frequencies for Exon2-FB

| Genotype | | | Allele | |
|---|---|---|---|---|
| | N | Frequency | | Frequency |
| TT | 137 | 0.137 | T | 0.366 |
| CT | 458 | 0.458 | C | 0.634 |
| CC | 406 | 0.406 | | |
| Total | 1,001 | | | |

Two- and three-way combinations of markers (haplotypes) were not considered to provide a better fit of the data than single markers.

TABLE 7

Genotype and allele frequencies for
UASMS1-UASMS2-Exon2-FB

| Genotype | | Haplotype | |
|---|---|---|---|
| N | Frequency | | Frequency |

| | | | | |
|---|---|---|---|---|
| CCC/CCC | 3 | 0.004 | CCC | 0.028 |
| CCC/CCT | 7 | 0.008 | CCT | 0.343 |
| CCC/TCC | 4 | 0.005 | CTC | 0.005 |
| CCC/TCT | 25 | 0.030 | CTT | 0.003 |
| CCC/TTT | 6 | 0.007 | TCC | 0.436 |
| CCT/CCT | 120 | 0.142 | TCT | 0.028 |
| CCT/TCC | 250 | 0.296 | TTC | 0.152 |
| CCT/TTC | 84 | 0.099 | TTT | 0.004 |
| CTC/TCT | 9 | 0.011 | | |
| CTT/TCC | 5 | 0.006 | | |
| TCC/TCC | 178 | 0.210 | | |
| TCC/TCT | 6 | 0.007 | | |
| TCC/TTC | 116 | 0.137 | | |
| TCT/TCT | 1 | 0.001 | | |
| TCT/TTC | 6 | 0.007 | | |
| TTC/TTC | 26 | 0.031 | | |
| Total | 846 | | | |

The markers UASMS1, UASMS2, and Exon2-FB are all associated with the phenotypic trait of dairy form individually and in combination. Results indicated that they are all essentially equivalent in effect. Any one of the markers were put either individually or in combination to analyze dairy form. Table 8 indicates that all essentially give the same result.

TABLE 8
Estimate of Genotype Effect.

| | | Dairy form STA | | |
|---|---|---|---|---|
| Marker | | LSM | se | Prob. |
| UASMS1 | CC | 0.5 | 0.30 | 0.01 |
| | CT | 0.7 | 0.26 | 0.02 |
| | TT | 1.1 | 0.26 | |
| UASMS2 | CC | 0.8 | 0.25 | 0.91 |
| | CT | 1.0 | 0.27 | 0.58 |
| | TT | 0.7 | 0.56 | |
| Exon2fb | CC | 1.1 | 0.26 | 0.01 |
| | CT | 0.7 | 0.26 | 0.31 |
| | TT | 0.5 | 0.28 | |

TABLE 9
Dairy Form Statistical Analysis

| All Bulls | | Young Bulls-Born after 1995 | |
|---|---|---|---|
| | Dairy Form | | Dairy Form |
| UASMS1 (PTA) | | UASMS1 (PTA) | |
| N | 1023 | N | 911 |
| a (T-C) | 0.584224344 | a (T-C) | 0.686778002 |
| SE(a) | 0.095300372 | SE(a) | 0.101896708 |
| ESS[A] | 948.562849 | ESS[A] | 847.8451791 |
| VA | 0.929052741 | VA | 0.932722969 |
| ESS[R] | 983.4777229 | ESS[R] | 890.2158279 |
| F1, N-2 | 37.58115367 | F1, N-2 | 45.42683108 |
| P(1, N-2) | 1.25113E−09 | P(1, N-2) | 2.81153E−11 |
| GOF-F1, N-3 | 0.097919354 | GOF-F1, N-3 | 0.051338072 |
| P(1, N-3) | 0.754404891 | P(1, N-3) | 0.820802071 |
| ESS[S] | 948.4717963 | ESS[S] | 847.7972449 |
| F2, N-2 | 18.82293454 | F2, N-2 | 22.71538012 |
| P(2, N-2) | 9.38437E−09 | P(2, N-2) | 2.36288E−10 |
| TT-S (n = 407) | 0.691919935 | TT-S (n = 368) | 0.719566402 |
| CT-S (n = 463) | 0.382832396 | CT-S (n = 406) | 0.389358943 |
| CC-S (n = 155) | 0.117864893 | CC-S (n = 138) | 0.024780551 |
| TT-A (n = 407) | 0.685745297 | TT-A (n = 368) | 0.724257654 |
| CT-A (n = 463) | 0.393633125 | CT-A (n = 406) | 0.380868652 |
| CC-A (n = 155) | 0.101520953 | CC-A (n = 138) | 0.037479651 |
| UASMS1 (M) | | UASMS1 (M) | |
| N | 1017 | N | 911 |
| a (T-C) | 0.517573536 | a (T-C) | 0.590198334 |
| SE(a) | 0.145105873 | SE(a) | 0.150822516 |
| ESS[A] | 546.546613 | ESS[A] | 464.3747724 |
| VA | 0.538469569 | VA | 0.510863336 |
| ESS[R] | 553.3973187 | ESS[R] | 472.1976823 |
| F1, N-2 | 12.72254941 | F1, N-2 | 15.31311674 |
| P(1, N-2) | 0.000378135 | P(1, N-2) | 9.78768E−05 |
| GOF-F1, N-3 | 0.494969871 | GOF-F1, N-3 | 0.800098713 |
| P(1, N-3) | 0.481880206 | P(1, N-3) | 0.371300893 |
| ESS[S] | 546.2799541 | ESS[S] | 463.9659414 |
| F2, N-2 | 6.605594481 | F2, N-2 | 8.054923946 |
| P(2, N-2) | 0.00141168 | P(2, N-2) | 0.000340744 |
| TT-S (n = 407) | −0.436255876 | TT-S (n = 368) | −0.44526339 |
| CT-S (n = 463) | −0.753143269 | CT-S (n = 406) | −0.8173545 |
| CC-S (n = 155) | −0.91902352 | CC-S (n = 138) | −0.98868876 |
| TT-A (n = 407) | −0.457389459 | TT-A (n = 368) | −0.47266448 |
| CT-A (n = 463) | −0.716176227 | CT-A (n = 406) | −0.76776365 |
| CC-A (n = 155) | −0.974962995 | CC-A (n = 138) | −1.06286281 |
| UASMS2 (PTA) | | UASMS2 (PTA) | |
| N | 1052 | N | 940 |
| a (C-T) | −0.400541581 | a (C-T) | −0.466249 |
| SE(a) | 0.129502078 | SE(a) | 0.139848089 |
| ESS[A] | 1036.709085 | ESS[A] | 943.2855322 |
| VA | 0.987341985 | VA | 1.005634896 |
| ESS[R] | 1046.154245 | ESS[R] | 954.4635062 |
| F1, N-2 | 9.566250326 | F1, N-2 | 11.11534019 |
| P(1, N-2) | 0.002034213 | P(1, N-2) | 0.000889692 |
| GOF-F1, N-3 | 1.750232458 | GOF-F1, N-3 | 0.961262196 |
| P(1, N-3) | 0.186135819 | P(1, N-3) | 0.327122147 |
| ESS[S] | 1034.98224 | ESS[S] | 942.3188135 |
| F2, N-2 | 5.661658969 | F2, N-2 | 6.038071669 |
| P(2, N-2) | 0.003583739 | P(2, N-2) | 0.002479908 |
| CC-S (n = 761) | 0.376885306 | CC-S (n = 683) | 0.370197776 |
| CT-S (n = 262) | 0.635747846 | CT-S (n = 230) | 0.650805099 |
| TT-S (n = 31) | 0.574304798 | TT-S (n = 28) | 0.675448895 |
| CC-A (n = 761) | 0.385516537 | CC-A (n = 683) | 0.377056923 |
| CT-A (n = 262) | 0.585787328 | CT-A (n = 230) | 0.610181423 |
| TT-A (n = 31) | 0.786058118 | TT-A (n = 28) | 0.843305924 |
| UASMS2 (M) | | UASMS2 (M) | |
| N | 1046 | N | 940 |
| a (C-T) | −0.290667258 | a (C-T) | −0.4370108 |
| SE(a) | 0.195151443 | SE(a) | 0.204683043 |
| ESS[A] | 585.1917199 | ESS[A] | 505.1656857 |
| VA | 0.560528467 | VA | 0.538556168 |
| ESS[R] | 586.4352216 | ESS[R] | 507.6206864 |
| F1, N-2 | 2.218445134 | F1, N-2 | 4.558485871 |
| P(1, N-2) | 0.136672359 | P(1, N-2) | 0.033014581 |
| GOF-F1, N-3 | 6.462952515 | GOF-F1, N-3 | 4.354734556 |
| P(1, N-3) | 0.011158338 | P(1, N-3) | 0.037176021 |
| ESS[S] | 581.5879088 | ESS[S] | 502.8287745 |
| F2, N-2 | 4.346503068 | F2, N-2 | 4.464761871 |
| P(2, N-2) | 0.013187221 | P(2, N-2) | 0.011753031 |
| CC-S (n = 761) | −0.72866415 | CC-S (n = 683) | −0.79510915 |
| CT-S (n = 262) | −0.414044576 | CT-S (n = 230) | −0.42895236 |
| TT-S (n = 31) | −1.024866462 | TT-S (n = 28) | −0.85873334 |
| CC-A (n = 761) | −0.703726405 | CC-A (n = 683) | −0.77378012 |
| CT-A (n = 262) | −0.558392776 | CT-A (n = 230) | −0.55527472 |
| TT-A (n = 31) | −0.413059147 | TT-A (n = 28) | −0.33676932 |
| EXON_2_FB_(PTA) | | EXON_2_FB_(PTA) | |
| N | 999 | N | 889 |
| a (C-T) | 0.526557316 | a (C-T) | 0.621514008 |
| SE(a) | 0.099881516 | SE(a) | 0.1079335 |
| ESS[A] | 960.9574334 | ESS[A] | 870.1299739 |
| VA | 0.96384898 | VA | 0.980980805 |
| ESS[R] | 987.7448012 | ESS[R] | 902.6574096 |
| F1, N-2 | 27.79207985 | F1, N-2 | 33.15807563 |
| P(1, N-2) | 1.65598E−07 | P(1, N-2) | 1.17062E−08 |
| GOF-F1, N-3 | 0.212182437 | GOF-F1, N-3 | 0.008548359 |
| P(1, N-3) | 0.645162739 | P(1, N-3) | 0.926355588 |
| ESS[S] | 960.7527598 | ESS[S] | 870.1215787 |
| F2, N-2 | 13.99115066 | F2, N-2 | 16.56478064 |
| P(2, N-2) | 1.01725E−06 | P(2, N-2) | 8.65169E−08 |
| CC-S (n = 406) | 0.652672359 | CC-S (n = 368) | 0.667254744 |
| CT-S (n = 458) | 0.364390265 | CT-S (n = 402) | 0.36188902 |
| TT-S (n = 137) | 0.143951893 | TT-S (n = 120) | 0.041724428 |
| CC-A (n = 406) | 0.643633143 | CC-A (n = 368) | 0.669159979 |
| CT-A (n = 458) | 0.380354485 | CT-A (n = 402) | 0.358402976 |
| TT-A (n = 137) | 0.117075827 | TT-A (n = 120) | 0.047645972 |
| EXON_2_FB_(M) | | EXON_2_FB_(M) | |
| N | 993 | N | 889 |
| a (C-T) | 0.467033647 | a (C-T) | 0.495754847 |
| SE(a) | 0.153222858 | SE(a) | 0.16014668 |
| ESS[A] | 561.9523889 | ESS[A] | 478.9029287 |
| VA | 0.567055892 | VA | 0.53991311 |
| ESS[R] | 567.2207446 | ESS[R] | 484.0768752 |
| F1, N-2 | 9.29071676 | F1, N-2 | 9.582924332 |
| P(1, N-2) | 0.002364263 | P(1, N-2) | 0.002025633 |
| GOF-F1, N-3 | 0.827697877 | GOF-F1, N-3 | 0.709283732 |
| P(1, N-3) | 0.363160495 | P(1, N-3) | 0.399908969 |
| ESS[S] | 561.4829563 | ESS[S] | 478.5198516 |
| F2, N-2 | 5.058399645 | F2, N-2 | 5.144533619 |
| P(2, N-2) | 0.006520826 | P(2, N-2) | 0.006006432 |
| CC-S (n = 406) | −0.475706072 | CC-S (n = 368) | −0.52187541 |
| CT-S (n = 458) | −0.784955945 | CT-S (n = 402) | −0.84258957 |
| TT-S (n = 137) | −0.888713584 | TT-S (n = 120) | −0.96336952 |
| CC-A (n = 406) | −0.503085006 | CC-A (n = 368) | −0.54761533 |
| CT-A (n = 458) | −0.736601829 | CT-A (n = 402) | −0.79549276 |
| TT-A (n = 137) | −0.970118653 | TT-A (n = 120) | −1.04337018 |
| Haplotypes_(PTA) | | Haplotypes_(PTA) | |
| N | 844 | N | 740 |
| ESS[F] | 772.3768153 | ESS[F] | 686.8756033 |
| ESS[A] | 780.20186 | ESS[A] | 693.0364487 |
| ESS[M] | 810.8609167 | ESS[M] | 731.3285147 |
| F7, N-8 | 4.693102551 | F7, N-8 | 5.777843366 |
| P(7, N-8) | 3.54471E−05 | P(7, N-8) | 1 .56826E−06 |
| GOF-F8, N-16 | 5.156944657 | GOF-F8, N-16 | 5.856938959 |
| P(8, N-16) | 2.69155E−06 | P(8, N-16) | 2.84563E−07 |
| F15, N-16 | 2.750370484 | F15, N-16 | 3.123700778 |
| P(15, N-16) | 0.00037114 | P(15, N-16) | 5.70724E−05 |
| EHap12 | 777.592657 | EHap12 | 691.5918598 |
| EHap13 | 779.4537178 | EHap13 | 690.465808 |
| EHap23 | 780.7991796 | EHap23 | 695.1446425 |
| F(7or9, N-16) | 0.621273738 | F(7or9, N-16) | 0.540606965 |
| P(7or9, 16) | 0.779553892 | P(7or9, 16) | 0.803912514 |
| E1 | 781.6714542 | E1 | 756.7636237 |
| E2 | 801.1041591 | E2 | 720.7852407 |
| E3 | 784.7658833 | E3 | 698.1937514 |
| F(4or6, N-7or9) | 1.097603354 | F(4or6, N-7or9) | 1.363602801 |
| P(4or6, N-7or9) | 0.356504294 | P(4or6, N-7or9) | 0.226727501 |
| F(13, N-16) | 0.766461374 | F(13, N-16) | 0.917682593 |
| P(13, N-16) | 0.696373572 | P(13, N-16) | 0.534028309 |
| Haplotypes_(M) | | Haplotypes_(M) | |
| N | 838 | N | 740 |
| ESS[F] | 444.6586479 | ESS[F] | 372.7199799 |
| ESS[A] | 453.3948635 | ESS[A] | 378.8993081 |
| ESS[M] | 461.2827549 | ESS[M] | 387.3084047 |
| F7, N-8 | 2.062834455 | F7, N-8 | 2.320804566 |
| P(7, N-8) | 0.045157313 | P(7, N-8) | 0.024018672 |
| GOF-F8, N-16 | 3.84143432 | GOF-F8, N-16 | 3.542210015 |
| P(8, N-16) | 0.000188543 | P(8, N-16) | 0.000491304 |
| F15, N-16 | 2.048764971 | F15, N-16 | 1.889178674 |
| P(15, N-16) | 0.010531203 | P(15, N-16) | 0.021286309 |
| EHap12 | 449.9079443 | EHap12 | 376.6952424 |
| EHap13 | 453.8840894 | EHap13 | 377.9634798 |
| EHap23 | 451.0299943 | EHap23 | 378.2698085 |
| F(7or9, N-16) | 1.078210754 | F(7or9, N-16) | 0.857984014 |
| P(7or9, 16) | 0.376324007 | P(7or9, 16) | 0.562793749 |
| E1 | 455.2099541 | E1 | 388.6576402 |
| E2 | 455.8523617 | E2 | 382.2546852 |
| E3 | 456.1216783 | E3 | 382.183276 |
| F(4or6, N-7or9) | 2.448262039 | F(4or6, N-7or9) | 2.669750127 |
| P(4or6, N-7or9) | 0.044902896 | P(4or6, N-7or9) | 0.031230909 |
| F(13, N-16) | 1.500403086 | F(13, N-16) | 1.414018114 |
| P(13, N-16) | 0.111044726 | P(13, N-16) | 0.146810959 |

### Example 2

### Data Treatment

FIG. 12 shows a flowchart of the input of data and the output of results from the analysis and correlation of the data pertaining to the breeding, veterinarian histories and performance requirements of a group of animals such as from bovines. The flowchart illustrated in FIG. 12 further indicate the interactive flow of data from the computer-assisted device to a body of students learning the use of the method of the invention and the correlation of such interactive data to present an output as a pie-chart indicating the progress of the class. The flowchart further indicates modifications of the method of the invention in accordance with the information received from the students to advance the teaching process or optimize the method to satisfy the needs of the students.

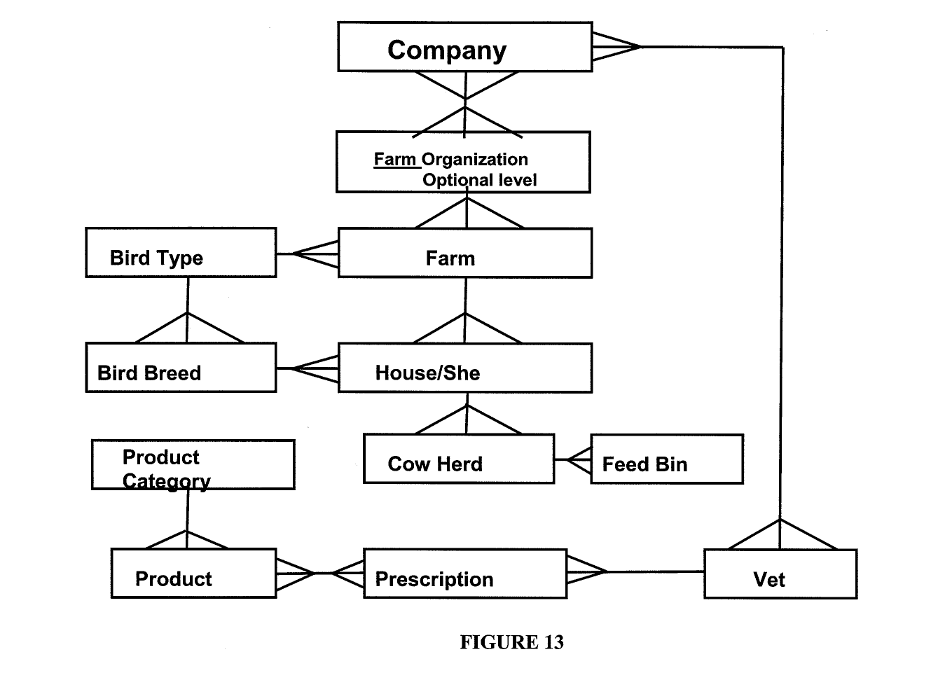

FIG. 13 illustrates potential relationships between the data elements to be entered into the system. Unidirectional arrows indicate, for example, that a house or shed is typically owned by only one farm, whereas a farm may own several houses or sheds. Similarly, a prescription may include have several veterinarian products.

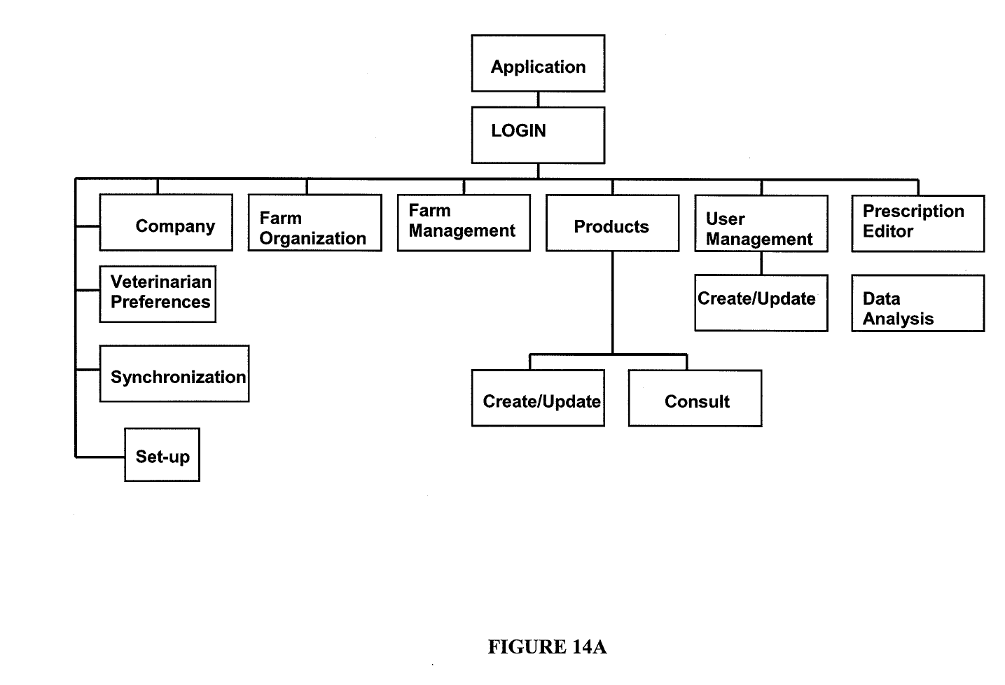

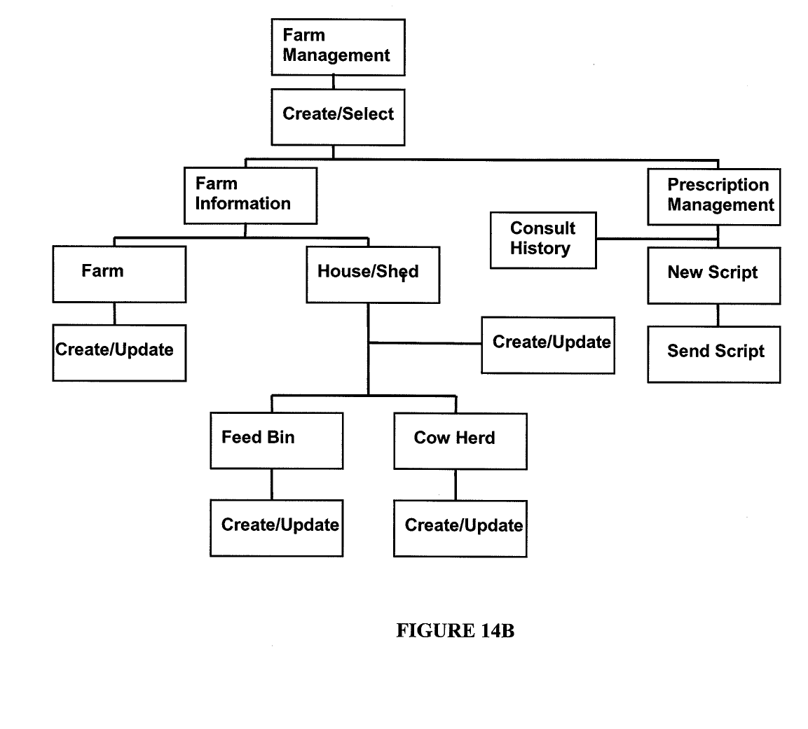

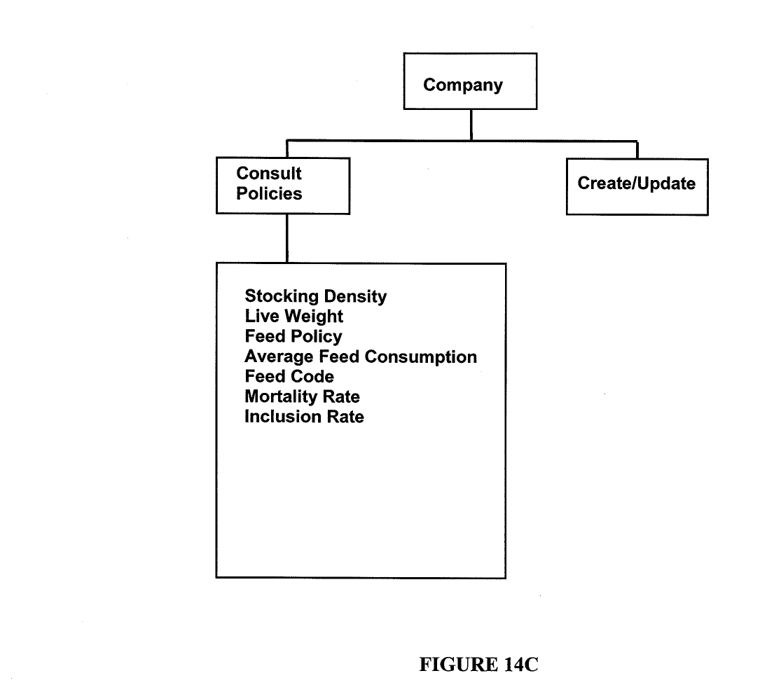

FIG. 14A illustrates the flow of events in the use of the portable computer-based system for data entry on the breeding and rearing of a herd of cows. FIG. 14B illustrates the flow of events through the sub-routines related to data entry concerning farm management. FIG. 14C illustrates the flow of events through the sub-routines related to data entry concerning data specific to a company.

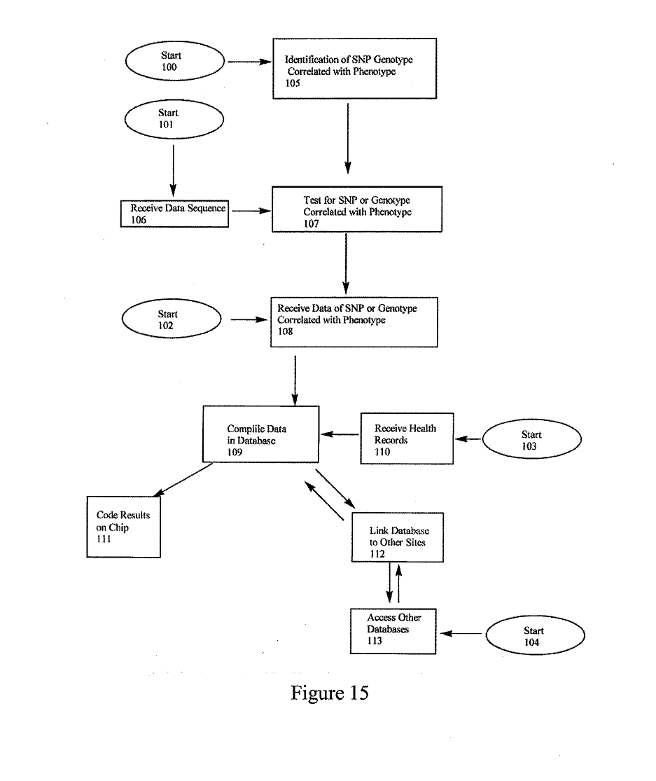

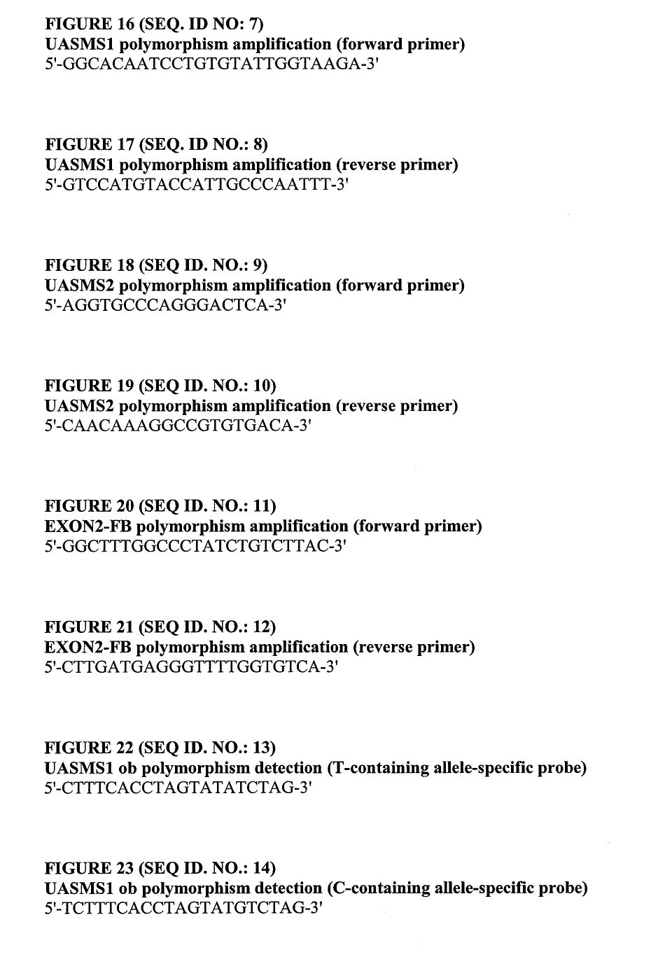

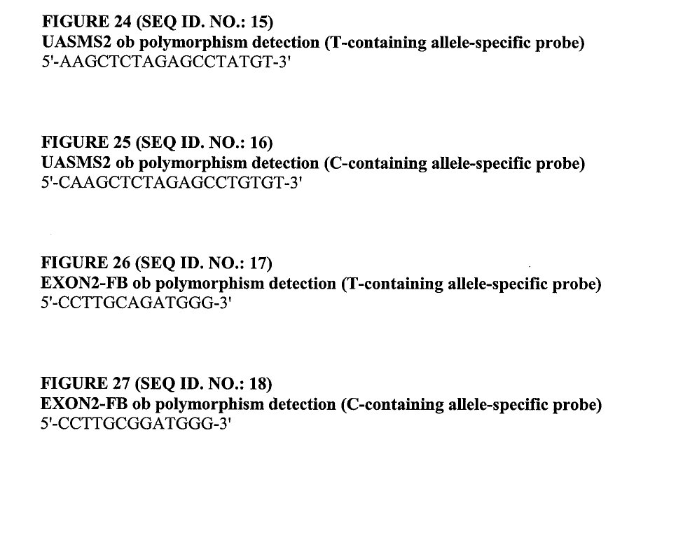

FIG. 15 illustrates a flow chart of the input of data and the output of results from the analysis and the correlation of the data pertaining to the breeding, veterinarian histories, and performance requirements of a group of animals.

### Example 3

### Productive Life

Productive life is time in the milking herd before removal by voluntary culling, involuntary culling, or death. Credits for each month in milk are obtained from standard lactation curves and are then summed across all lactations. Diminishing credits within lactation give cows more credit for beginning a new lactation than for continuing to milk in the previous lactation. Cows get 8 months credit for 305-d first lactation records, 10 months credit for second lactations, 10.2 months credit for third and later lactations, partial credits for shorter records, and extra credits for longer records. See, e.g., VanRaden, 2001. J. Dairy Sci. et al. 1993. J. Dairy Sci. 76:2758 and VanRaden et al. 2007. J. Dairy Sci. 90:2434.

The rationale for marker data analyses is if a genetic marker is a causal mutation or very closely linked to a causal mutation that affects a particular trait, then a significant difference is expected in the mean value for the trait between individuals that inherited alternative forms of the mutation from their parents.

Statistical analysis or productive life involves (a) analyses of individual gene markers—one by one—to associate genotypes to predictive transmitting activity (PTA) or daughter yield derivation (DYD), (b) multiple-marker, additive effect models to build a panel of synergistic effects with markers that showed individual association with the trait, (c) testing the significance of the panel and derive an “estimate” of the effect by looking at the additive effect of the markers and (d) comparing the “estimate” with the actual results for bulls in the population with those genotype combination scores.

An example of deriving the estimate of the effect on PTA is as follows. Suppose for Gene #1, all bulls with AA genotype: PTA for milk averages 500 lbs, all bulls with AT genotype: PTA for milk averages 1000 lbs and all bulls with TT genotype: PTA for milk averages 1500 lbs. Therefore for Gene #1, adding a “T” adds 500 lbs of milk. For Gene # 2, all bulls with CC genotype: PTA for milk averages 500 lbs, all bulls with CG genotype: PTA for milk averages 1000 lbs and all bulls with GG genotype: PTA for milk averages 1500 lbs. Therefore for Gene #2, adding a “G” adds 500 lbs. When the effects of Genes #1 and #2 are combined, the scores and estimates are derived. For example, for a genotype AA CC, of the score is 1 and the estimate is 0. For a genotype of AT CC/AA CG, the score is 2 and the estimate is 500. For a genotype of TT CC/AT CG/AA GG, the score is 3 and the estimate is 1000. For a genotype of TT CG/AT GG, the score is 4 and the estimate is 1500. For a genotype of TT GG, the score is 5 and the estimate is 2000. The estimate is the difference from score 1.

Variance measures the proportion of the variance in the dependent variable (trait) explained by the effect of the markers.

TABLE 10
Variance explained
$\% Variance = \frac{\begin{matrix}Residual Variance \\ ( no markers )\end{matrix} - \begin{matrix}Residual Variance \\ ( with markers )\end{matrix}}{Variance in the trait}$

| | # | Ani- | | % | |
|---|---|---|---|---|---|
| Trait | Markers | mals | Estimate | Variance | P-value |
| Milk Yield | 5 | 1742 | 1873 | 12.3 | 3.3E−10 |
| Fat Yield | 7 | 1742 | 70 | 17.8 | 2.9E−12 |
| Fat Percent | 5 | 1742 | 0.44 | 32.7 | 6.5E−35 |
| Protein Yield | 5 | 1742 | 34 | 10.0 | 7.5E−07 |
| Protein Percent | 4 | 1742 | 0.21 | 22.2 | 3.8E−37 |
| Productive Life | 4 | 1741 | 2.95 | 6.2 | 4.5E−07 |
| Daughter Preg. Rate | 4 | 1742 | 2.46 | 8.0 | 1.0E−05 |
| Fluid Merit Dollars | 4 | 1783 | 194 | 5.0 | 2.8E−05 |
| Net Merit Dollars | 4 | 1783 | 183 | 6.5 | 4.7E−08 |
| Cheese Merit | 3 | 1783 | 155 | 5.6 | 2.1E−07 |
| Dollars | | | | | |

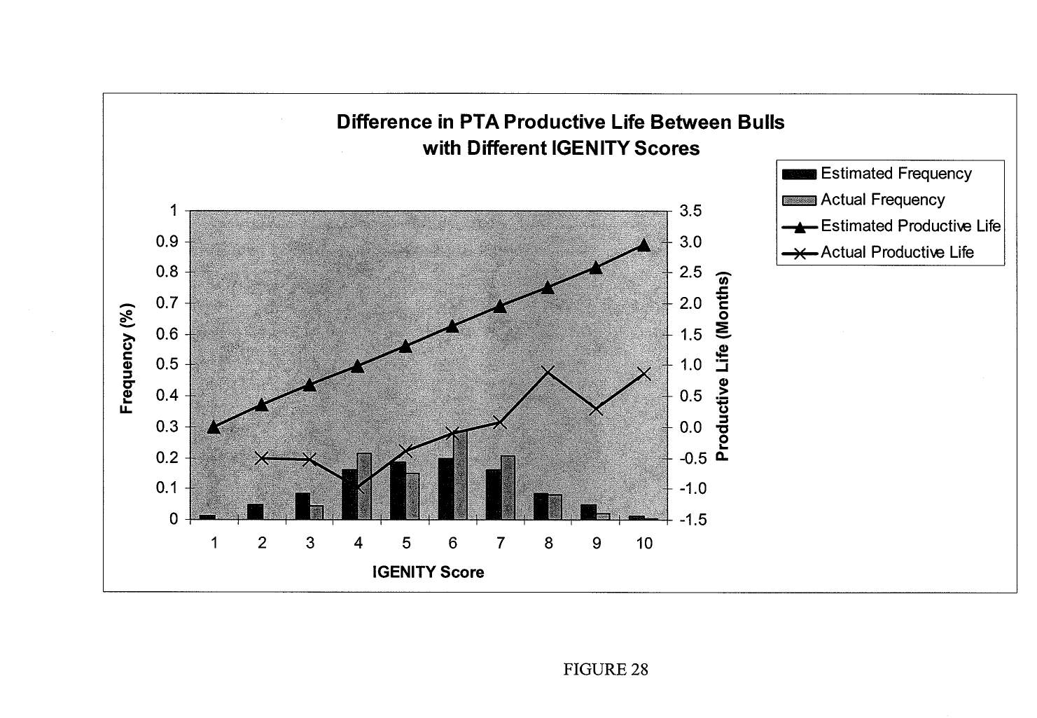

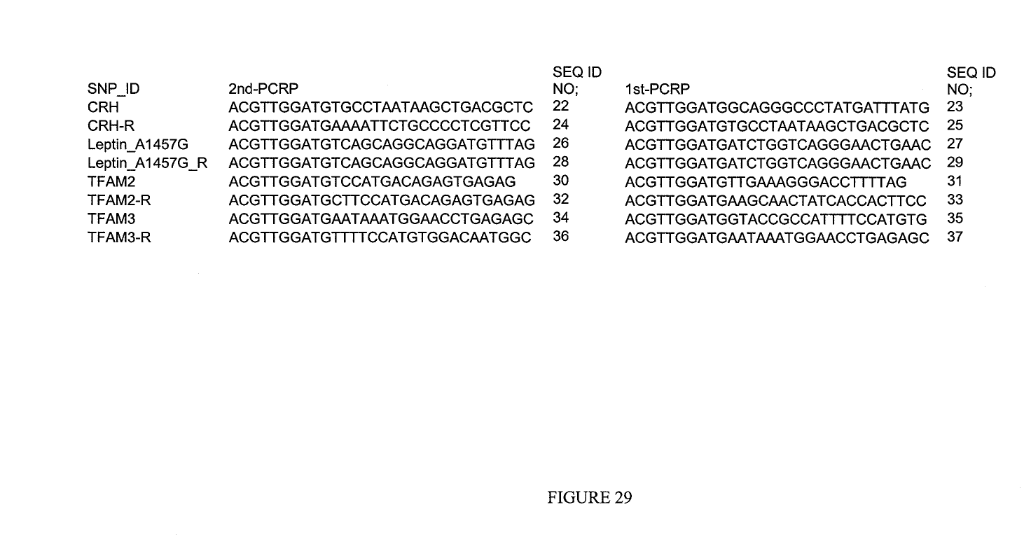

FIG. 28 depicts the difference in PTA productive life between bulls with different scores.

To summarize, analysis of productive life is a powerful tool that is predictive of future performance and can be used at birth. The scores derived from this population accurately reflect the means and matings can be targeted to increase frequency of rare alleles. Marker panels are very instructive as good phenotypes equal good panels. Excellent progress is made on elusive traits as more markers are added. With this amount of variance explained, this should be used in SMS, and female matings (eg. heifers with sexed semen) performed with this information added. Such a tool can be used much more to differentiate select sires and its evaluators from everyone else.

In an advantageous embodiment, a panel of the leptin A1457G, CRH and TFAM SNPs disclosed herein is predictive of productive life.

TABLE 11
Predicted transmitting abilities (PTA's) for productive life (PL)

| Label | Allele | Genotype | Estimate | StdErr | DF | tValue | Probt |
|---|---|---|---|---|---|---|---|
| A1457G | G | 0.7546955 | 0.377348 | 0.113096 | 677 | 3.336524 | 0.000895 |
| CRH | T | 0.755663 | 0.377831 | 0.113748 | 677 | 3.321658 | 0.000943 |
| TFAM2 | A | 0.9388761 | 0.469438 | 0.134392 | 677 | 3.493043 | 0.000509 |
| TFAM3 | G | 0.5361206 | 0.26806 | 0.14484 | 677 | 1.850734 | 0.064643 |
| PTA_PL | | 2.9853552 | 1.492678 | 0.294221 | 677 | 5.073319 | 5.05E−07 |

As shown in the genotype column of Table 11, a panel of leptin A1457G, CRH and TFAM SNPs accounts for about three (3) months of predictive life in accordance with a PTA analysis. Table 12 (below) presents a more detailed statistical analysis of the panel.

| A1457G | A1457G | CRH | CRH | TFAM2 | TFAM2 | TFAM3 | TFAM3 | PTA_PL |
|---|---|---|---|---|---|---|---|---|
| GG | 0.754696 | TT | 0.755663 | AA | 0.938876 | GG | 0.536121 | 2.985355 |
| GG | 0.754696 | TT | 0.755663 | AA | 0.938876 | AG | 0.26806 | 2.717295 |
| AG | 0.377348 | TT | 0.755663 | AA | 0.938876 | GG | 0.536121 | 2.608007 |
| GG | 0.754696 | CT | 0.377831 | AA | 0.938876 | GG | 0.536121 | 2.607524 |
| GG | 0.754696 | TT | 0.755663 | AC | 0.469438 | GG | 0.536121 | 2.515917 |
| GG | 0.754696 | TT | 0.755663 | AA | 0.938876 | AA | 0 | 2.449235 |
| AG | 0.377348 | TT | 0.755663 | AA | 0.938876 | AG | 0.26806 | 2.339947 |
| GG | 0.754696 | CT | 0.377831 | AA | 0.938876 | AG | 0.26806 | 2.339463 |
| GG | 0.754696 | TT | 0.755663 | AC | 0.469438 | AG | 0.26806 | 2.247857 |
| AA | 0 | TT | 0.755663 | AA | 0.938876 | GG | 0.536121 | 2.23066 |
| AG | 0.377348 | CT | 0.377831 | AA | 0.938876 | GG | 0.536121 | 2.230176 |
| GG | 0.754696 | CC | 0 | AA | 0.938876 | GG | 0.536121 | 2.229692 |
| AG | 0.377348 | TT | 0.755663 | AC | 0.469438 | GG | 0.536121 | 2.138569 |
| GG | 0.754696 | CT | 0.377831 | AC | 0.469438 | GG | 0.536121 | 2.138086 |
| AG | 0.377348 | TT | 0.755663 | AA | 0.938876 | AA | 0 | 2.071887 |
| GG | 0.754696 | CT | 0.377831 | AA | 0.938876 | AA | 0 | 2.071403 |
| GG | 0.754696 | TT | 0.755663 | CC | 0 | GG | 0.536121 | 2.046479 |
| GG | 0.754696 | TT | 0.755663 | AC | 0.469438 | AA | 0 | 1.979797 |
| AA | 0 | TT | 0.755663 | AA | 0.938876 | AG | 0.26806 | 1.962599 |
| AG | 0.377348 | CT | 0.377831 | AA | 0.938876 | AG | 0.26806 | 1.962116 |
| GG | 0.754696 | CC | 0 | AA | 0.938876 | AG | 0.26806 | 1.961632 |
| AG | 0.377348 | TT | 0.755663 | AC | 0.469438 | AG | 0.26806 | 1.870509 |
| GG | 0.754696 | CT | 0.377831 | AC | 0.469438 | AG | 0.26806 | 1.870025 |
| AA | 0 | CT | 0.377831 | AA | 0.938876 | GG | 0.536121 | 1.852828 |
| AG | 0.377348 | CC | 0 | AA | 0.938876 | GG | 0.536121 | 1.852344 |
| GG | 0.754696 | TT | 0.755663 | CC | 0 | AG | 0.26806 | 1.778419 |
| AA | 0 | TT | 0.755663 | AC | 0.469438 | GG | 0.536121 | 1.761222 |
| AG | 0.377348 | CT | 0.377831 | AC | 0.469438 | GG | 0.536121 | 1.760738 |
| GG | 0.754696 | CC | 0 | AC | 0.469438 | GG | 0.536121 | 1.760254 |
| AA | 0 | TT | 0.755663 | AA | 0.938876 | AA | 0 | 1.694539 |
| AG | 0.377348 | CT | 0.377831 | AA | 0.938876 | AA | 0 | 1.694055 |
| GG | 0.754696 | CC | 0 | AA | 0.938876 | AA | 0 | 1.693572 |
| AG | 0.377348 | TT | 0.755663 | CC | 0 | GG | 0.536121 | 1.669131 |
| GG | 0.754696 | CT | 0.377831 | CC | 0 | GG | 0.536121 | 1.668648 |
| AG | 0.377348 | TT | 0.755663 | AC | 0.469438 | AA | 0 | 1.602449 |
| GG | 0.754696 | CT | 0.377831 | AC | 0.469438 | AA | 0 | 1.601965 |
| AA | 0 | CT | 0.377831 | AA | 0.938876 | AG | 0.26806 | 1.584768 |
| AG | 0.377348 | CC | 0 | AA | 0.938876 | AG | 0.26806 | 1.584284 |
| GG | 0.754696 | TT | 0.755663 | CC | 0 | AA | 0 | 1.510359 |
| AA | 0 | TT | 0.755663 | AC | 0.469438 | AG | 0.26806 | 1.493161 |
| AG | 0.377348 | CT | 0.377831 | AC | 0.469438 | AG | 0.26806 | 1.492678 |
| GG | 0.754696 | CC | 0 | AC | 0.469438 | AG | 0.26806 | 1.492194 |
| AA | 0 | CC | 0 | AA | 0.938876 | GG | 0.536121 | 1.474997 |
| AG | 0.377348 | TT | 0.755663 | CC | 0 | AG | 0.26806 | 1.401071 |
| GG | 0.754696 | CT | 0.377831 | CC | 0 | AG | 0.26806 | 1.400587 |
| AA | 0 | CT | 0.377831 | AC | 0.469438 | GG | 0.536121 | 1.38339 |
| AG | 0.377348 | CC | 0 | AC | 0.469438 | GG | 0.536121 | 1.382906 |
| AA | 0 | CT | 0.377831 | AA | 0.938876 | AA | 0 | 1.316708 |
| AG | 0.377348 | CC | 0 | AA | 0.938876 | AA | 0 | 1.316224 |
| AA | 0 | TT | 0.755663 | CC | 0 | GG | 0.536121 | 1.291784 |
| AG | 0.377348 | CT | 0.377831 | CC | 0 | GG | 0.536121 | 1.2913 |
| GG | 0.754696 | CC | 0 | CC | 0 | GG | 0.536121 | 1.290816 |
| AA | 0 | TT | 0.755663 | AC | 0.469438 | AA | 0 | 1.225101 |
| AG | 0.377348 | CT | 0.377831 | AC | 0.469438 | AA | 0 | 1.224617 |
| GG | 0.754696 | CC | 0 | AC | 0.469438 | AA | 0 | 1.224134 |
| AA | 0 | CC | 0 | AA | 0.938876 | AG | 0.26806 | 1.206936 |
| AG | 0.377348 | TT | 0.755663 | CC | 0 | AA | 0 | 1.133011 |
| GG | 0.754696 | CT | 0.377831 | CC | 0 | AA | 0 | 1.132527 |
| AA | 0 | CT | 0.377831 | AC | 0.469438 | AG | 0.26806 | 1.11533 |
| AG | 0.377348 | CC | 0 | AC | 0.469438 | AG | 0.26806 | 1.114846 |
| AA | 0 | TT | 0.755663 | CC | 0 | AG | 0.26806 | 1.023723 |
| AG | 0.377348 | CT | 0.377831 | CC | 0 | AG | 0.26806 | 1.02324 |
| GG | 0.754696 | CC | 0 | CC | 0 | AG | 0.26806 | 1.022756 |
| AA | 0 | CC | 0 | AC | 0.469438 | GG | 0.536121 | 1.005559 |
| AA | 0 | CC | 0 | AA | 0.938876 | AA | 0 | 0.938876 |
| AA | 0 | CT | 0.377831 | CC | 0 | GG | 0.536121 | 0.913952 |
| AG | 0.377348 | CC | 0 | CC | 0 | GG | 0.536121 | 0.913468 |
| AA | 0 | CT | 0.377831 | AC | 0.469438 | AA | 0 | 0.84727 |
| AG | 0.377348 | CC | 0 | AC | 0.469438 | AA | 0 | 0.846786 |
| AA | 0 | TT | 0.755663 | CC | 0 | AA | 0 | 0.755663 |
| AG | 0.377348 | CT | 0.377831 | CC | 0 | AA | 0 | 0.755179 |
| GG | 0.754696 | CC | 0 | CC | 0 | AA | 0 | 0.754696 |
| AA | 0 | CC | 0 | AC | 0.469438 | AG | 0.26806 | 0.737498 |
| AA | 0 | CT | 0.377831 | CC | 0 | AG | 0.26806 | 0.645892 |
| AG | 0.377348 | CC | 0 | CC | 0 | AG | 0.26806 | 0.645408 |
| AA | 0 | CC | 0 | CC | 0 | GG | 0.536121 | 0.536121 |
| AA | 0 | CC | 0 | AC | 0.469438 | AA | 0 | 0.469438 |
| AA | 0 | CT | 0.377831 | CC | 0 | AA | 0 | 0.377831 |
| AG | 0.377348 | CC | 0 | CC | 0 | AA | 0 | 0.377348 |
| AA | 0 | CC | 0 | CC | 0 | AG | 0.26806 | 0.26806 |
| AA | 0 | CC | 0 | CC | 0 | AA | 0 | 0 |

Table 13 (below) provides a proposed score of the varying genotype combinations, wherein the first two nucleotides of the combination refer to A1457G leptin SNP, the second two nucleotides refer to CRH, the third two nucleotides refer to TFAM 2 and the last two nucleotides refer to TFAM 3.

| | |
|---|---|
| PROPOSED SCORE | GENOTYPE COMBINATION |

| | |
|---|---|
| 10 | GGTTAAGG |
| 9 | GGTTAAAG |
| 9 | AGTTAAGG |
| 9 | GGCTAAGG |
| 9 | GGTTACGG |
| 8 | GGTTAAAA |
| 8 | AGTTAAAG |
| 8 | GGCTAAAG |
| 8 | GGTTACAG |
| 8 | AATTAAGG |
| 8 | AGCTAAGG |
| 8 | GGCCAAGG |
| 7 | AGTTACGG |
| 7 | GGCTACGG |
| 7 | AGTTAAAA |
| 7 | GGCTAAAA |
| 7 | GGTTCCGG |
| 7 | GGTTACAA |
| 7 | AATTAAAG |
| 7 | AGCTAAAG |
| 7 | GGCCAAAG |
| 7 | AGTTACAG |
| 7 | GGCTACAG |
| 7 | AACTAAGG |
| 7 | AGCCAAGG |
| 6 | GGTTCCAG |
| 6 | AATTACGG |
| 6 | AGCTACGG |
| 6 | GGCCACGG |
| 6 | AATTAAAA |
| 6 | AGCTAAAA |
| 6 | GGCCAAAA |
| 6 | AGTTCCGG |
| 6 | GGCTCCGG |
| 6 | AGTTACAA |
| 6 | GGCTACAA |
| 6 | AACTAAAG |
| 6 | AGCCAAAG |
| 6 | GGTTCCAA |
| 5 | AATTACAG |
| 6 | AGCTACAG |
| 5 | GGCCACAG |
| 5 | AACCAAGG |
| 5 | AGTTCCAG |
| 5 | GGCTCCAG |
| 5 | AACTACGG |
| 5 | AGCCACGG |
| 5 | AACTAAAA |
| 5 | AGCCAAAA |
| 5 | AATTCCGG |
| 5 | AGCTCCGG |
| 5 | GGCCCCGG |
| 5 | AATTACAA |
| 5 | AGCTACAA |
| 5 | GGCCACAA |
| 5 | AACCAAAG |
| 4 | AGTTCCAA |
| 4 | GGCTCCAA |
| 4 | AACTACAG |
| 4 | AGCCACAG |
| 4 | AATTCCAG |
| 4 | AGCTCCAG |
| 4 | GGCCCCAG |
| 4 | AACCACGG |
| 4 | AACCAAAA |
| 4 | AACTCCGG |
| 4 | AGCCCCGG |
| 4 | AACTACAA |
| 4 | AGCCACAA |
| 3 | AATTCCAA |
| 3 | AGCTCCAA |
| 3 | GGCCCCAA |
| 3 | AACCACAG |
| 3 | AACTCCAG |
| 3 | AGCCCCAG |
| 3 | AACCCCGG |
| 2 | AACCACAA |
| 2 | AACTCCAA |
| 2 | AGCCCCAA |
| 2 | AACCCCAG |
| 1 | AACCCCAA |

Having thus described in detail preferred embodiments of the present invention, it is to be understood that the invention defined by the above claims is not to be limited to particular details set forth in the above description as many apparent variations thereof are possible without departing from the spirit or scope of the present invention.

What is claimed is:

1. A method for identifying an animal having a desirable phenotype relating to dairy form or productive life, as compared to the general population of animals of that species, comprising determining the presence of a single nucleotide polymorphism in the leptin gene of the animal, wherein the polymorphism is selected from the group consisting of a cytosine (C) to thymine (T) substitution (C/T) at position 207 of the bovine leptin gene promoter, a cytosine (C) to thymine (T) substitution (C/T substitution) at position 528 of bovine leptin gene promoter, or a cytosine (C) to thymine (T) missense mutation at position 1759 in exon 2 of the coding region of the “wild type” bovine leptin gene, and the single nucleotide polymorphism is indicative of a desirable phenotype relating to dairy form or productive life.

2. A method for sub grouping animals according to genotype wherein the animals of each sub-group have a similar genotype in the leptin gene comprising:
(a) determining the genotype of each animal to be subgrouped in accordance with the method of claim 1 and
(b) segregating individual animals into sub-groups depending on whether the animals have, or do not have, a cytosine (C) to thymine (T) substitution (C/T) at position 207 of the bovine leptin gene promoter, a cytosine (C) to thymine (T) substitution (C/T substitution) at position 528 of bovine leptin gene promoter, or a cytosine (C) to thymine (T) missense mutation at position 1759 in exon 2 of the coding region of the “wild type” bovine leptin gene,
wherein the polymorphism in the leptin gene has an association with dairy form or productive life.

3. A method for identifying an animal having a desirable phenotype relating to productive life, as compared to the general population of animals of that species, comprising determining the presence of a single nucleotide polymorphism in the (a) leptin gene of the animal, wherein the polymorphism is an adenine (A) to guanine (G) substitution (A/G) at position 1540 of SEQ ID NO. 1, (b) the corticotropin-releasing hormone (CRH) gene of the animal, wherein the polymorphism is selected from the group consisting of AAFC03076794.1:g.9657C>T, c.10718G>C, c.10841G>A, c.10893A>C and c.10936G>C and (c) the mitochondrial transcription factor A (“TFAM”) gene of the animal, wherein the polymorphism is selected from the group consisting of an A to C substitution at the −1220 nucleotide position in the promoter of the TFAM gene, a T to C substitution at position −1212 in the promoter of the TFAM gene and a T to C substitution at position −995 in the promoter of the TFAM gene, and the single nucleotide polymorphism is indicative of a desirable phenotype relating to productive life.

4. A method for sub grouping animals according to genotype wherein the animals of each sub-group have a similar genotype in the leptin gene, CRH gene, TFAM gene or any combination thereof comprising:
(a) determining the genotype of each animal to be subgrouped in accordance with the method of claim 3 and
(b) segregating individual animals into sub-groups depending on whether the animals have, or do not have, a single nucleotide polymorphism in the (a) leptin gene of the animal, wherein the polymorphism is an adenine (A) to guanine (G) substitution (A/G) at position 1540 of SEQ ID NO. 1, (b) the CRH gene of the animal, wherein the polymorphism is selected from the group consisting of AAFC03076794.1:g.9657C>T, c. 10718G>C, c.10841G>A, c.10893A>C and c.10936G>C and (c) the TFAM gene of the animal, wherein the polymorphism is selected from the group consisting of an A to C substitution at the −1220 nucleotide position in the promoter of the TFAM gene, a T to C substitution at position −1212 in the promoter of the TFAM gene and a T to C substitution at position −995 in the promoter of the TFAM gene
wherein the polymorphism in the leptin gene, CRH gene, TFAM gene or any combination thereof, has an association with dairy form or productive life.

5. An interactive computer-assisted method for tracking the traits of dairy form and productive life of livestock bovines comprising, using a computer system comprising a programmed computer comprising a processor, a data storage system, an input device, an output device, and an interactive device, the steps of: (a) inputting into the programmed computer through the input device data comprising a breeding history of a bovine or herd of bovines, (b) inputting into the programmed computer through the input device data comprising a veterinary history of a bovine or herd of bovines, (c) correlating the veterinary data with the breeding history of the bovine or herd of bovines using the processor and the data storage system, and (d) outputting to the output device the breeding history and the veterinary history of the bovine or herd of bovines.

6. The method according to claim 5, wherein the computer system is an interactive system whereby modifications to the output of the computer-assisted method may be correlated according to the input from the interactive device.

7. The method according to claim 5, further comprising the steps of inputting into the programmed computer diagnostic data related to the health of the cow or herd of cows; and correlating the diagnostic data to the breeding and veterinary histories of the cow or herd of cows.

8. The method of claim 7 wherein the data comprises presence or absence of one or more of a single nucleotide polymorphism(s) of interest in the leptin gene.

9. The method of claim 8 wherein the single nucleotide polymorphism(s) of interest is selected from the group consisting of a cytosine (C) to thymine (T) substitution (C/T) at position 207 of the bovine leptin gene promoter, a cytosine (C) to thymine (T) substitution (C/T substitution) at position 528 of bovine leptin gene promoter, or a cytosine (C) to thymine (T) missense mutation at position 1759 in exon 2 of the coding region of the “wild type” bovine leptin gene.

10. The method of claim 7 wherein the data comprises presence or absence of one or more of a single nucleotide polymorphism(s) of interest in the leptin gene, CRH gene, TFAM gene or any combination thereof.

11. The method of claim 10 wherein the single nucleotide polymorphism(s) of interest is (a) an adenine (A) to guanine (G) substitution (A/G) at position 1540 of SEQ ID NO. 1, (b) selected from the group consisting of AAFC03076794.1:g.9657C>T, c. 10718G>C, c.10841G>A, c.10893A>C and c. 10936G>C of the CRH gene, (c) selected from the group consisting of an A to C substitution at the −1220 nucleotide position in the promoter of the TFAM gene, a T to C substitution at position −1212 in the promoter of the TFAM gene and a T to C substitution at position −995 in the promoter of the TFAM gene, or any combination thereof.
